SEQUENCE LISTING (18 sequences)

SEQ ID NO: 1 - protein, 127 aa, Artificial Sequence [sequence omitted]

SEQ ID NO: 2 - protein, 46 aa, Artificial Sequence
  Description of Artificial Sequence Synthetic polypeptide
SHYKQHCPPTPETSCATQIITFESFKENLKDFLLVIPFDCWEPVQE

SEQ ID NO: 3 - protein, 91 aa, Artificial Sequence
  Description of Artificial Sequence Synthetic polypeptide
EMNETVEVISEMFDLQEPTCQTRLELYKQGLRGSLTKLKGPLTMMSHYKQHCPPTPETSCATQIITFESFKENLKDFLLVIPFDCWEPVQE

SEQ ID NO: 4 - protein, 33 aa, Artificial Sequence
  Description of Artificial Sequence Synthetic polypeptide
APARSPSPSTQPWEHVNAIQEARRLLNLSRDTA

SEQ ID NO: 5 - protein, 126 aa, Artificial Sequence
  Description of Artificial Sequence Synthetic polypeptide
APARSPSPSTQPWEHVNAIQEARRLLNLSRDTACEMNETVEVISEMFDLQEPTCQTRLELYKQGLRGSLTKLKGPLTMMCSHYKQHCPPTPETSCATQIITFESFKENLKDFLLVIPFDCWEPVQE

SEQ ID NO: 6 - protein, 126 aa, Artificial Sequence
  Description of Artificial Sequence Synthetic polypeptide
APARSPSPSTQPWEHVNAIQEARRLLNLSRDTAAEMNETVEVISEMFDLQEPTCQTRLELYKQGLRGSLTKLKGPLTMMASHYKQHCPPTPETSCATQIITFESFKENLKDFLLVIPFDCWEPVQE

SEQ ID NO: 7 - protein, 40 aa, Artificial Sequence
  Description of Artificial Sequence Synthetic polypeptide
LQTRLELYKQGLRGSLTKLKGPLTMMASHYKQHCPPTPET

SEQ ID NO: 8 - protein, 33 aa, Artificial Sequence; identical to SEQ ID NO: 4
  Description of Artificial Sequence Synthetic polypeptide

SEQ ID NO: 9 - protein, 19 aa, Artificial Sequence
  Description of Artificial Sequence Synthetic peptide
EMNETVEVISEMFDLQEPT

SEQ ID NO: 10 - protein, 41 aa, Artificial Sequence
  Description of Artificial Sequence Synthetic polypeptide
LQTRLELYKQGLRGSLTKLKGPLTMMASHYKQHCPPTPETS

SEQ ID NO: 11 - protein, 53 aa, Artificial Sequence
  Description of Artificial Sequence Synthetic polypeptide
APARSPSPSTQPWEHVNAIQEARRLLNLSRDTACEMNETVEVISEMFDLQEPT

SEQ ID NO: 12 - protein, 32 aa, Artificial Sequence
  Description of Artificial Sequence Synthetic polypeptide
CATQIITFESFKENLKDFLLVIPFDCWEPVQE

SEQ ID NO: 13 - protein, 53 aa, Artificial Sequence
  Description of Artificial Sequence Synthetic polypeptide
APARSPSPSTQPWEHVNAIQEARRLLNLSRDTAAEMNETVEVISEMFDLQEPT

SEQ ID NO: 14 - protein, 73 aa, Artificial Sequence
  Description of Artificial Sequence Synthetic polypeptide
LQTRLELYKQGLRGSLTKLKGPLTMMASHYKQHCPPTPETSCATQIITFESFKENLKDFLLVIPFDCWEPVQE

SEQ ID NO: 15 - protein, 126 aa, Artificial Sequence; identical to SEQ ID NO: 6
  Description of Artificial Sequence Synthetic polypeptide

SEQ ID NO: 16 - protein, 45 aa, Artificial Sequence
  Description of Artificial Sequence Synthetic polypeptide
EMDETVEVISEMFDLQEPTCQTRLELYKQGLRGSLTKLKGPLTMM

SEQ ID NO: 17 - protein, 45 aa, Artificial Sequence
  Description of Artificial Sequence Synthetic polypeptide
EMNETVEVISEMFDLQEPTCQTRLELYKQGLRGSLTKLKGPLTMM

SEQ ID NO: 18 - protein, 126 aa, Artificial Sequence; identical to SEQ ID NO: 6
  Description of Artificial Sequence Synthetic polypeptide

(19) United States
(12) Patent Application Publication
(10) Pub. No.: US 2016/0215033 A1
(43) Pub. Date: Jul. 28, 2016

(54) Granulocyte Macrophage Colony-Stimulating Factor Compositions

(71) Applicant: SLOAN-KETTERING INSTITUTE FOR CANCER RESEARCH, New York, NY (US)

(72) Inventors: Samuel J. Danishefsky, Englewood, NJ (US); Qiang Zhang, New York, NY (US); Eric Johnston, New York, NY (US)

(73) Assignee: Sloan-Kettering Institute for Cancer Research, New York, NY (US)

(21) Appl. No.: 14/916,027

(22) PCT Filed: Sep. 3, 2014
(86) PCT No.: PCT/US2014/053895

§ 371 Date: Mar. 2, 2016

Related U.S. Application Data
(60) Provisional application No. 61/873,284, filed on Sep. 3, 2013.

(51) Int. Cl. C07K 14/535; A61K 39/39; A61K 39/00

(52) U.S. Cl. CPC C07K 14/535; A61K 39/0011; A61K 39/39; A61K 2039/55522; A61K 38/00

(57) ABSTRACT

The present invention provides a composition of homogeneously glycosylated GM-CSF or a homogeneously glycosylated fragment thereof, wherein each molecule of GM-CSF or fragment thereof has the same glycosylation pattern, and for a given glycosylation site each molecule of GM-CSF or fragment thereof has the same glycan. The present invention further provides methods of making and using such compositions.

### CROSS REFERENCE TO RELATED APPLICATIONS

The present invention claims priority to U.S. provisional patent application No. 61/873,284, filed Sep. 3, 2013, the entire contents of which are hereby incorporated by reference.

### GOVERNMENT SUPPORT

This invention was made with United States Government support under grants 7R01HL25848-33 and 9R01GM109760-34A1, awarded by the National Institutes of Health. The United States Government has certain rights in the invention.

### BACKGROUND

Over the past 30 years, cytokines have been increasingly used in the treatment of hematologic and oncologic diseases (Robak T., Arch Immunol Ther Exp (Warsz). 1996; 44(1):5-9; Charles A. Dinarello, Eur J Immunol. 2007 November; 37(Suppl 1): S34-S45). They are usually proteins or glycoproteins that are secreted by the human body. The main function of cytokines is to stimulate cellular growth and cell proliferation. Among many cytokines, granulocyte macrophage colony-stimulating factor (GM-CSF) has shown great biologic and therapeutic promise and consequently has become a target for numerous biological and clinical studies (FIG. 1) (Hamilton and Anderson Growth Factors, December 2004 Vol. 22 (4), pp. 225-231). GM-CSF is routinely secreted by the human immune system and acts as a signaling substrate, which stimulates stem cells to produce white blood cells in the bone marrow. In addition, GM-CSF controls the production, differentiation, and function of dendritic cells, as well as potentiates the responses of CD4+ T cells in vivo (Mellman I, Steinman R M. Dendritic cells: specialized and regulated antigen processing machines. Cell. 2001; 106:255-258; Barouch D H, Santra S, Tenner-Racz K, et al. Potent CD4+ T cell responses elicited by a bicistronic HIV1 DNA vaccine expressing gp120 and GM-CSF. J Immunol. 2002; 168:562-568). Due to these unique properties, GM-CSF has been used clinically to stimulate the production of white blood cells in patients undergoing chemotherapy and autologous bone marrow transplants to alleviate the compromising effects on their immune systems. More recently, it has also been evaluated in clinical trials for its potential as a vaccine adjuvant in HIV-infected patients (Borrello I, Pardoll D., Cytokine Growth Factor Rev. 2002 April; 13(2):185-93). Presently, GM-CSF is mainly obtained from recombinant technologies involving yeast and Chinese Hamster Ovary (CHO) cells. However, the drawback of this method is that the product GM-CSF is obtained as a complex mixture of glycoforms due to lack of transcript pattern. Interestingly, it can also be derived from E. coli, which generate an aglycone peptide backbone free of any carbohydrates. Evaluations of the difference between the glycopeptide and aglycone reveal that glycosylated GM-CSF not only benefits from having better pharmacokinetic properties, but it is also associated with less adverse reactions, such as bone pain and dyspnea (C. Denzlinger, W. Tetzloff, H. H. Gerhartz, R. Pokorny, S. Sagebiel, C. Haberl, and W. Wilmanns, Blood, Vol 81, No 8(Apr. 15). 1993: pp 2007-2013; Jacob M. Rowe, Clinical Infectious Diseases 1998; 26:1290-4). Homogeneous glycoproteins cannot be obtained by current recombinant technologies.

### SUMMARY OF THE INVENTION

The present invention provides, among other things, a composition of homogeneously glycosylated GM-CSF or a homogeneously glycosylated fragment thereof, wherein each molecule of GM-CSF or fragment thereof has the same glycosylation pattern, and for a given glycosylation site each molecule of GM-CSF or fragment thereof has the same glycan. The present invention also provides, among other things, a polypeptide whose amino acid sequence includes a sequence that contains one or more modifications relative to that of SEQ ID NO: 1, wherein at least one such modification prevents or decreases the polypeptide's susceptibility to truncation relative to that of a polypeptide whose sequence is identical to SEQ ID NO: 1. The present invention also provides, among other things, a prodrug of homogeneously glycosylated GM-CSF or a homogeneously glycosylated fragment thereof, wherein the GM-CSF polypeptide's C- or N-terminus is modified such that, upon suitable in vivo bioactivation, the prodrug is converted to an active form of GM-CSF. The present invention further provides methods of making and using provided compositions, including for example methods of stimulating white blood cell production and methods of enhancing the immune response to a cancer vaccine.

### BRIEF DESCRIPTION OF THE DRAWINGS

FIG. 1 depicts a three dimensional structure of GM-CSF.

FIG. 2 depicts a GM-CSF sequence.

FIG. 3 depicts a synthetic plan for GM-CSF aglycone.

FIG. 4 depicts a synthetic plan for glycosylated GM-CSF.

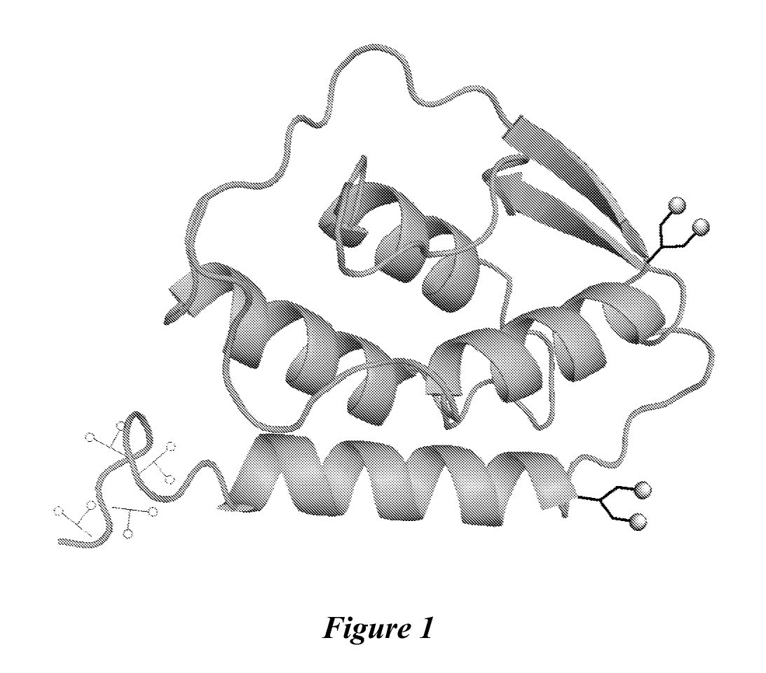

FIG. S1 depicts a LC-MS trace and ESI-MS analysis of peptide 2.

FIG. S2 depicts a LC-MS trace and ESI-MS analysis of peptide 3.

FIG. S3 depicts a LC-MS trace and ESI-MS analysis of peptide 6.

FIG. S4 depicts a LC-MS trace and ESI-MS analysis of peptide 5.

FIG. S5 depicts UV and MS traces from LC-MS analysis of peptide 4.

FIG. S6 depicts UV and MS traces from LC-MS analysis of peptide 8.

FIG. S7 depicts UV and MS traces from LC-MS analysis of peptide 9.

FIG. S8 depicts UV and MS traces from LC-MS analysis of peptide 15.

FIG. S9 depicts UV and MS traces from LC-MS analysis of peptide 14.

FIG. S10 depicts UV and MS traces from LC-MS analysis of peptide 17.

FIG. S11 depicts a LC-MS trace and ESI-MS analysis of glycopeptide 16.

FIG. S12 depicts a LC-MS trace and ESI-MS analysis of glycopeptide S6.

FIG. S13 depicts a LC-MS trace and ESI-MS analysis of glycopeptide S7.

FIG. S14 depicts a LC-MS trace and ESI-MS analysis of glycopeptide 22.

FIG. S15 depicts a LC-MS trace and ESI-MS analysis of glycopeptide 18.

FIG. S16 depicts a LC-MS trace and ESI-MS analysis of glycopeptide 19.

FIG. S17 depicts a LC-MS trace and ESI-MS analysis of glycopeptide 20.

FIG. S18 depicts a LC-MS trace and ESI-MS analysis of glycopeptide 10.

FIG. S19 depicts SDS-PAGE of glycopeptides 10, 21, 23. Gel cassette was load with synthetic compounds 10, 21 and 23 along with commerically available GM-CSFs, after an electric field is applied across the gel for 2 hours, gel was stained with Coomassie Blue. Lane a: recombinant aglycone GM-CSF; Lane b: synthetic GM-CSF aglycone 10, Lane c: synthetic glycosylated GM-CSF 23, Lane d: synthetic glycosylated GM-CSF 21, Lane e: recombinant glycosylated GM-CSF, Lane f: recombinant glycosylated GM-CSF with double concentration.

FIG. S20 depicts the effect of synthetic and recombinant GM-CSF on TF-1 cell proliferation.

FIG. S21 depicts the effect of synthetic and recombinant GM-CSF on colony formation in cord blood CD34+ cells.

FIG. S22 depicts images of colonies formed in CB CD34+ cells after 14 days of GM-CSF/KL stimulation. Each GM-CSF group has multiple images. Only one representative image of each group was shown.

FIG. S23 depicts CD spectra of synthetic GM-CSFs compared to recombinant GM-CSF aglycone: (a) synthetic GM-CSF aglycone 10; (b) recombinant GM-CSF aglycone; (c) bis-glycosylated GM-CSF 21.

### DETAILED DESCRIPTION OF CERTAIN EMBODIMENTS

There are a number of naturally occurring heterogenous glycoproteins that demonstrate clinical importance (Ping Wang, et al.; Angewandte Chemie International Edition Volume 51, Issue 46, pages 11576-11584, Nov. 12, 2012). The present disclosure describes the chemical total synthesis of GM-CSF in both glycosylated and non-glycosylated forms. Access to these constructs allows investigation of the biological activities of both forms of this protein. In addition, it will be appreciated that synthetic access allows for modification the protein sequence and its glycosylation pattern in order to study, for example, structure-activity relationships and the activity of new GM-CSF analogs.

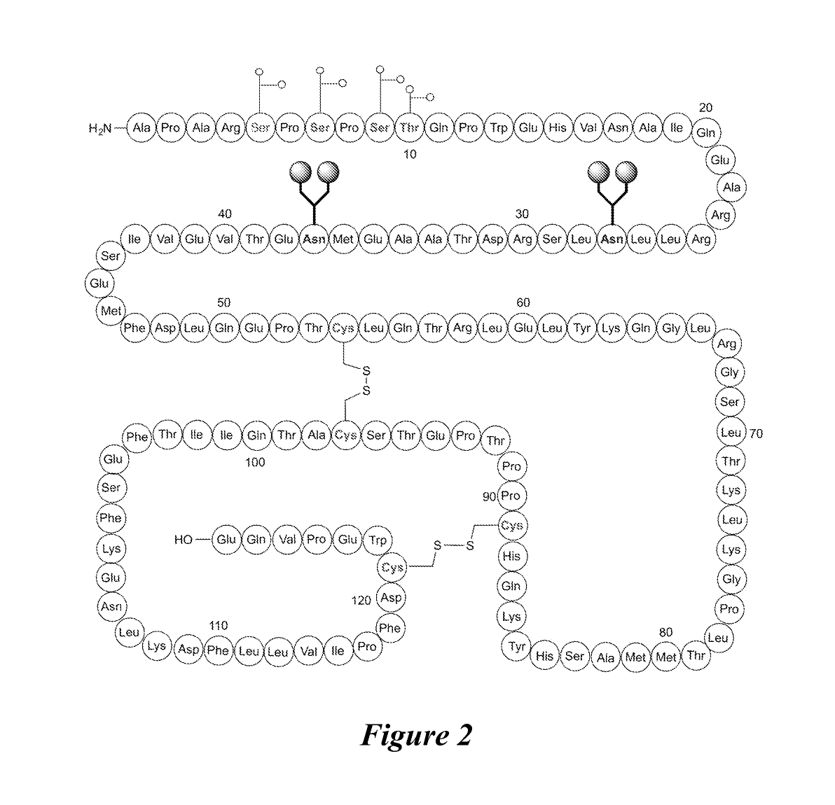

Structurally, GM-CSF consists of 127 amino acids with two N-linked oligosaccharides located at Asn²⁷ and Asn³⁷ (FIG. 2) (Kaushansky, K., Biochemistry 1992, 31,1881. Donahue, R. E., Cold Spring Harbor Symp. Quant. Biol. 1986, 51, 685). Interestingly, the location and the number of the O-linked carbohydrates are still controversial, ranging from two oligosaccharides at Ser⁷ and Ser⁹/Thr¹⁰ to four carbohydrates at Ser⁵, Ser⁷, Ser⁹ and Thr¹⁰ positions (Forno, G., Eur. J. Biochem. 2004, 271, 907). Two cross-linked disulfide bonds at Cys^{54,97} and CyS^{89,121} are responsible for the tertiary structure of GM-CSF by guiding the protein folding.

The discovery of native chemical ligation (NCL) by Kent and coworkers has profoundly changed the underlying strategy for performing protein synthesis (Dawson, P. E.; Muir, T. W.; Clark-Lewis, I.; Kent, S. B., Science, 1994, 266, 776-778), and the introduction of a metal-free desulfurization procedure has further expanded the scope of the NCL method (Wan, Q.; Danishefsky, S. J. Angew Chem Int Ed Engl 2007, 46, 9248-9252). Such procedures, along with those known in the art and others described herein, are used in the ensuing Examples to provide, for example, GM-CSF aglycone and homogeneous glycoforms. Analytical and biological studies confirm the structure and activity of these synthetic congeners.

Glycosylation has been reported to increase the survival of GM-CSF as well as to confer direct resistance to proteolysis which, in turn, is believed to be responsible for a longer half-life (Cebon J, Nicola N, Ward M, et al. Granulocyte-macrophage colony-stimulating factor from human lymphocytes. The effect of glycosylation on receptor binding and biologic activity. J Biol Chem 1990; 265:4483-91). Glycosylation is also believed to be important in the augmentation of binding to plasma proteins for transport (Ashwell G, Morell A G. The role of surface carbohydrates in the hepatic recognition and transport of circulating glycoproteins. Adv Enzymol Relat Areas Mol Biol 1974; 41:99-128). In addition, there has been much speculation regarding the dependence of survival and stimulation of monocytes on the carbohydrate moiety and its influence on in vivo activity (Moonen P, Mermod J J, Ernst J F, et al. Increased biological activity of deglycosylated recombinant human granulocyte/macrophage colony-stimulating factor produced by yeast or animal cells. Proc Natl Acad Sci USA 1987; 84:4428-31).

Purifying GM-CSF from living organisms or cells leads to heterogeneous mixtures of various glycosylated forms of GM-CSF; therefore, to date, a homogeneous composition of glycosylated GM-CSF has not been achieved. The present invention encompasses the recognition that compositions of homogeneously glycosylated GM-CSF, wherein all the molecules of the composition have the same, identical glycosylation pattern, can provide therapeutics having increased potency, stability, and/or safety.

In some embodiments, the present invention provides homogeneously glycosylated GM-CSF. In some embodiments, the present invention provides homogeneously glycosylated full-length GM-CSF. In some embodiments, the present invention provides homogeneous, fully-glycosylated full-length GM-CSF.

In some embodiments, the present invention provides homogeneous, fully glycosylated GM-CSF. In some embodiments, the present invention provides homogeneous, fully glycosylated GM-CSF glycosylated at Asn²⁷, Asn³⁷, Ser⁵, Ser⁷, Ser⁹, and Thr¹⁰.

In some embodiments, the present invention provides a composition of homogeneously glycosylated GM-CSF or a homogeneously glycosylated fragment thereof, wherein each molecule of GM-CSF or fragment thereof has the same glycosylation pattern, and for a given glycosylation site each molecule of GM-CSF or fragment thereof has the same glycan.

In some embodiments, a composition comprises a polypeptide whose amino acid sequence includes a sequence that:
a) is identical to that of:

(SEQ ID NO: 1)
Ala-Pro-Ala-Arg-Ser-Pro-Ser-Pro-Ser-Thr-Gln-Pro-
Trp-Glu-His-Val-Asn-Ala-Ile-Gln-Glu-Ala-Arg-Arg-
Leu-Leu-Asn-Leu-Ser-Arg-Asp-Thr-Ala-Ala-Glu-Met-
Asn-Glu-Thr-Val-Glu-Val-Ile-Ser-Glu-Met-Phe-Asp-
Leu-Gln-Glu-Pro-Thr-Cys-Leu-Gln-Thr-Arg-Leu-Glu-
Leu-Tyr-Lys-Gln-Gly-Leu-Arg-Gly-Ser-Leu-Thr-Lys-
Leu-Lys-Gly-Pro-Leu-Thr-Met-Met-Ala-Ser-His-Tyr-
Lys-Gln-His-Cys-Pro-Pro-Thr-Pro-Glu-Thr-Ser-Cys-
Ala-Thr-Gln-Ile-Ile-Thr-Phe-Glu-Ser-Phe-Lys-Glu-
Asn-Leu-Lys-Asp-Phe-Leu-Leu-Val-Ile-Pro-Phe-Asp-
Cys-Trp-Glu-Pro-Val-Gln-Glu,

or
b) contains 1, 2, 3, 4, 5, 6, 7, 8, 9, or 10 amino acid deletions, substitutions, additions or combinations thereof relative to such SEQ ID NO: 1, or
c) is a fragment of a) or b), wherein the fragment has an amino acid sequence corresponding to amino acid residues 1-33, 34-53, 34-80, 54-95, 81-127, or 96-127 of SEQ ID NO: 1, or contains 1, 2, 3, 4, 5, 6, 7, 8, 9, or 10 amino acid deletions, substitutions, additions or combinations thereof relative to such fragment;
   the polypeptide having at least one amino acid residue site glycosylated;
   wherein each glycosylated polypeptide in the composition has the same glycosylation pattern in that:
   it is glycosylated on at least one amino acid residue site;
it is glycosylated at the same at least one site;
it is glycosylated at a site selected from the group consisting of Asn²⁷, Asn³⁷, Ser⁵, Ser⁷, Ser⁹, and Thr¹⁰, in SEQ ID NO:1, and combinations thereof; and
   for a given glycosylation site, it has the same glycan.

In some embodiments of provided compositions, a polypeptide's amino acid sequence is identical to that of SEQ ID NO: 1. In certain embodiments, a polypeptide's amino acid sequence is SEQ ID NO: 1 having 1, 2, 3, 4, 5, 6, 7, 8, 9, or 10 amino acid deletions, substitutions, additions or combinations thereof.

In some embodiments of provided compositions, a polypeptide's amino acid sequence includes a sequence that contains one or more modifications relative to that of SEQ ID NO: 1, wherein at least one such modification prevents or decreases the polypeptide's susceptibility to truncation relative to that of a polypeptide whose sequence is identical to SEQ ID NO: 1. In some embodiments, a modification is the addition of or substitution with one, two, three, four, five, six, seven, or more unnatural amino acids. In certain embodiments, a modification is glycosylation of one or more amino acid residues. In some embodiments, a modification prevents or decreases the polypeptide's susceptibility to truncation at the N-terminus relative to that of a polypeptide whose sequence is identical to SEQ ID NO: 1. In some embodiments, a modification prevents or decreases the polypeptide's susceptibility to truncation by dipeptidyl peptidase 4 relative to that of a polypeptide whose sequence is identical to SEQ ID NO: 1.

In some embodiments of provided compositions, a provided GM-CSF fragment has an amino acid sequence corresponding to amino acid residues 1-33, 34-53, 34-80, 54-95, 81-127, or 96-127 of SEQ ID NO: 1, or contains 1, 2, 3, 4, 5, 6, 7, 8, 9, or 10 amino acid deletions, substitutions, additions or combinations thereof relative to such fragment.

In some embodiments of provided compositions, the structure of a provided GM-CSF fragment is selected from:

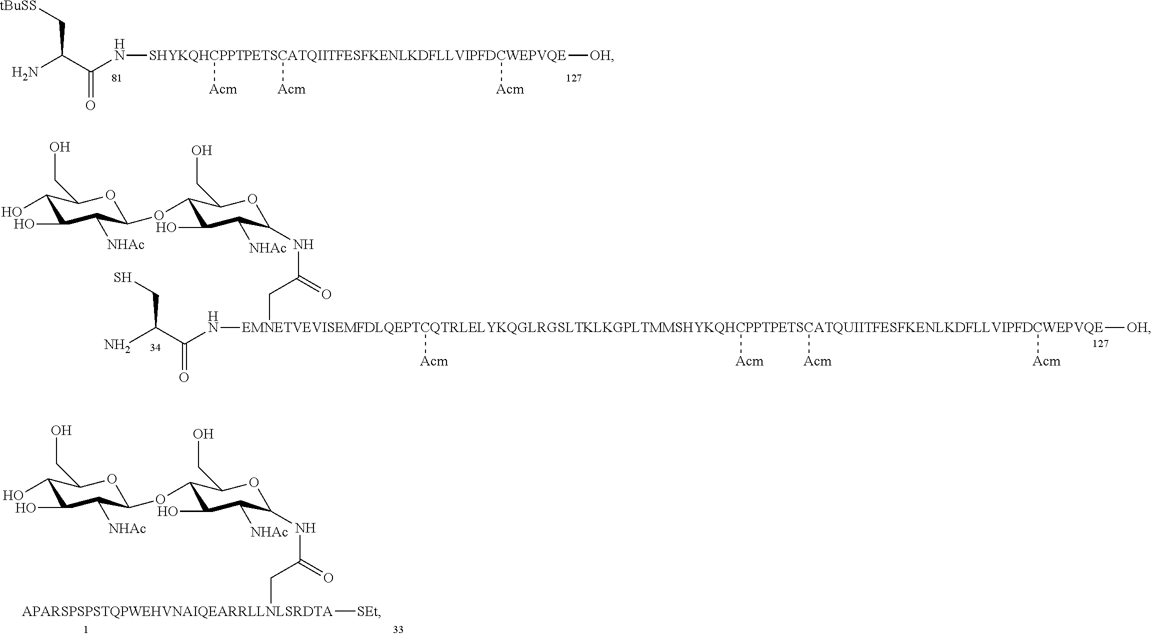

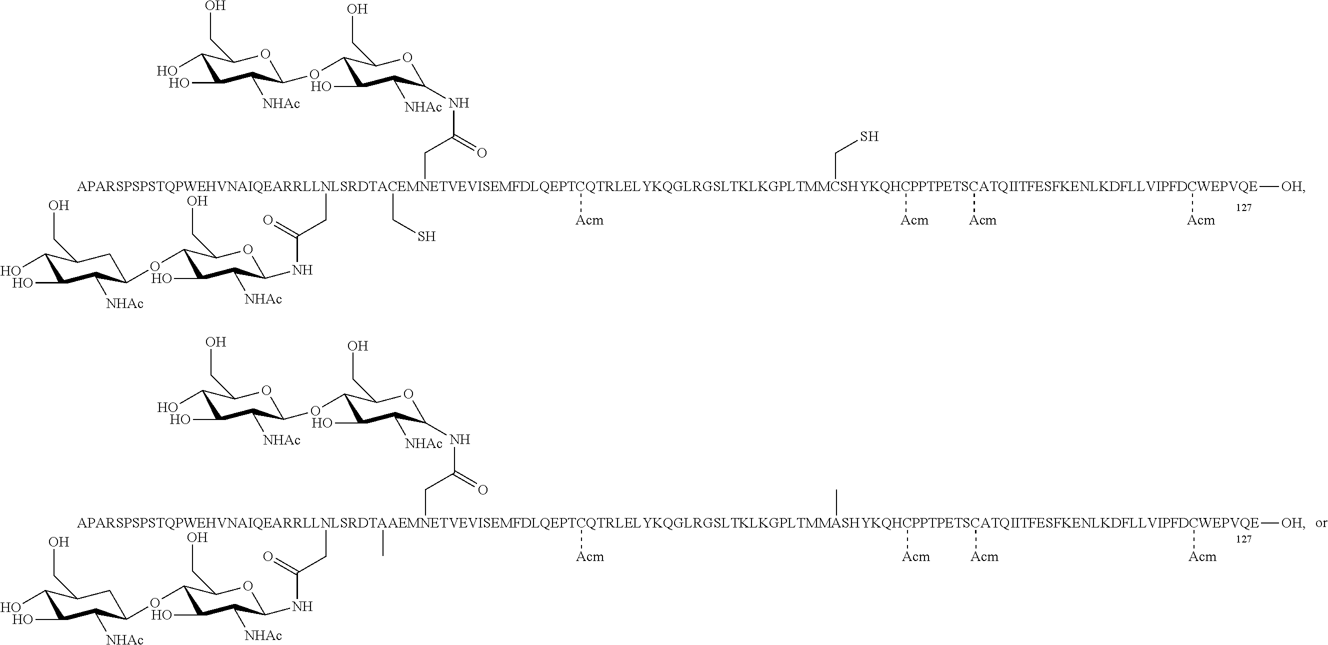

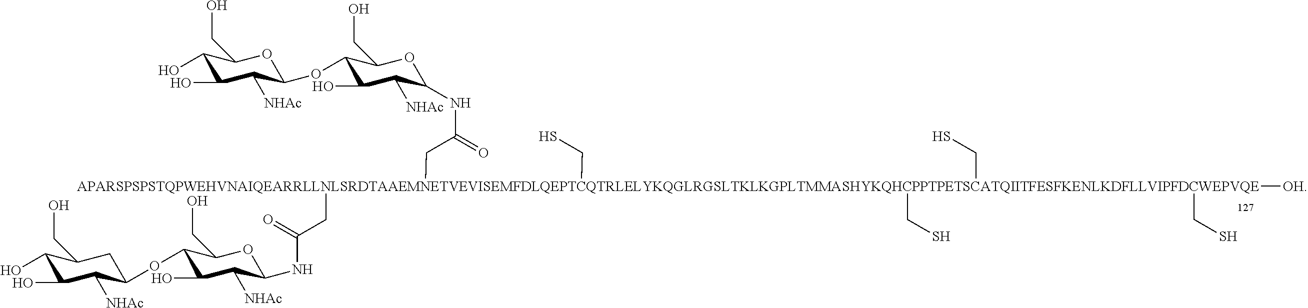

In some embodiments, the present invention provides a polypeptide whose amino acid sequence includes a sequence that contains one or more modifications relative to that of SEQ ID NO: 1, wherein at least one such modification prevents or decreases the polypeptide's susceptibility to truncation relative to that of a polypeptide whose sequence is identical to SEQ ID NO: 1. In some embodiments, a modification is the addition of or substitution with one, two, three, four, five, six, seven, or more unnatural amino acids. As used herein, the phrase “unnatural amino acid” refers amino acids not included in the list of 20 amino acids naturally occurring in proteins, as understood in the art. Such amino acids include the D-isomer of any of the 20 naturally occurring amino acids. Unnatural amino acids also include homoserine, ornithine, norleucine, and thyroxine. Other unnatural amino acids side-chains are well known to one of ordinary skill in the art and include unnatural aliphatic side chains. Other unnatural amino acids include modified amino acids, including those that are N-alkylated, cyclized, phosphorylated, acetylated, amidated, azidylated, labelled, and the like. In some embodiments, an unnatural amino acid is a D-isomer. In some embodiments, an unnatural amino acid is a L-isomer.

In some embodiments, the modification is glycosylation of one or more amino acid residues. In certain embodiments, a modification prevents or decreases the polypeptide's susceptibility to truncation at the N-terminus relative to that of a polypeptide whose sequence is identical to SEQ ID NO: 1. In certain embodiments, a modification prevents or decreases the polypeptide's susceptibility to truncation by dipeptidyl peptidase 4 relative to that of a polypeptide whose sequence is identical to SEQ ID NO: 1. In some embodiments, a modification is the replacement of one or more L-amino acids of SED ID NO:1 with its D-amino acid counterpart. In some embodiments, a provided polypeptide further comprises 1, 2, 3, 4, 5, 6, 7, 8, 9, or 10 amino acid deletions, substitutions, additions or combinations thereof relative to such SEQ ID NO: 1.

In some embodiments, the homogeneous GM-CSF has mutations in its primary amino acid sequence. In some embodiments, the homogeneous GM-CSF has mutations in its primary amino acid sequence wherein Asn²⁷, Asn³⁷, Ser⁵, Ser⁷, Ser⁹, and Thr¹⁰ are not mutated. In some embodiments, the homogeneous GM-CSF has 1-20 amino acid substitutions, additions, and/or deletions. In some embodiments, the homogeneous GM-CSF has 1-20 amino acid substitutions, additions, and/or deletions wherein Asn²⁷, Asn³⁷, Ser⁵, Ser⁷, Ser⁹, and Thr¹⁰ are not mutated. In some embodiments, the homogeneous GM-CSF has 1-15 amino acid substitutions, additions, and/or deletions. In some embodiments, the homogeneous GM-CSF has 1-15 amino acid substitutions, additions, and/or deletions wherein Asn²⁷, Asn³⁷, Ser⁵, Ser⁷, Ser⁹, and Thr¹⁰ are not mutated. In some embodiments, the homogeneous GM-CSF has 1-10 amino acid substitutions, additions, and/or deletions. In some embodiments, the homogeneous GM-CSF has 1-10 amino acid substitutions, additions, and/or deletions wherein Asn²⁷, Asn³⁷, Ser⁵, Ser⁷, Ser⁹, and Thr¹⁰ are not mutated. In some embodiments, the homogeneous GM-CSF has 1-5 amino acid substitutions, additions, and/or deletions. In some embodiments, the homogeneous GM-CSF has 1-5 amino acid substitutions, additions, and/or deletions wherein Asn²⁷, Asn³⁷, Ser⁵, Ser⁷, Ser⁹, and Thr¹⁰ are not mutated. In some embodiments, provided GM-CSF mutants or variants are characterized in that they have at least 50%, at least 60%, at least 70%, at least 80%, at least 90%, at least 95%, at least 100%, or greater than 100% of the activity of homogenous or non-homogeneous (i.e., recombinant) fully-glycosylated GM-CSF.

In some embodiments, a homogenously glycosylated GM-CSF comprises a glycosylation site other than Asn²⁷, Asn³⁷, Ser⁵, Ser⁷, Ser⁹, and Thr¹⁰ in SEQ ID NO: 1. In some embodiments, the present application provides methods for the synthesis of homogenously glycosylated GM-CSF comprising glycosylation sites other than Asn²⁴, Asn³⁸, Asn⁸³, and Ser¹²⁶ in SEQ ID NO: 1, for example, by introducing glycosylation at a given site of a peptide fragment before ligation. Synthetic methods for introducing a glycosylated amino acid residue into a peptide fragment is extensively described herein and widely known in the art, including but not limited to those described in International Application Publication Number WO2007/120614, the entirety of which is hereby incorporated by reference.

In some embodiments, the primary sequence of a homogenously glycosylated GM-CSF is SEQ ID NO: 1. In some embodiments, the primary sequence of a homogenously glycosylated GM-CSF is SEQ ID NO: 1 contains 1, 2, 3, 4, 5, 6, 7, 8, 9, or 10 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, 3, 4, 5, 6, 7, 8, or 9 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, 3, 4, 5, 6, 7, or 8 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, 3, 4, 5, 6, or 7 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, 3, 4, 5, or 6 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, 3, 4, or 5 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, 3, or 4 amino acid deletions, substitutions, additions or combinations thereof relative to such SEQ ID NO: 1. In some embodiments, the primary sequence of a homogenously glycosylated GM-CSF is SEQ ID NO: 1 contains 1, 2, or 3 amino acid deletions, substitutions, additions or combinations thereof relative to such SEQ ID NO: 1. In some embodiments, the primary sequence of a homogenously glycosylated GM-CSF is SEQ ID NO: 1 contains 1, or 2 amino acid deletions, substitutions, additions or combinations thereof relative to such SEQ ID NO: 1. In some embodiments, the primary sequence of a homogenously glycosylated GM-CSF is SEQ ID NO: 1 contains 1 amino acid deletions, substitutions, additions or combinations thereof relative to such SEQ ID NO: 1. An amino acid deletion, substitution, addition, or a combination thereof is introduced by deleting, substituting, or adding one or more amino acid residues during chemical synthesis of a peptide fragment. As understood by a person having ordinary skill in the art, among other things, the present invention also provides methods for introducing glycosylation at a substituted or added amino acid residue. In some embodiments, glycosylation at a substituted or added amino acid residue is introduced in the same way as that at a natural glycosylation site.

In some embodiments, a glycosylated fragment of GM-CSF contains 1-20 amino acid deletions, substitutions, additions or combinations thereof. In some embodiments, a glycosylated fragment of GM-CSF contains 1-18 amino acid deletions, substitutions, additions or combinations thereof. In some embodiments, a glycosylated fragment of GM-CSF contains 1-16 amino acid deletions, substitutions, additions or combinations thereof. In some embodiments, a glycosylated fragment of GM-CSF contains 1-15 amino acid deletions, substitutions, additions or combinations thereof. In some embodiments, a glycosylated fragment of GM-CSF contains 1-14 amino acid deletions, substitutions, additions or combinations thereof. In some embodiments, a glycosylated fragment of GM-CSF contains 1-12 amino acid deletions, substitutions, additions or combinations thereof. In some embodiments, a glycosylated fragment of GM-CSF contains 1-10 amino acid deletions, substitutions, additions or combinations thereof. In some embodiments, a glycosylated fragment of GM-CSF contains 1-8 amino acid deletions, substitutions, additions or combinations thereof. In some embodiments, a glycosylated fragment of GM-CSF contains 1-6 amino acid deletions, substitutions, additions or combinations thereof. In some embodiments, a glycosylated fragment of GM-CSF contains 1-4 amino acid deletions, substitutions, additions or combinations thereof. In some embodiments, a glycosylated fragment of GM-CSF contains 20 amino acid deletions, substitutions, additions or combinations thereof. In some embodiments, a glycosylated fragment of GM-CSF contains 18 amino acid deletions, substitutions, additions or combinations thereof. In some embodiments, a glycosylated fragment of GM-CSF contains 16 amino acid deletions, substitutions, additions or combinations thereof. In some embodiments, a glycosylated fragment of GM-CSF contains 15 amino acid deletions, substitutions, additions or combinations thereof. In some embodiments, a glycosylated fragment of GM-CSF contains 14 amino acid deletions, substitutions, additions or combinations thereof. In some embodiments, a glycosylated fragment of GM-CSF contains 12 amino acid deletions, substitutions, additions or combinations thereof. In some embodiments, a glycosylated fragment of GM-CSF contains 10 amino acid deletions, substitutions, additions or combinations thereof. In some embodiments, a glycosylated fragment of GM-CSF contains 9 amino acid deletions, substitutions, additions or combinations thereof. In some embodiments, a glycosylated fragment of GM-CSF contains 8 amino acid deletions, substitutions, additions or combinations thereof. In some embodiments, a glycosylated fragment of GM-CSF contains 7 amino acid deletions, substitutions, additions or combinations thereof. In some embodiments, a glycosylated fragment of GM-CSF contains 6 amino acid deletions, substitutions, additions or combinations thereof. In some embodiments, a glycosylated fragment of GM-CSF contains 5 amino acid deletions, substitutions, additions or combinations thereof. In some embodiments, a glycosylated fragment of GM-CSF contains 4 amino acid deletions, substitutions, additions or combinations thereof. In some embodiments, a glycosylated fragment of GM-CSF contains 3 amino acid deletions, substitutions, additions or combinations thereof. In some embodiments, a glycosylated fragment of GM-CSF contains 2 amino acid deletions, substitutions, additions or combinations thereof. In some embodiments, a glycosylated fragment of GM-CSF contains one amino acid deletion, substitution, or addition.

In some embodiments, a homogeneous GM-CSF polypeptide or composition thereof is folded. In some embodiments, the homogeneous GM-CSF is folded as found in nature. In some embodiments, the homogeneous GM-CSF forms secondary structure. In some embodiments, the homogeneous GM-CSF forms secondary structure as found in nature. In some embodiments, the homogeneous GM-CSF forms tertiary structure. In some embodiments, the homogeneous GM-CSF forms tertiary structure as found in nature. The secondary and tertiary structures can be characterized by chemical, biochemical and structural biology means including, but not limited to chromatography, mass spectrometry, X-ray crystallography, NMR spectroscopy, and dual polarization interferometry.

In certain embodiments, the present invention provides a prodrug of homogeneously glycosylated GM-CSF or a homogeneously glycosylated fragment thereof, wherein a GM-CSF polypeptide's C- or N-terminus is modified such that, upon suitable in vivo bioactivation, the prodrug is converted to an active form of GM-CSF. In some embodiments, a GM-CSF polypeptide's C- or N-terminus is extended by a peptide or modified peptide sequence that is cleaved upon suitable in vivo bioactivation to yield an active form of GM-CSF.

### Uses

In some embodiments, the provided polypeptides, compositions, and prodrugs thereof are useful in medicine. As described above, GM-CSF is known to stimulate stem cells to produce white blood cells in the bone marrow. Thus, in certain embodiments, provided polypeptides, compositions, and prodrugs thereof are useful to stimulate white blood cell production. In some embodiments, the present invention provides a method of stimulating white blood cell production comprising administering to a patient in need thereof a composition, polypeptide, or prodrug as described herein. In some embodiments, a patient is infected with HIV. In some embodiments, a patient is being treated or has been treated with chemotherapy. In some embodiments, a patient has undergone autologous bone marrow transplant. In certain embodiments, a patient is immune compromised.

As described above, GM-CSF controls among other things the production, differentiation, and function of dendritic cells, which are part of the immune machinery involved in responding to cancer vaccine therapies. Therefore, in some embodiments, the present invention provides a method of enhancing the immune response to a cancer vaccine comprising administering to a patient in need thereof a composition, polypeptide, or a prodrug as described herein. In some embodiments, a patient has been diagnosed with cancer. In some embodiments, a method further comprises co-administration with a cancer vaccine. In some embodiments, a cancer vaccine comprises one or more carbohydrates. In some embodiments, a cancer vaccine comprises a glycopeptide as described in U.S. Pat. Nos. 6,660,714, 7,160,856, 7,550,146, 7,879,335, 8,623,378, 7,854,934, 7,824,687, or 7,645,454, or International Patent Publication Nos. WO2011/156774 or WO2010/006343, the entirety of each of which is hereby incorporated by reference.

### Exemplary Synthesis of GM-CSF

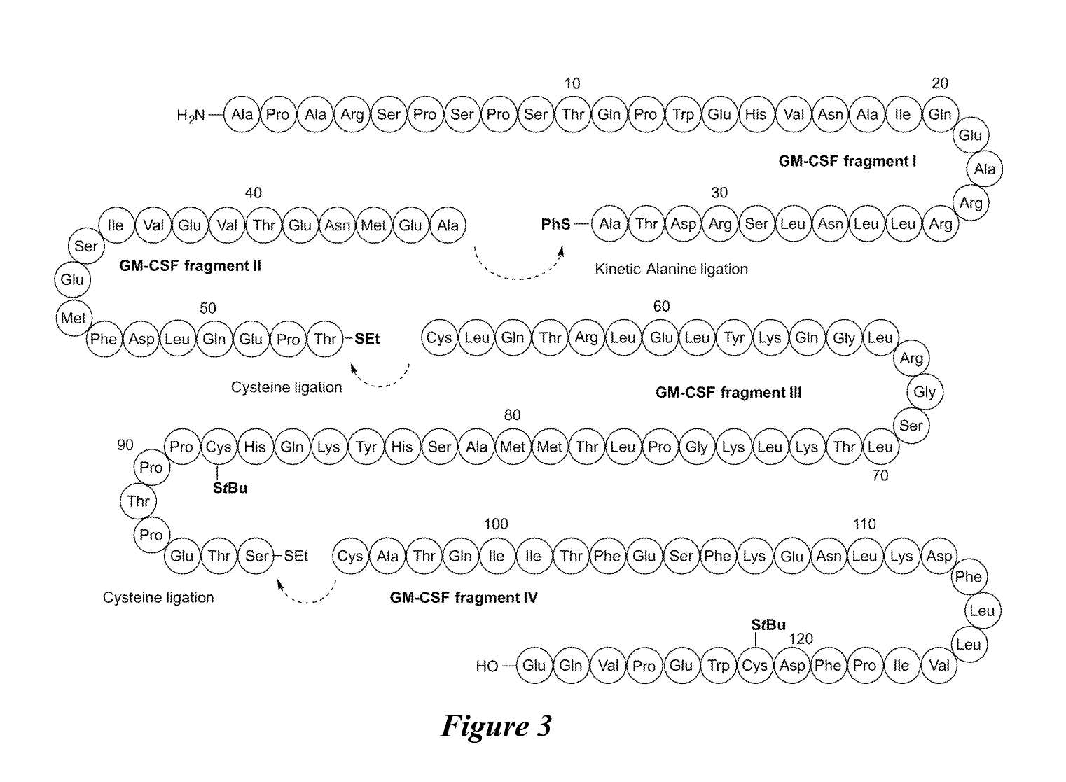

In some embodiments, the present disclosure dissects non-glycosylated GM-CSF into four fragments (FIG. 3), where the key connections would be the aniline ligation at Ala³³-Ala and cysteine ligations at Thr⁵³-Cys⁵⁴ and Ser⁹⁵-Cys⁹⁶. One advantage of this strategy would enable utilization of the maximum amount of cysteines (2 out 4) as ligation sites, thereby avoiding the late-stage removal of cysteine protecting groups. In order to achieve this goal, kinetic alanine ligation between Fragments I and II were used. In some embodiments, prior to any further ligation, a desulfurization at ligation site Cys (Ala)³⁴ is performed in the presence of a thioester at the C-terminal Thr⁵³ residue. Fragments III and IV may then be joined by an NCL with the rest of cysteine residues protected with t-butyl thioether, which would be liberated during the NCL reaction.

In some embodiments, GM-CSF synthesis commences with the preparation of Fragment IV (Scheme 1). While preparing the fully protected peptide sequence through Fmoc-based SPPS, unexpected aspartimide formation was observed (>90%). Further investigation revealed that aspartimide was formed at the Cys¹²⁰_Asp¹²¹ site. Replacing the Fmoc deblock reagent DBU with oxyma pure (Subiros-Funosas, R.; El-Faham, A.; Albericio, F. Biopolymers, 2012, 98, 89) successfully suppressed the formation of aspartimide and provided desired Fragment IV (1) in reasonable yield after “Cocktail B” (Huang H, Rabenstein D L., J Pept Res. 1999, 53(5):548-53) global deprotection. Polypeptide Thr⁵⁴-Pro⁹⁴ (2) was prepared by SPPS, and after cleavage from the resin by treatment with HOAc/TFE/CH₂Cl₂, it was subsequently coupled with the Ser⁹⁶-Set residue under known EDC coupling conditions to give the fully protected Fragment III. Finally, global deprotection of Fragment III led to the target peptide 3. Fragments I (5) and II (6) were also obtained in a similar manner in good yields.

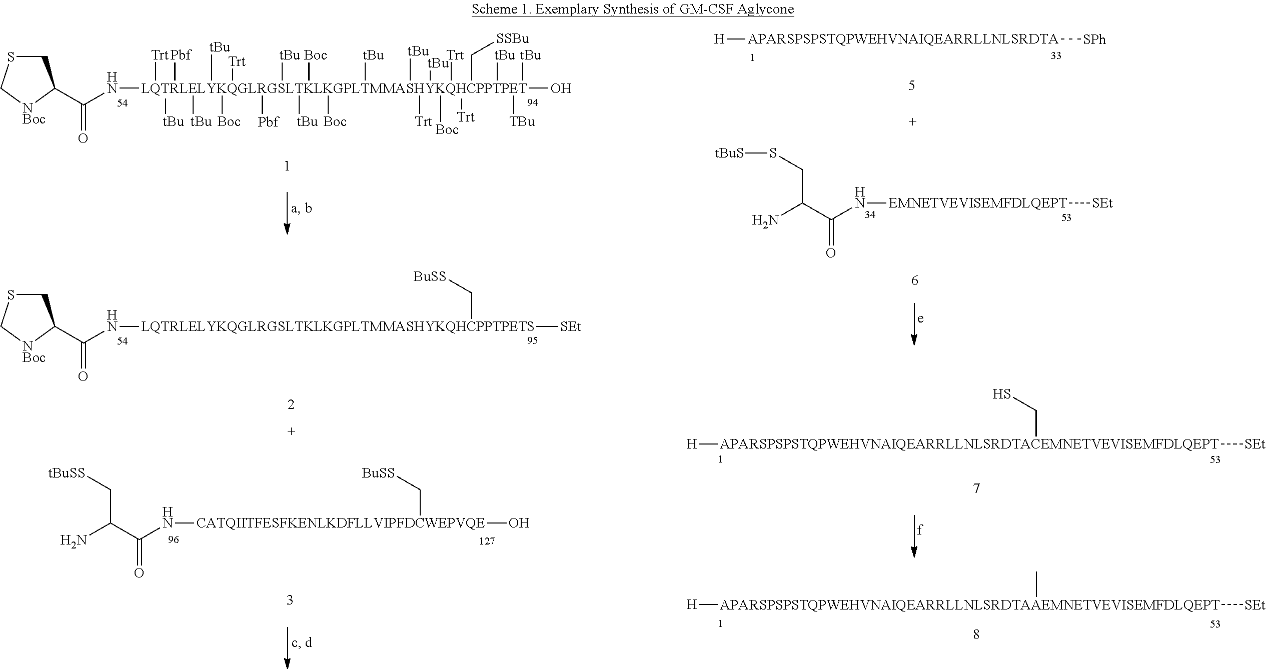

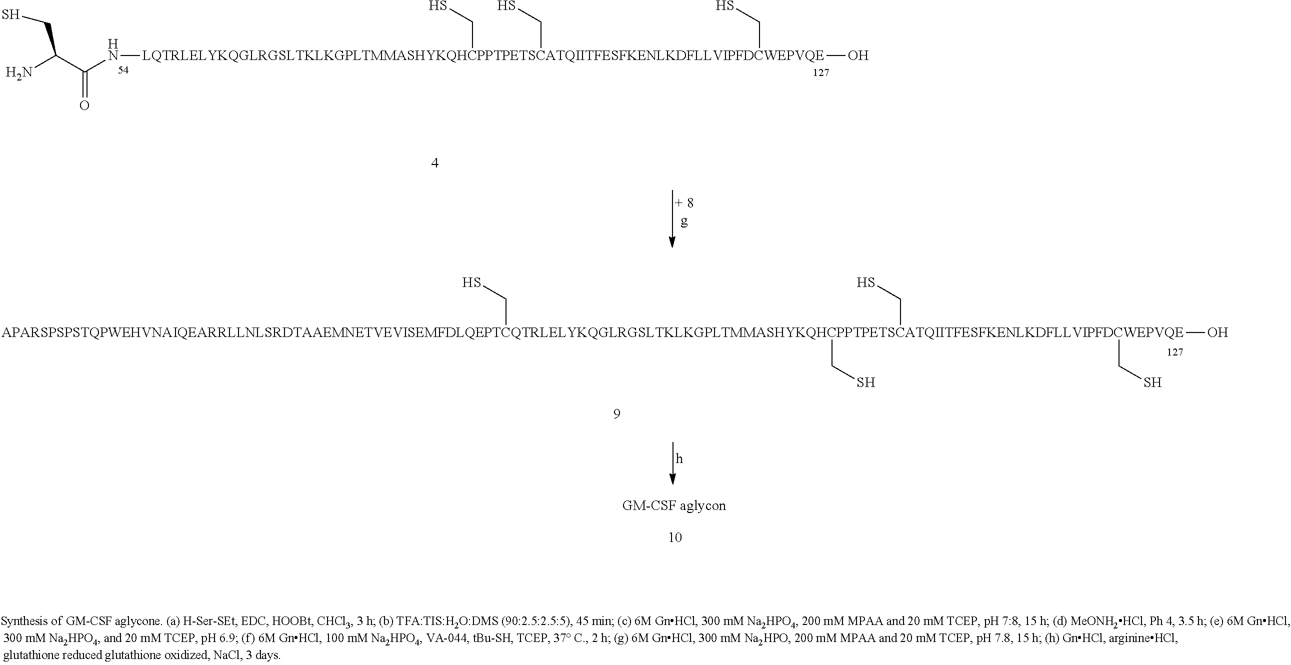

Treatment of peptides I (5) and II (6) in pH 7.0 kinetic NCL buffer (Bang D, Pentelute B L, Kent S B (2006) Angew Chem Int Ed Engl 45:3985-3988) afforded 7 with full conversion after 16 hours. The crude reaction mixture was then directly subjected to the desulfurization conditions, and to our delight, Cys³⁴ of 7 was smoothly reduced to Ala³⁴ in 8 with the Thr⁵⁴-Set functional group intact. Connection of Fragments III and IV followed by treatment with MeONH₂.HCl to provide construct 9 with all the cysteine residues deprotected. Convergent NCL coupling of polypeptides 8 and 9 afforded the primary sequence of non-glycosylated GM-CSF 10. Renaturation of construct 10 successfully formed folded GM-CSF aglycone 11 (Thomson C A, Olson M, Jackson L M, Schrader J W (2012). PLoS ONE 7(11): e49891).

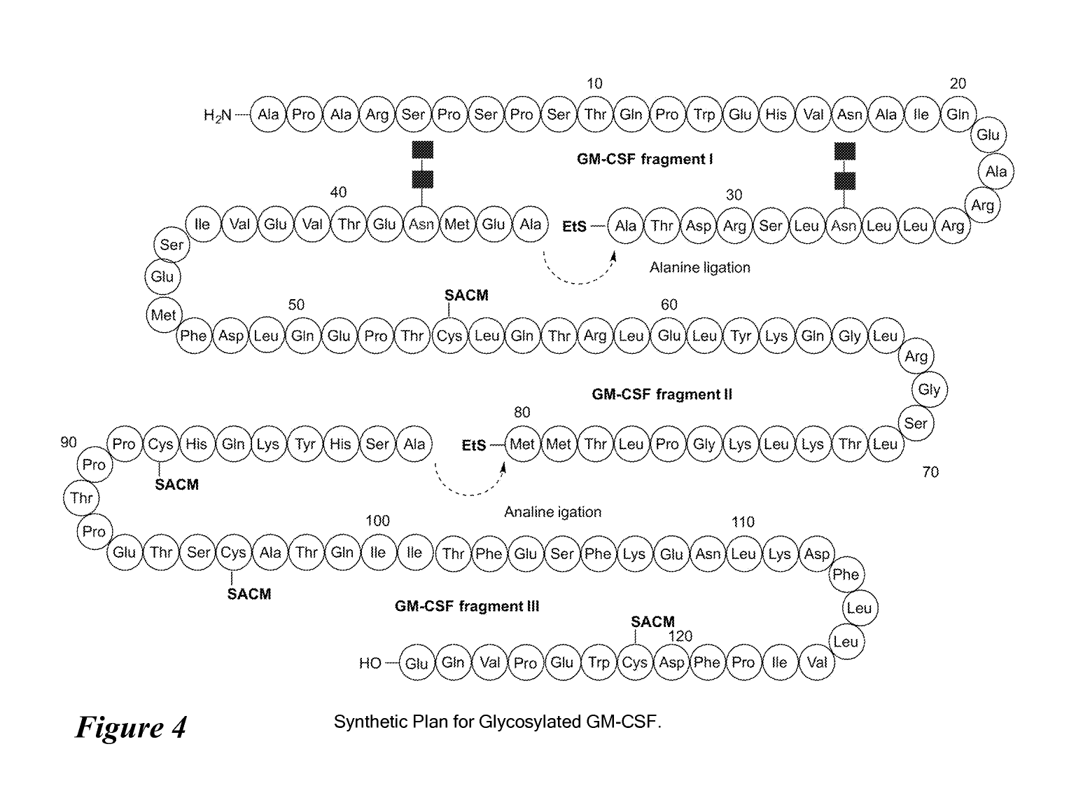

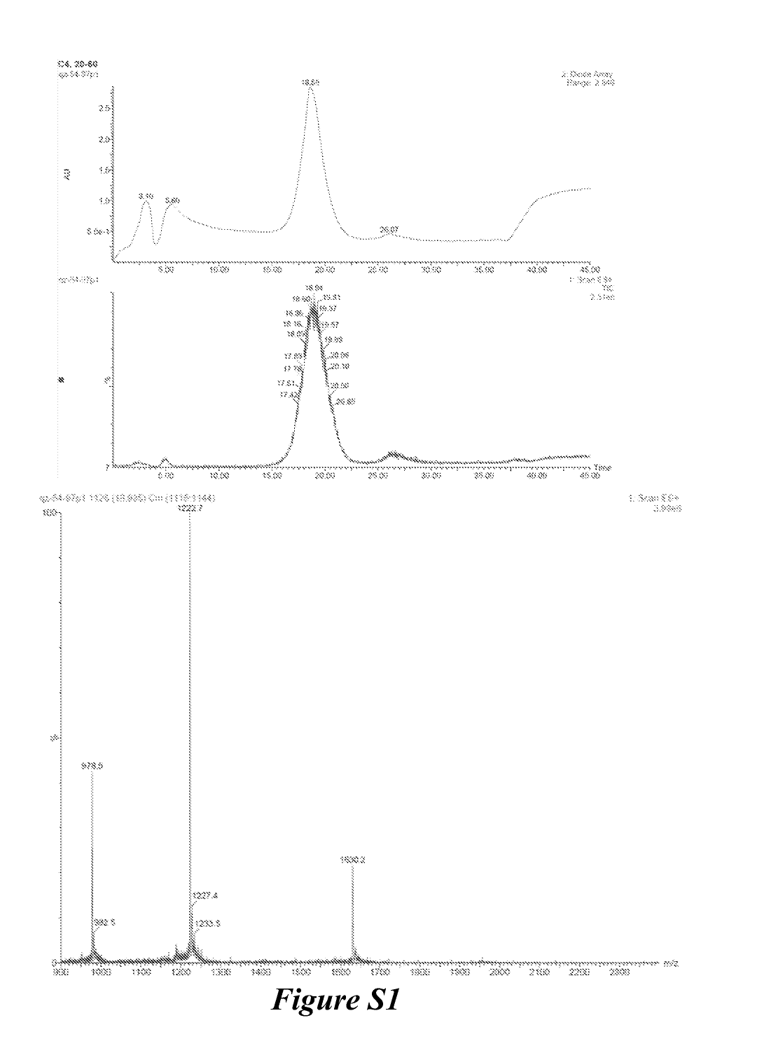

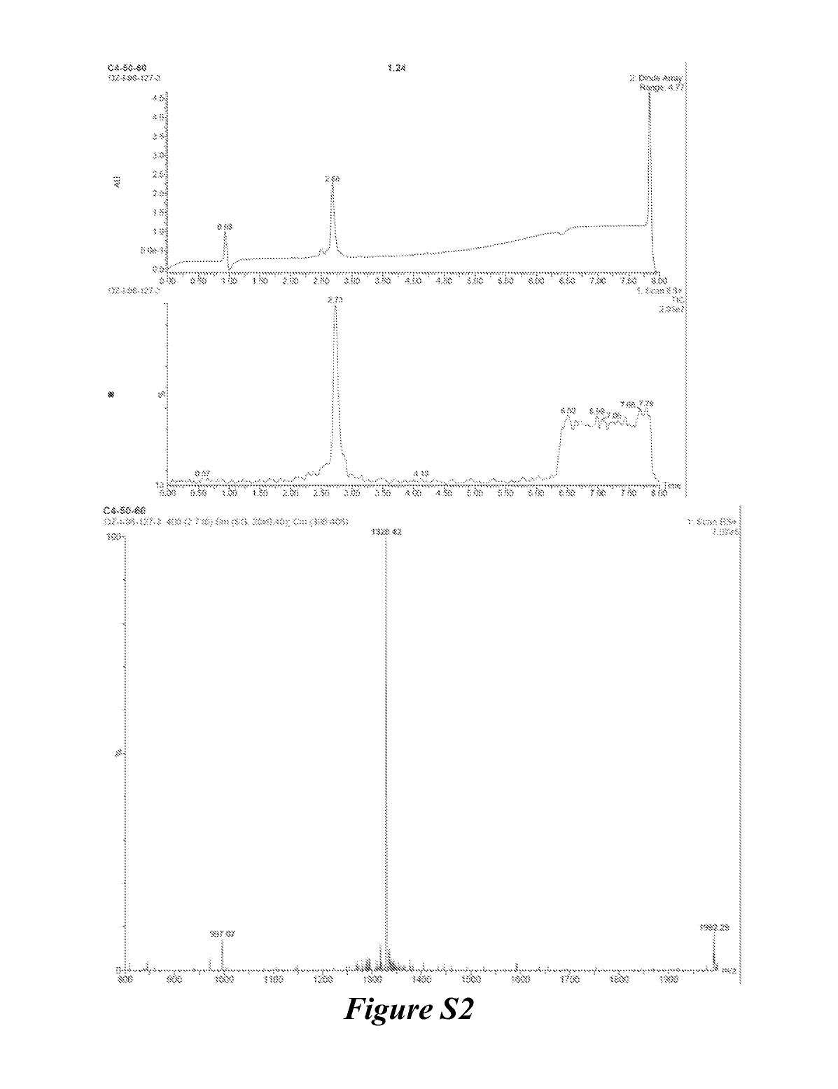

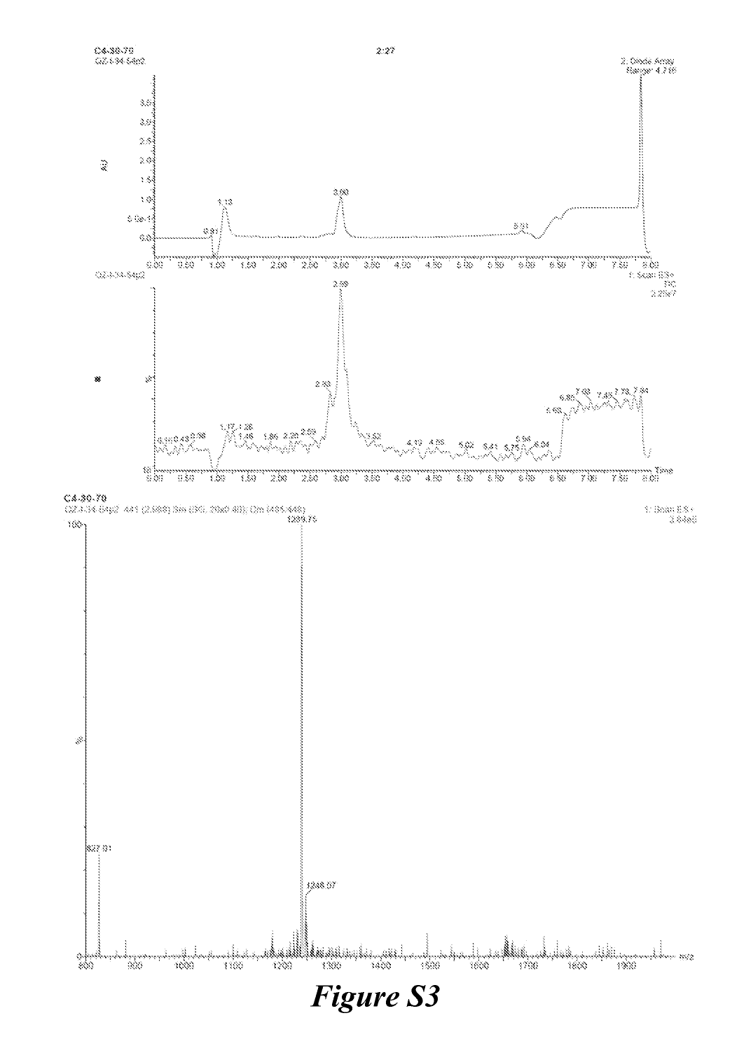

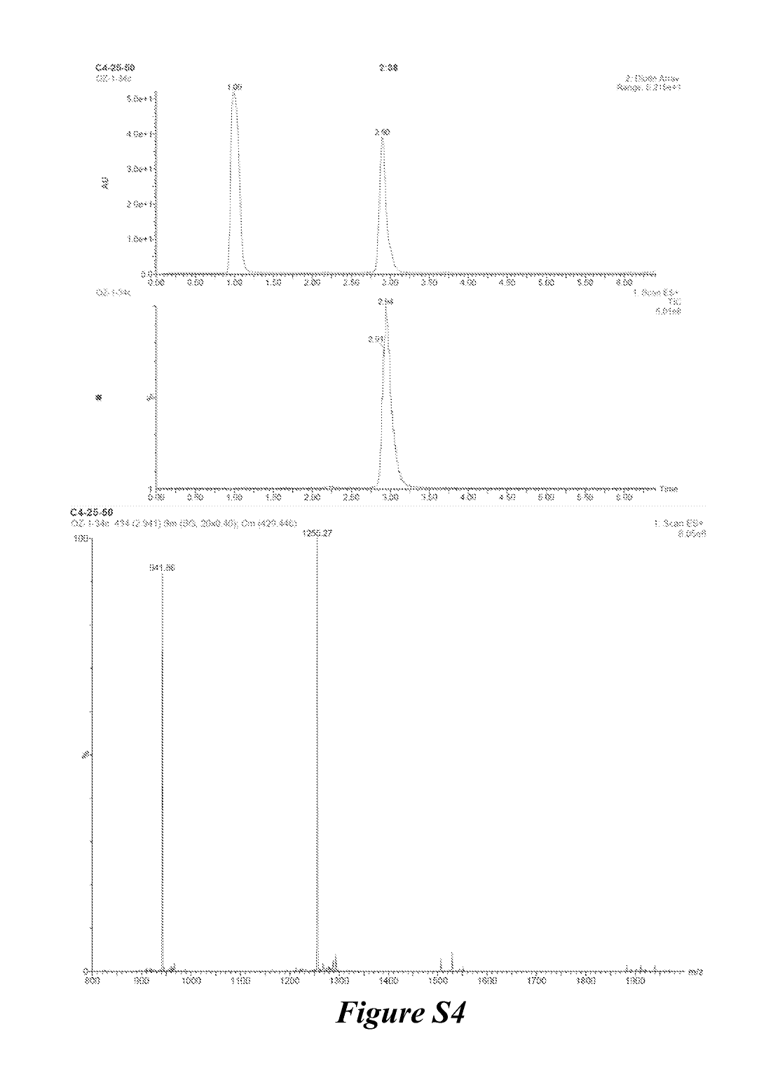

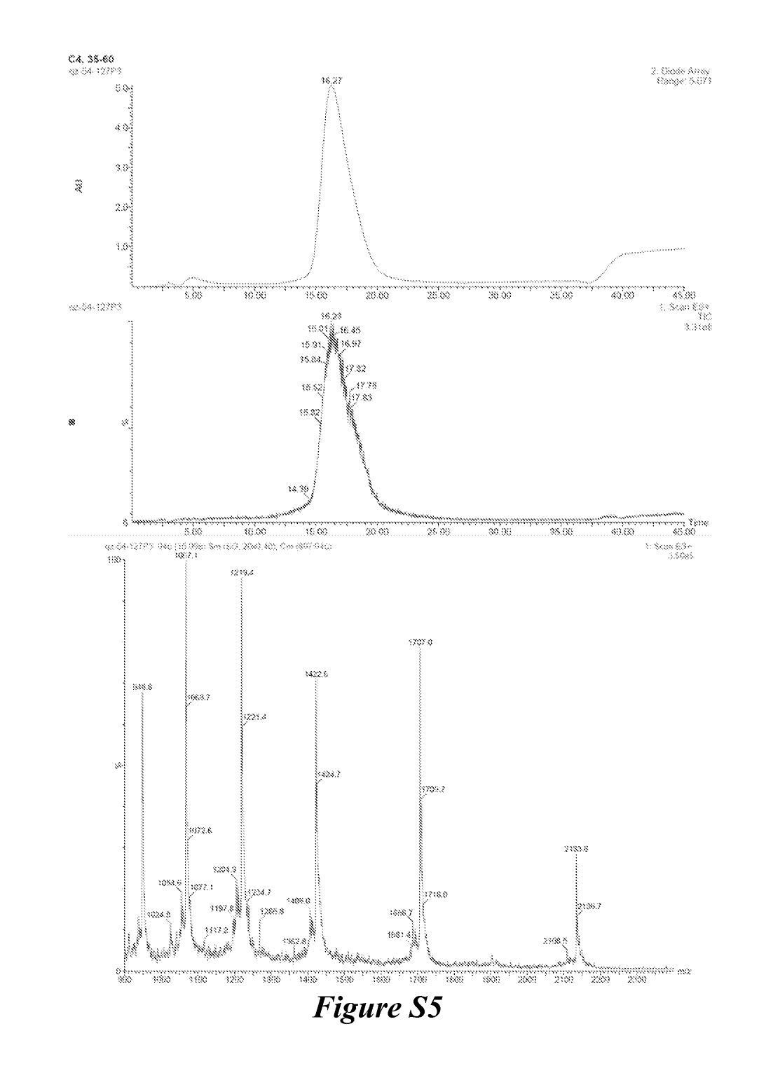

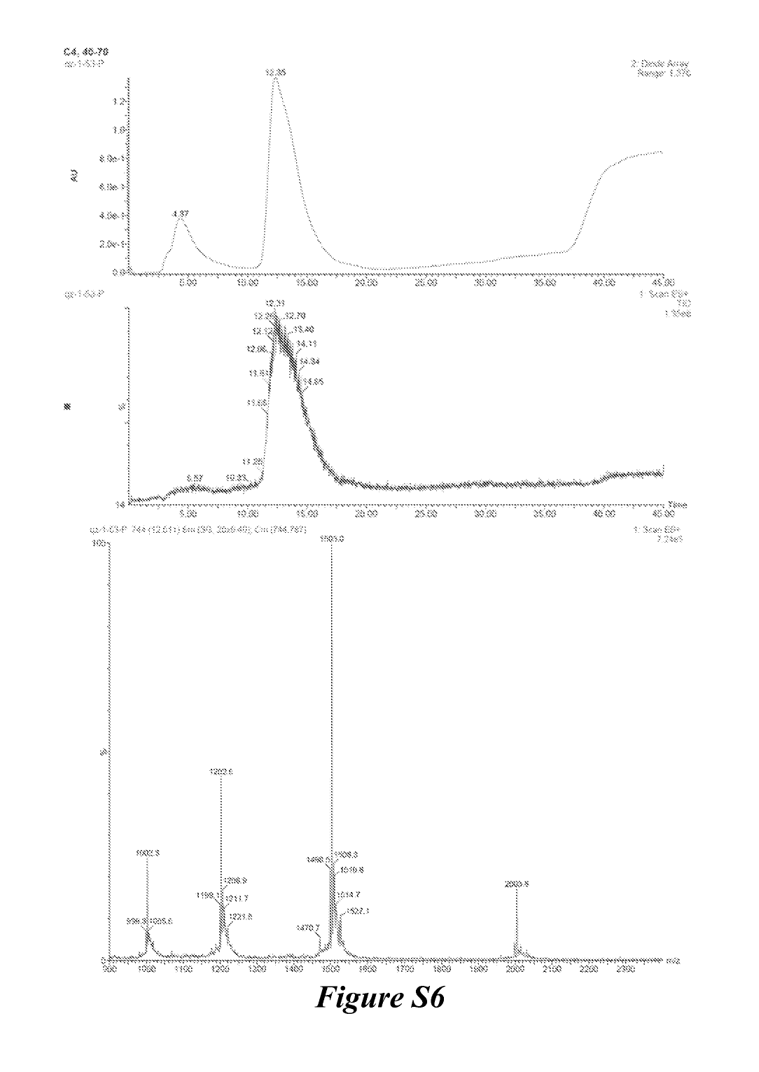

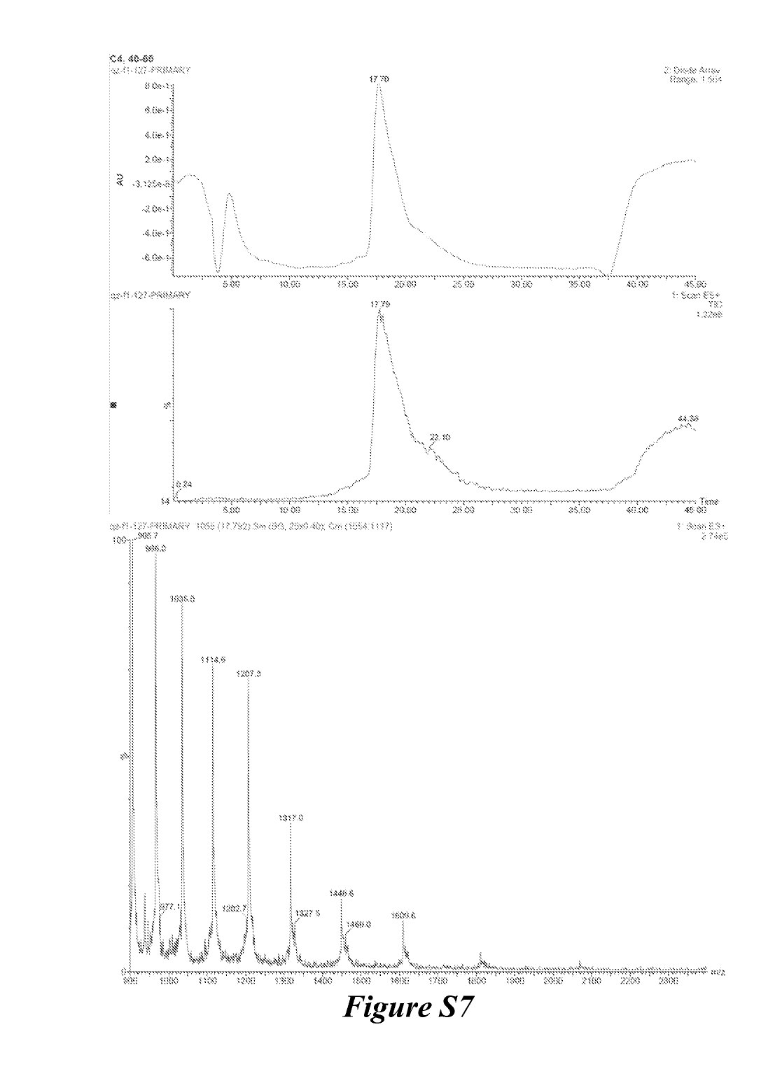

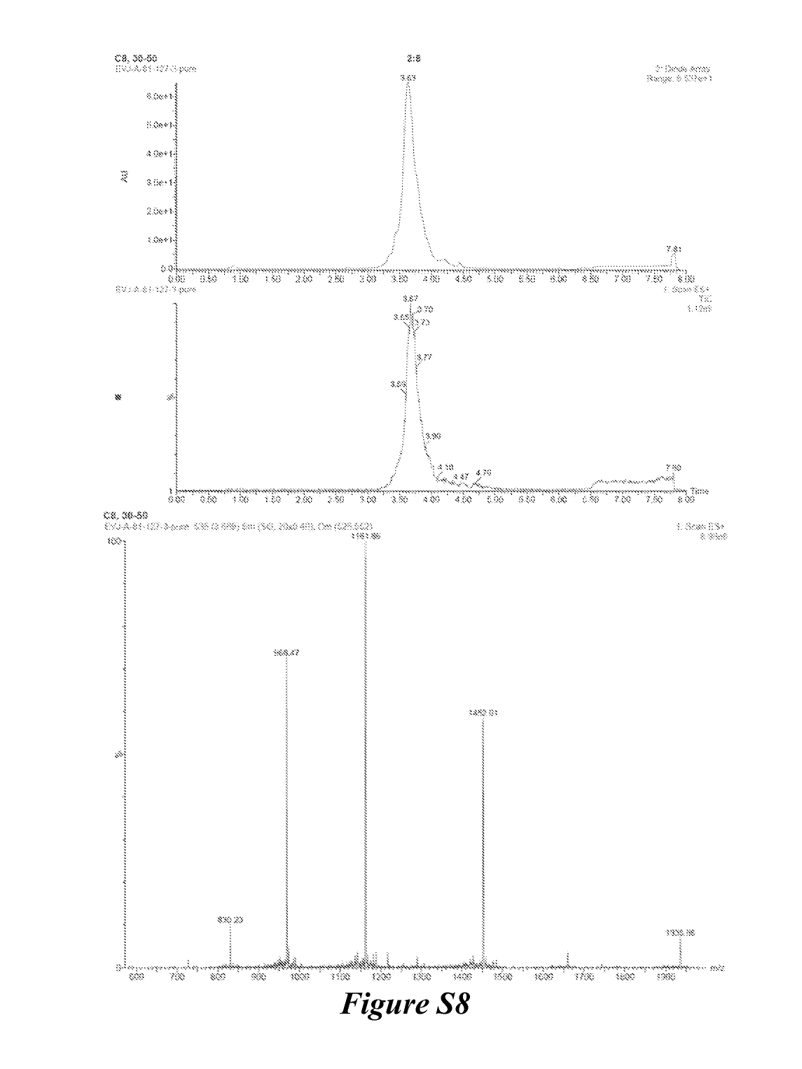

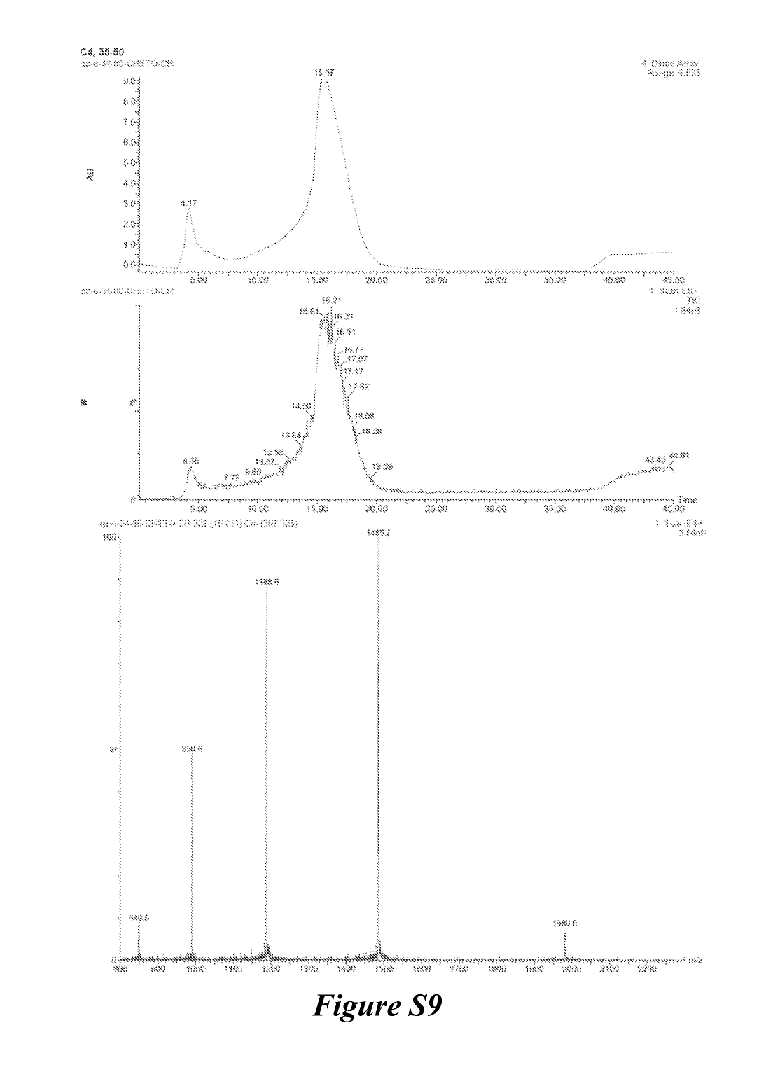

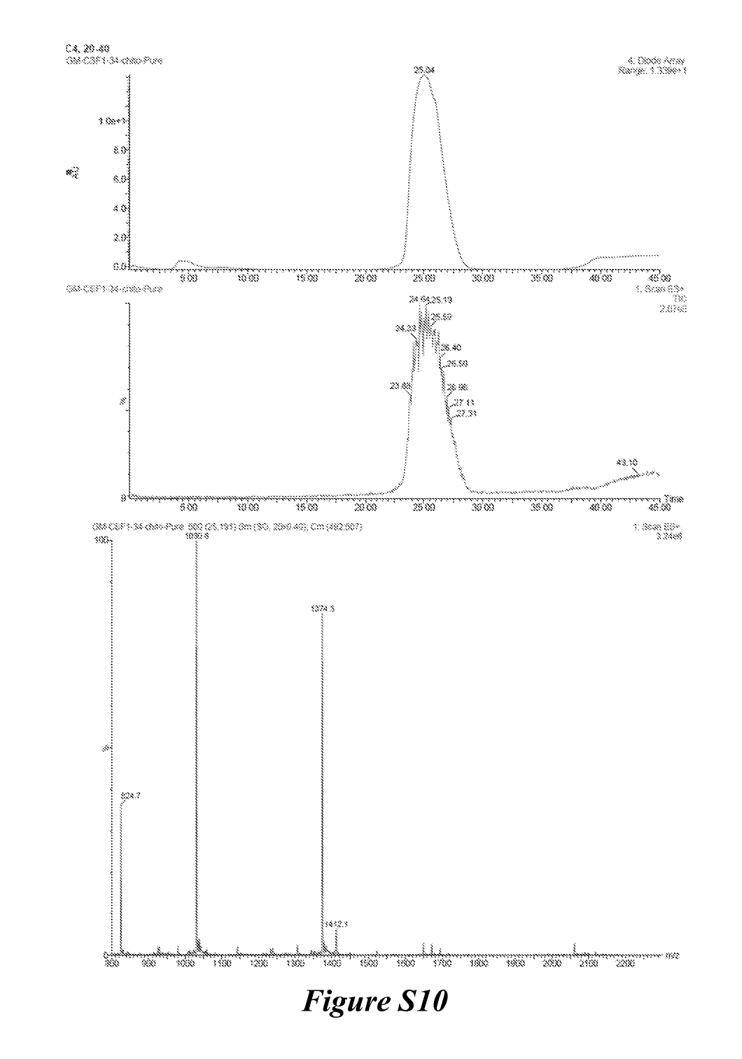

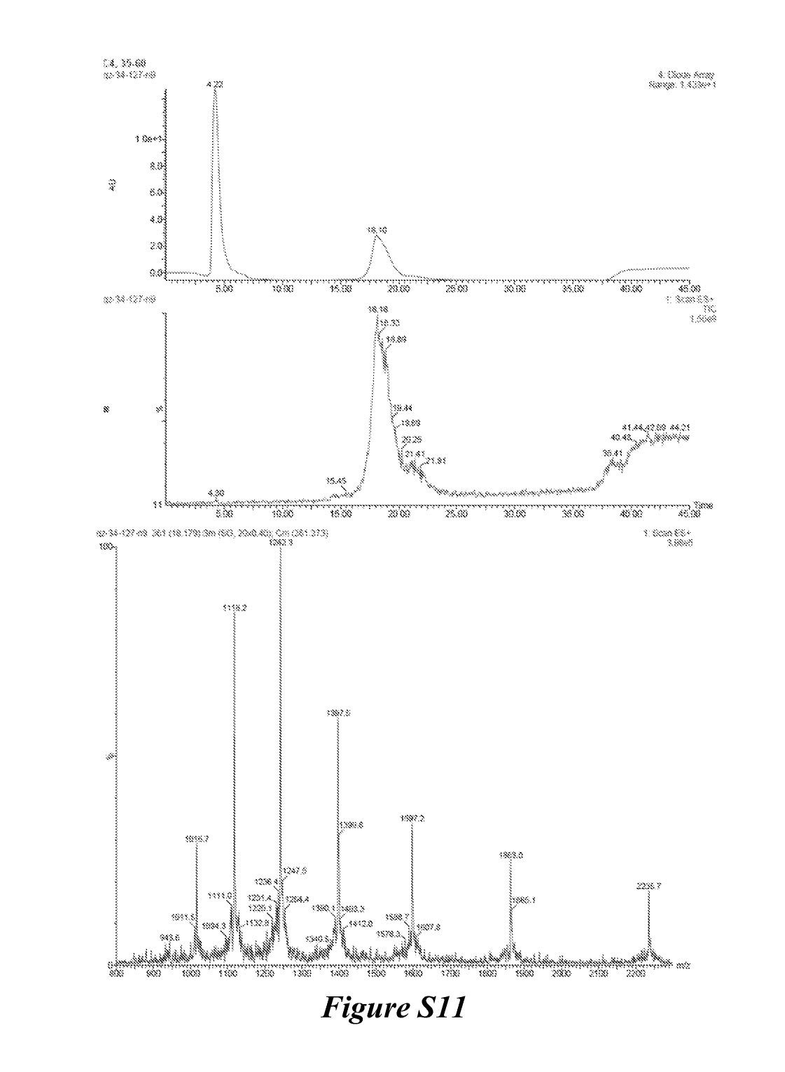

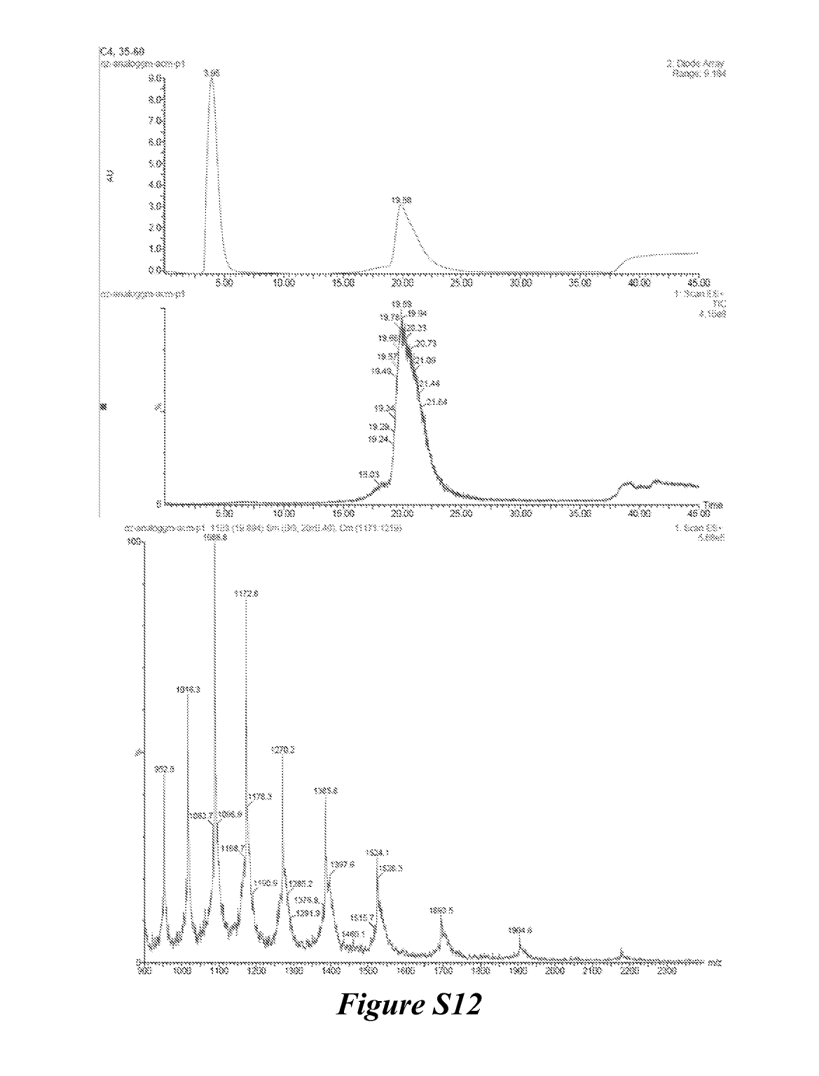

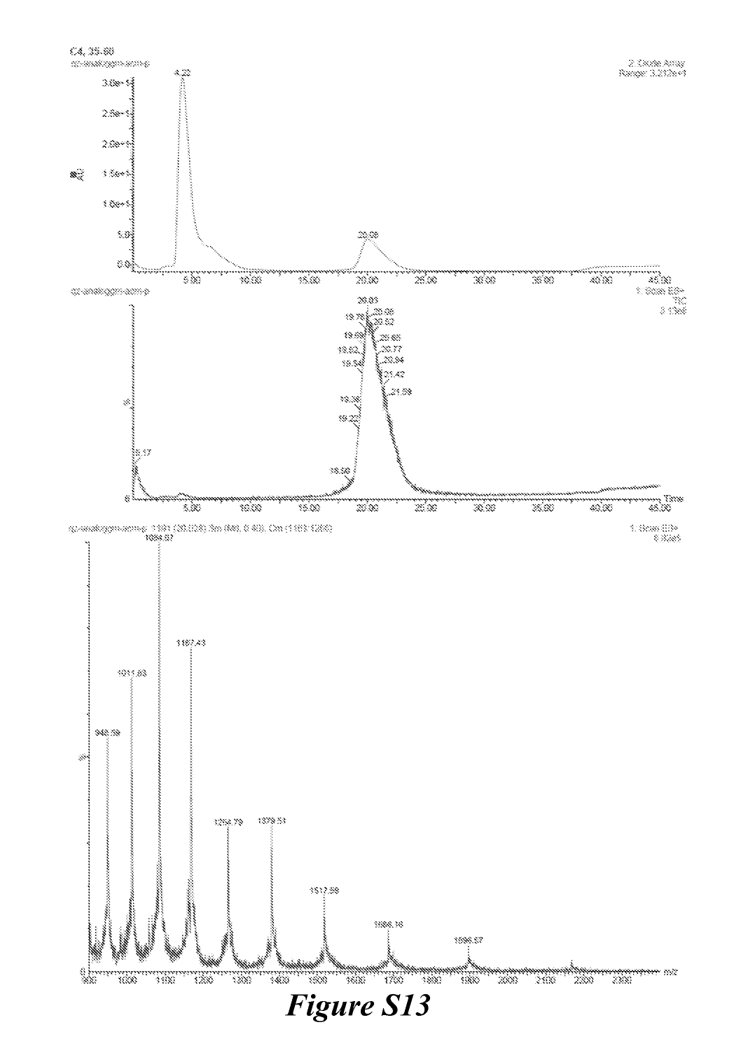

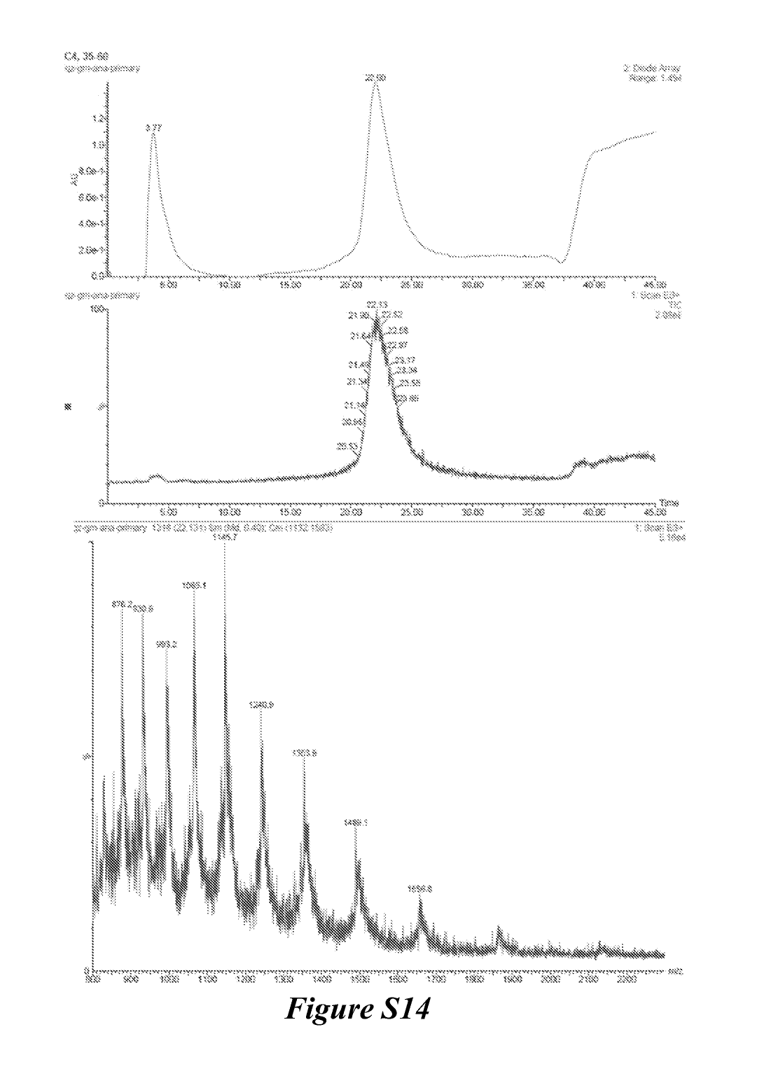

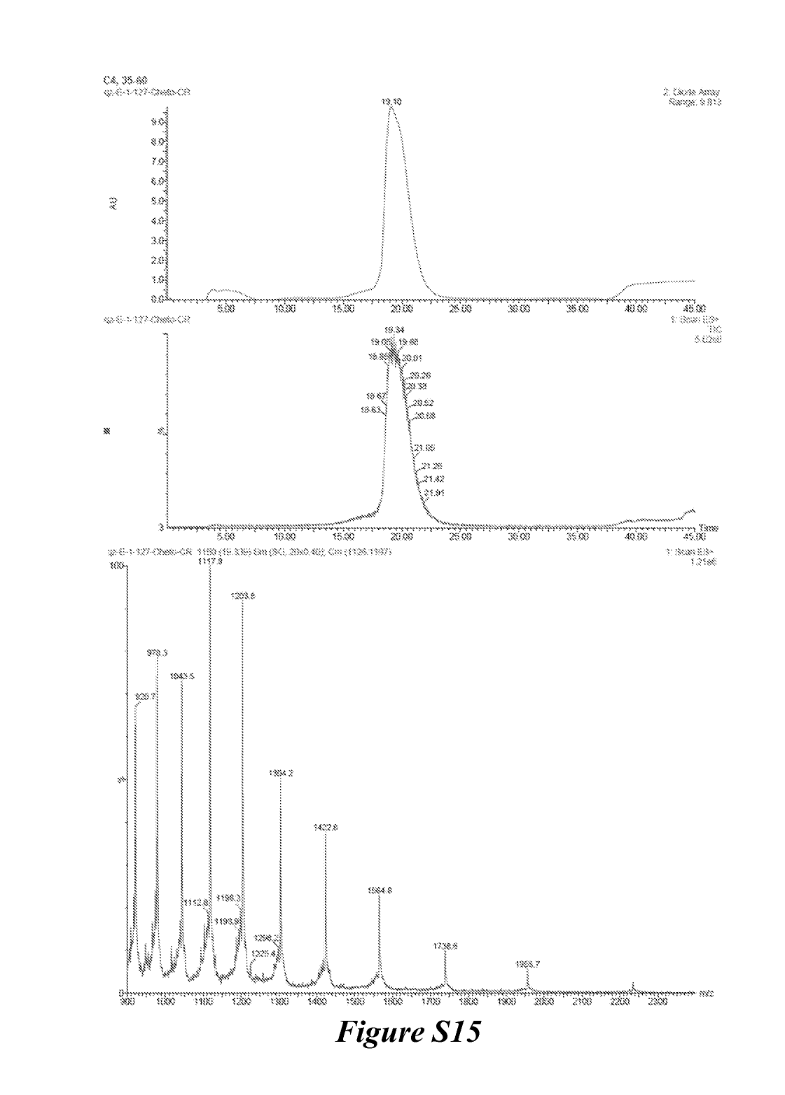

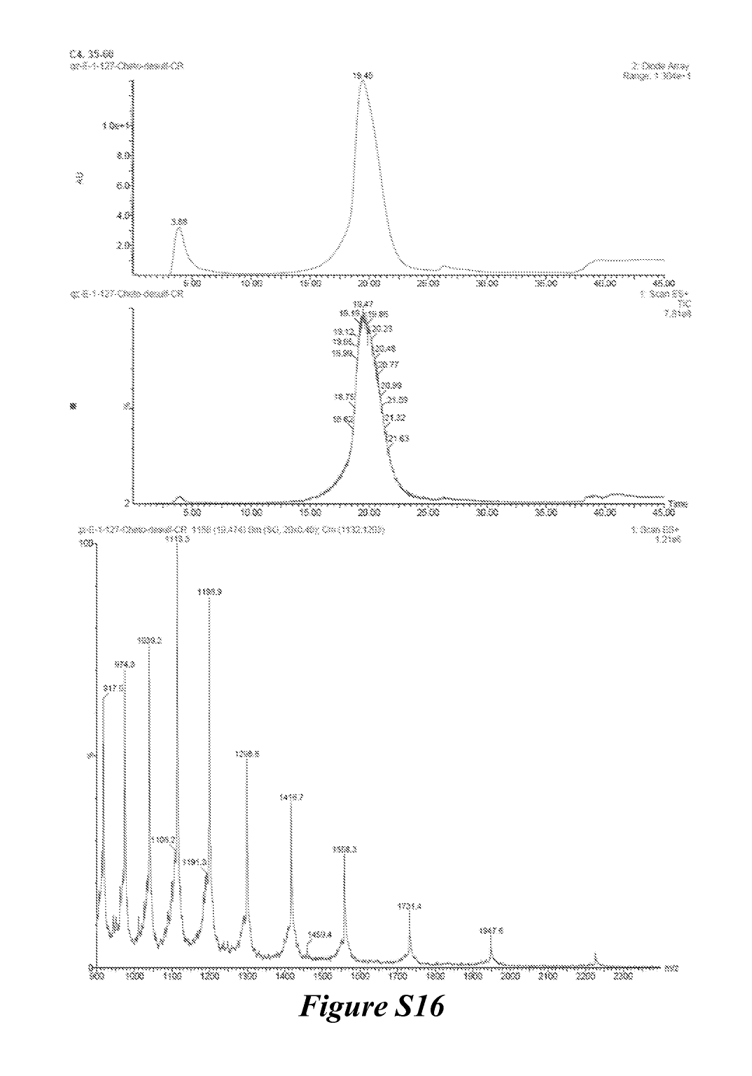

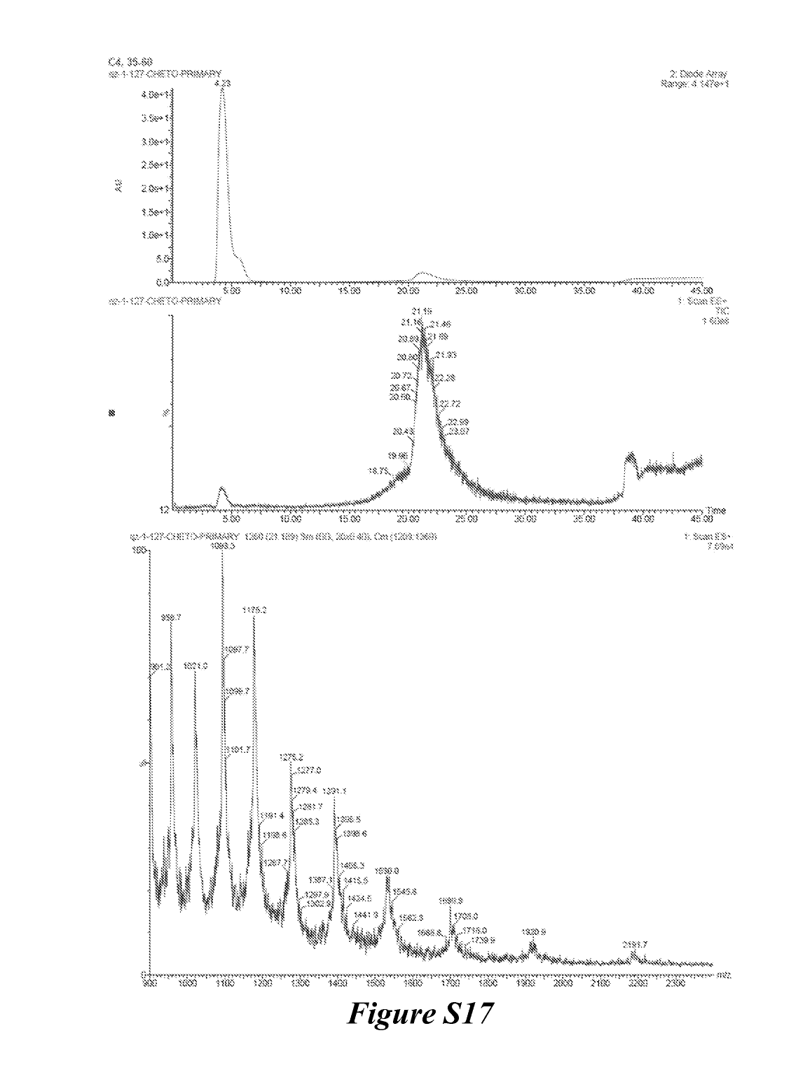

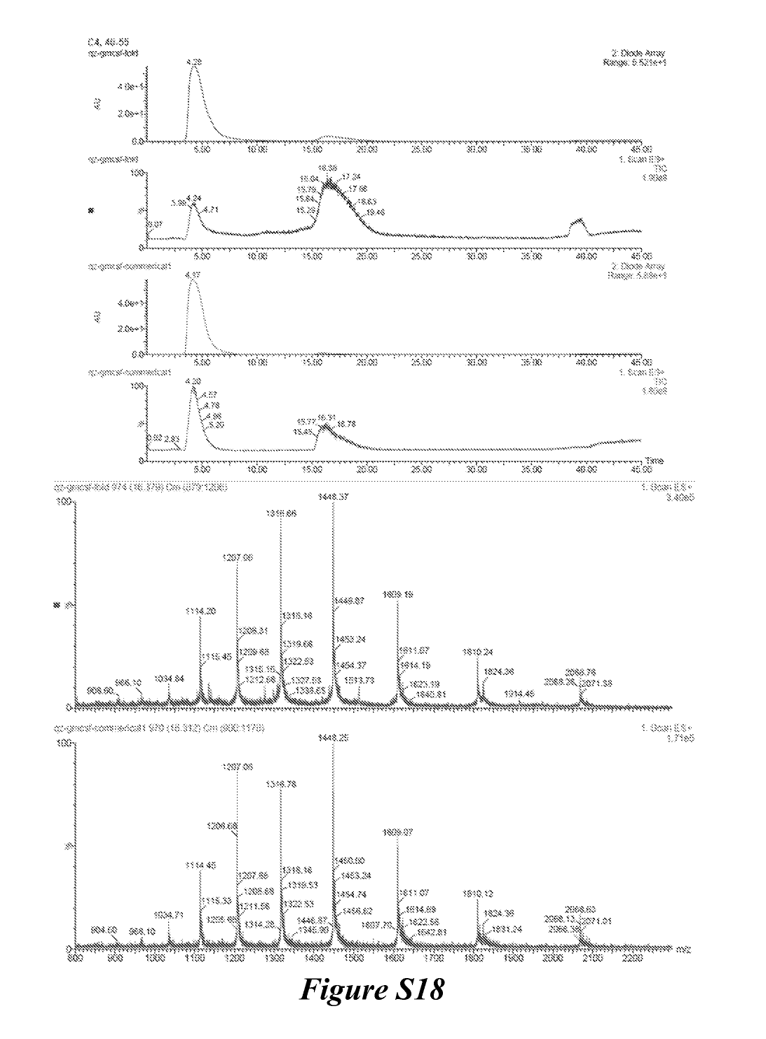

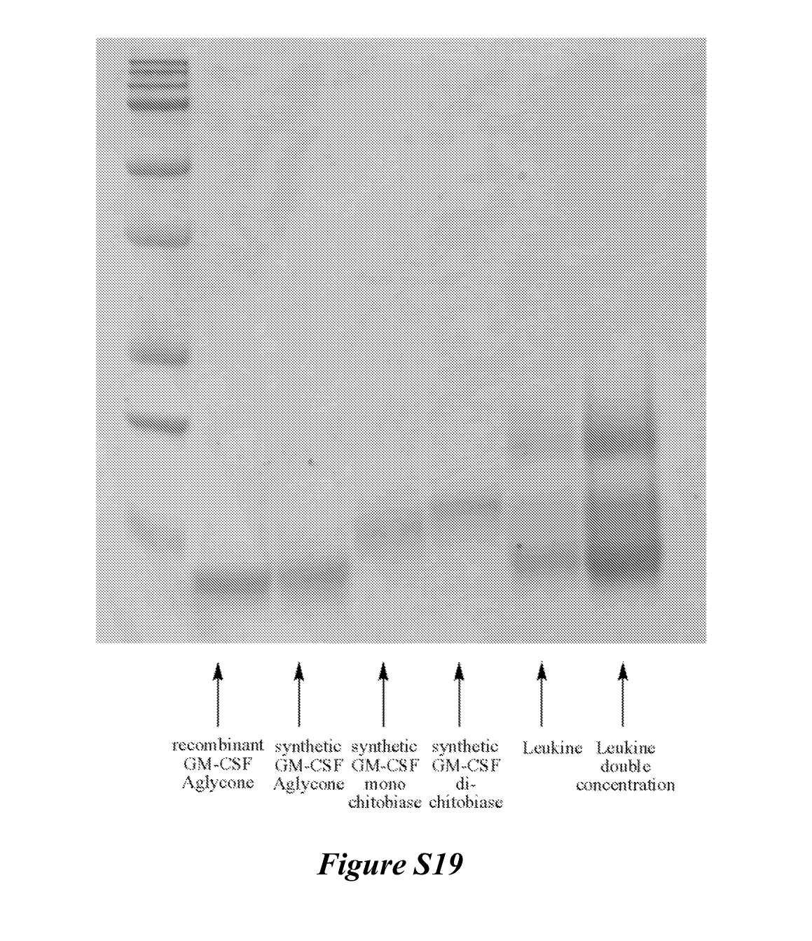

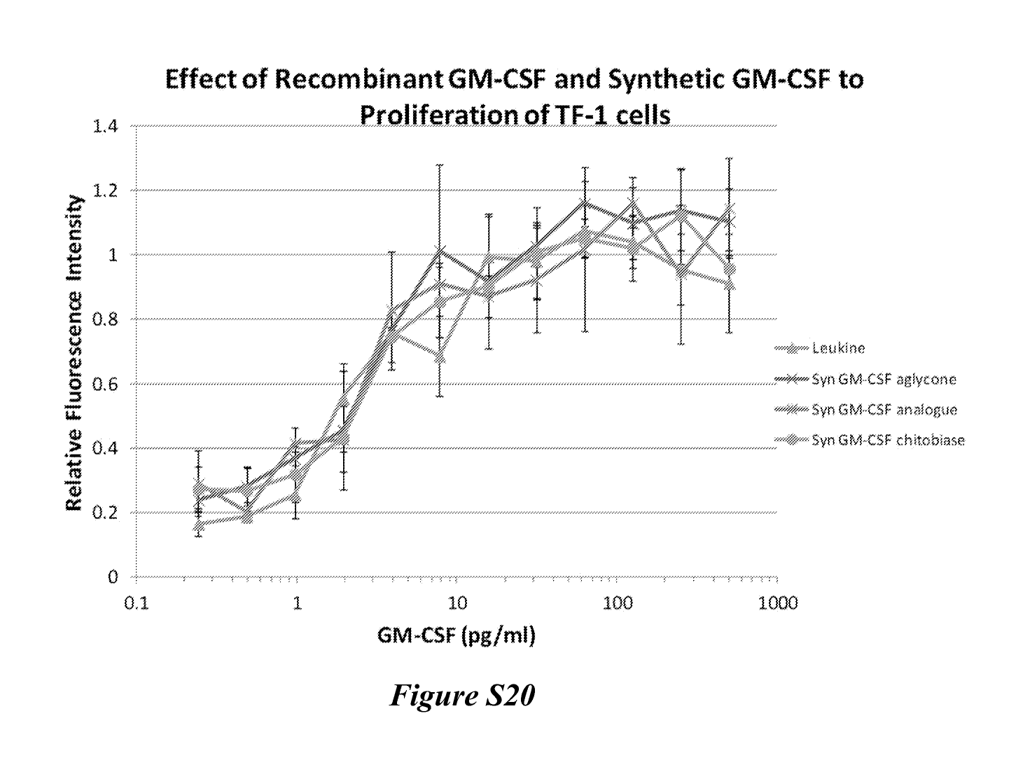

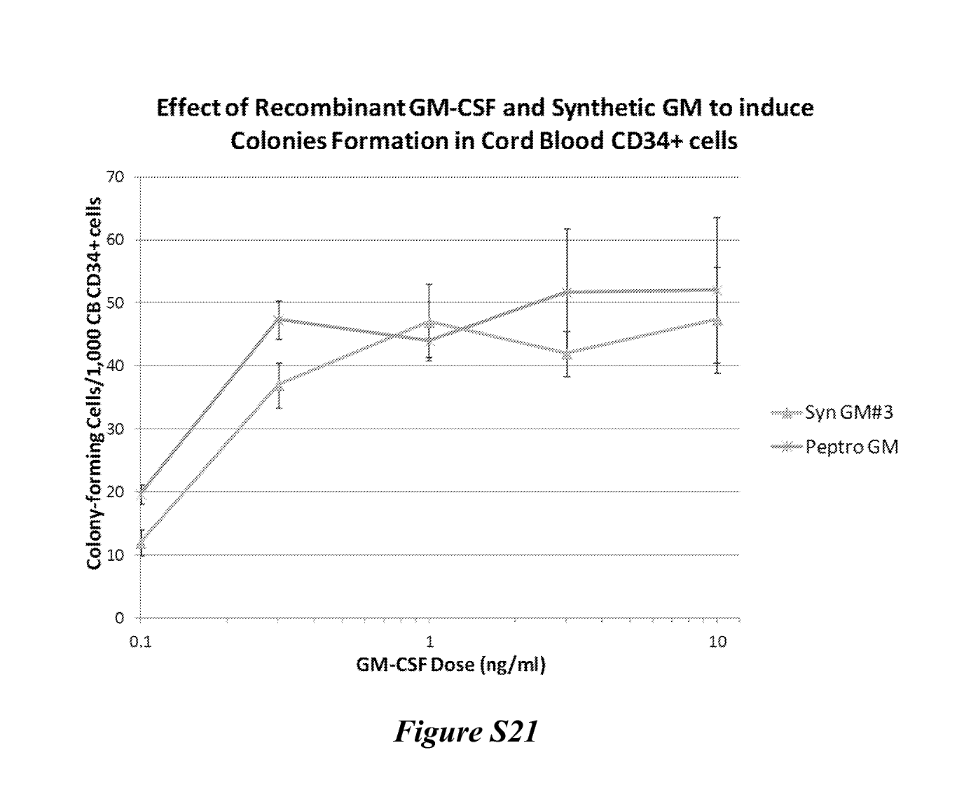

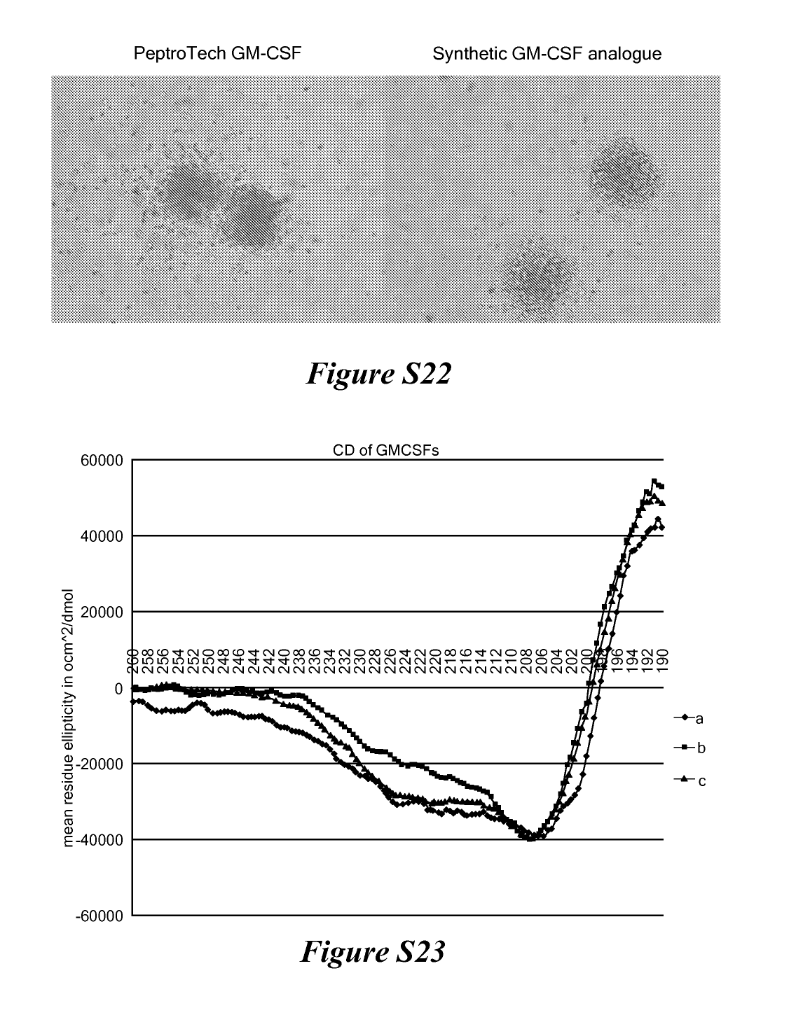

Due to a low yield observed in the final NCL reaction for the non-glycosylated GM-CSF synthesis, an alternative strategy was sought given the relative preciousness of the glycopeptide pieces. One strategy for preparation of glycosylated GM-CSF (Nr) is depicted in FIG. 4. This strategy envisions that the glycosylated peptide could be assembled from three fragments (I-III). Glycopeptide fragments I and II would be prepared with N-linked carbohydrates installed at the native positions (Asn²⁷ and Asn³⁷), and the connection between the fragments would be exclusively alanine ligations. In this scenario however, all of the cysteine residues would not participate in ligations and were protected by acetamidomethyl (ACM) functional groups.

Beginning a glycosylated GM-CSF synthesis with the preparation of Fragment II (Scheme 2), glycopeptide II was accessed through multiple-step maneuvers. SPPS provided fully protected polypeptide 12 with an allyl-protected Asp³⁷ residue, which was selectively removed using a catalytic amount of palladium (0) to generate 13. Then, Lansbury aspartylation of 13 with chitobiose cleanly afforded glycopeptide 14 (P. Wang, B. Aussedat, Y. Vohra, S. J. Danishefsky, Angew. Chem. 2012, Volume 51, Issue 46, pages 11571-11575). Finally, global deprotection of peptide 14 liberated target Fragment II (15). Fragment III (16) was successfully prepared by SPPS employing the oxyma pure/piperidine deblocking protocol. NCL of Fragments II and III followed by Thz removal provided intermediate 17 in good yield. The final coupling of glycopeptide 17 with Fragment I (18) generated the main construct of glycosylated GM-CSF 19 in excellent yield. In the penultimate step the two cysteine residues (Cys, Cys) were reduced to their native alanine forms by metal-free desulfurization of main construct 19, successfully proving the desired ACM GM-CSF 20. The ACM protecting groups were removed by AgOAc (Fujii N, Otaka A, Watanabe T, Okamachi A, Tamamura H, Yajima H, Inagaki Y, Nomizu M, Asano K (1989) J Chem Soc Chem Commun, 283-284) followed by acidic DTT quenching to produce denatured GM-CSF 21 in good yield.

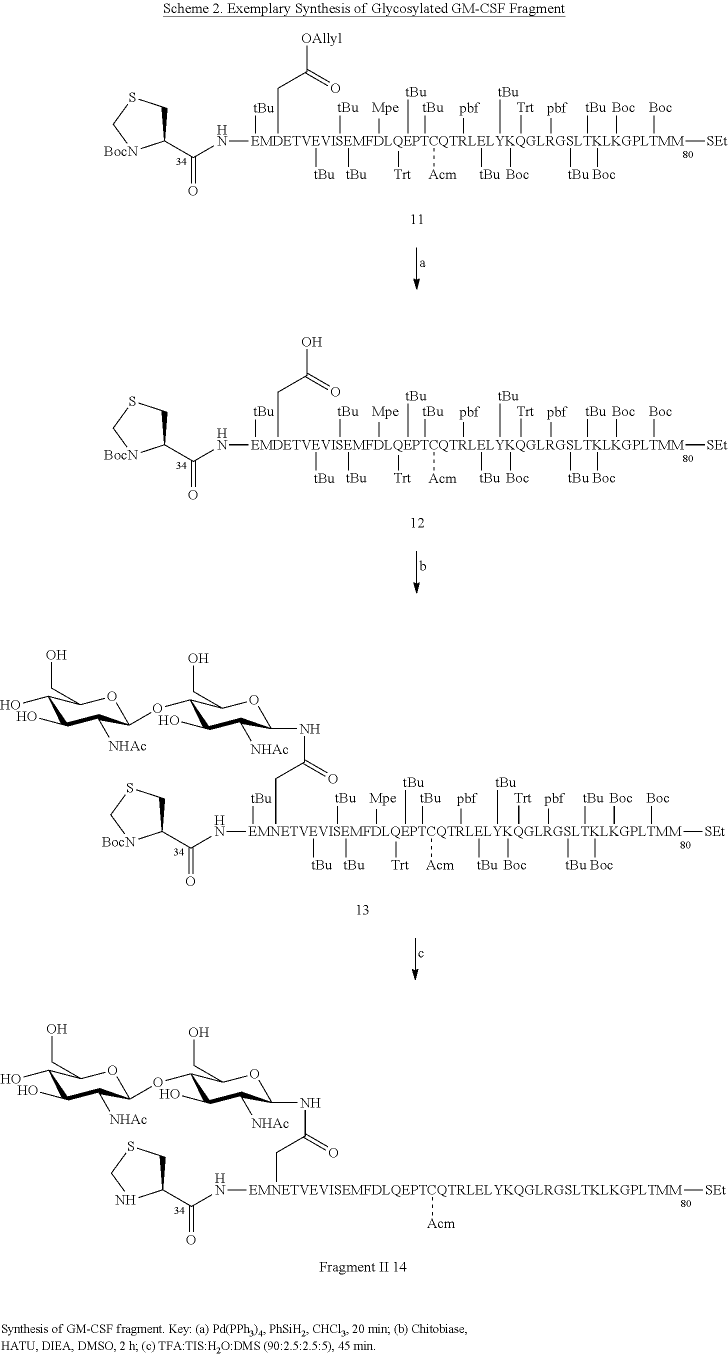

In order to further explore the effect of N-glycosylation on the peptide backbone, a third analogue of glycosylated GM-CSF with a single N-glycan (Asn37) was prepared (Scheme 3a-b). The primary construct 22 was obtained via identical ligation conditions to the glycosylated one.

Finally, the folding glycosylated GM-CSF 21 and its analogue 23 generated native protein, which were subjected to further biological evaluation (Scheme 3a-b).

In addition to the polypeptides described above, the present disclosure also contemplates variants of GM-CSF, including but not limited to variants that possess advantageous properties relevant to stability, toxicity, and bioavailability. The present disclosure enables the production of such variants through the provision of the synthetic pathways described herein. In some embodiments, GM-CSF can be modified to impart better resistance to in vivo enzymes such as peptidases. Such modifications are known in the art and include the use of unnatural amino acids, leader sequences, and/or glycosylation.

In some embodiments, a provided polypeptide is a GM-CSF prodrug. In certain embodiments, a GM-CSF prodrug has a C- or N-terminus chain extended by an artificial peptide or modified peptide sequence, whereupon suitable bioactivation, the artificial sequence is cleaved to restore the bioactivity of GM-CSF or one of its improved congeners. In some embodiments, such prodrugs provide greater control and flexibility of the pharmacokinetic performance of GM-CSF. Methods of making such prodrugs are known in the art, and include, for example, those described by Stella, Annu. Rev. Pharmacol. Toxicol. 1993. 32:521-44, and Moreira, Molecules 2007, 12, 2484-2506.

The present disclosure describes the successful synthesis of GM-CSF and its analogues. The synthetically pure products all demonstrate great in vitro activities compared to commercially available samples. The established route to access the glycoprotein utilized cysteine and alanine NCLs paired with mild organo-desulfurization. With the promising results obtained in this study, further in vivo investigations of the O-linked carbohydrates as well as more complicated N-linked carbohydrates installed on the GM-CSF peptidyl backbone are underway.

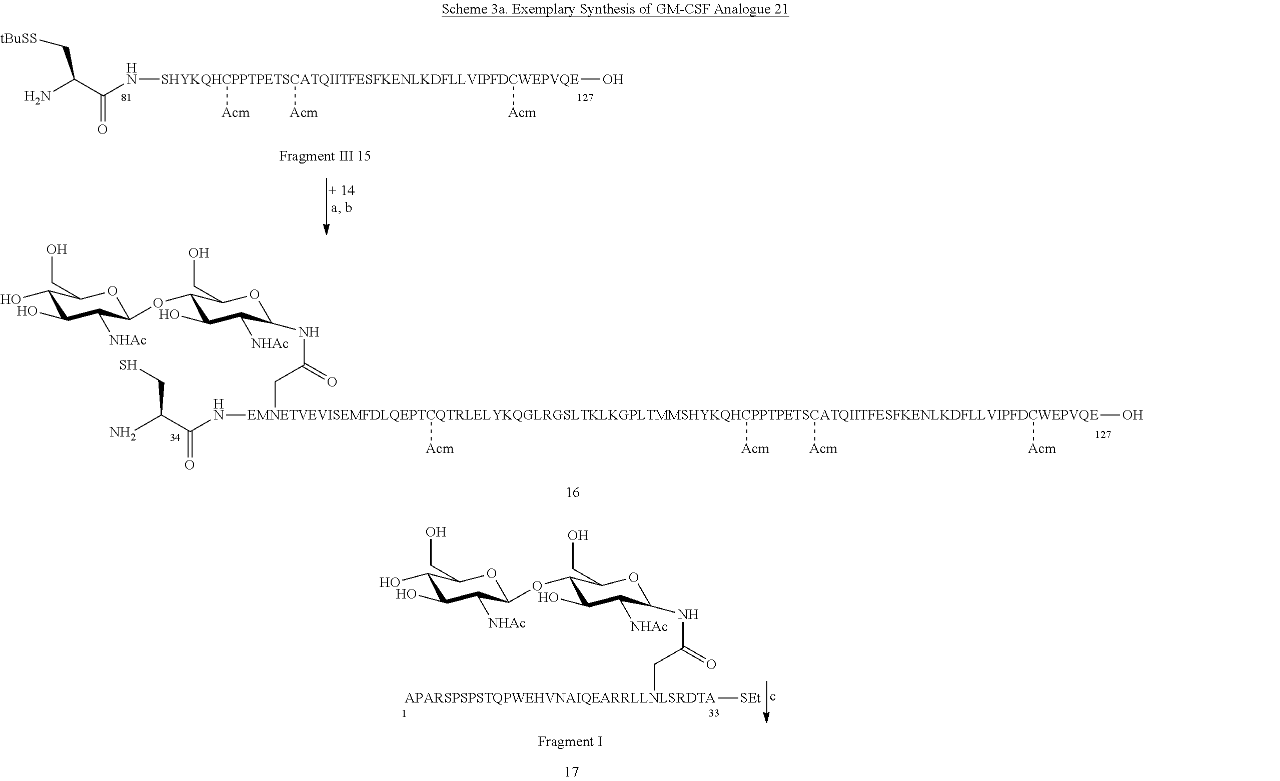

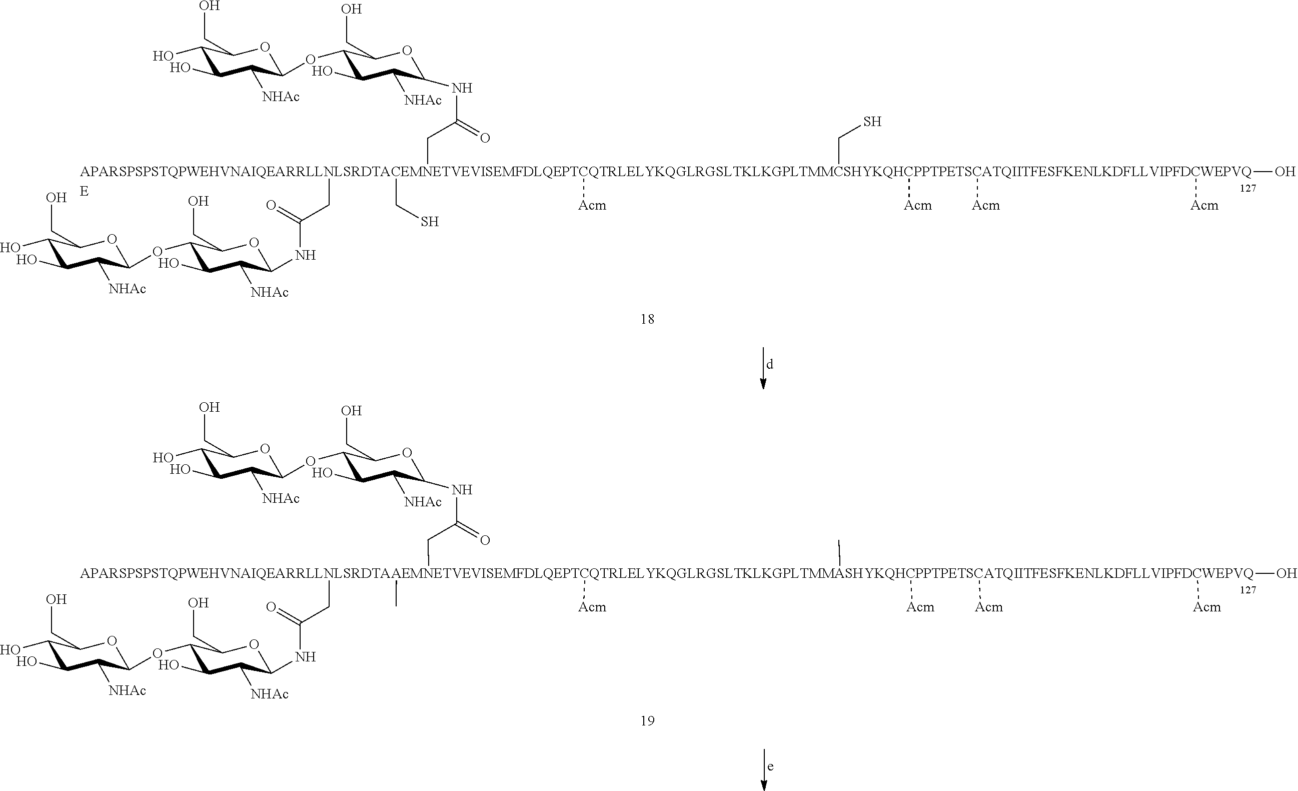

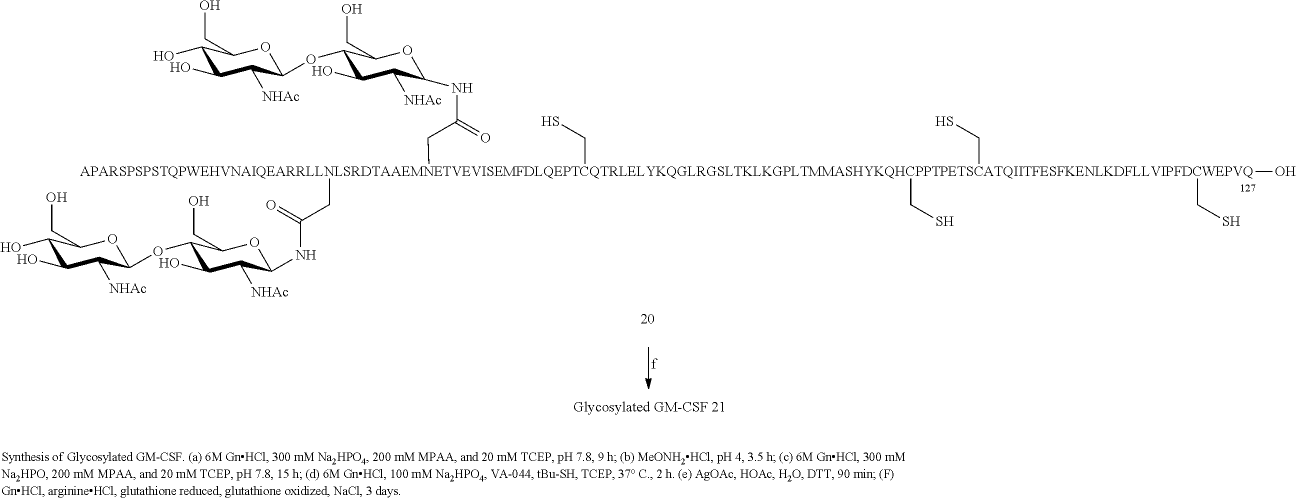

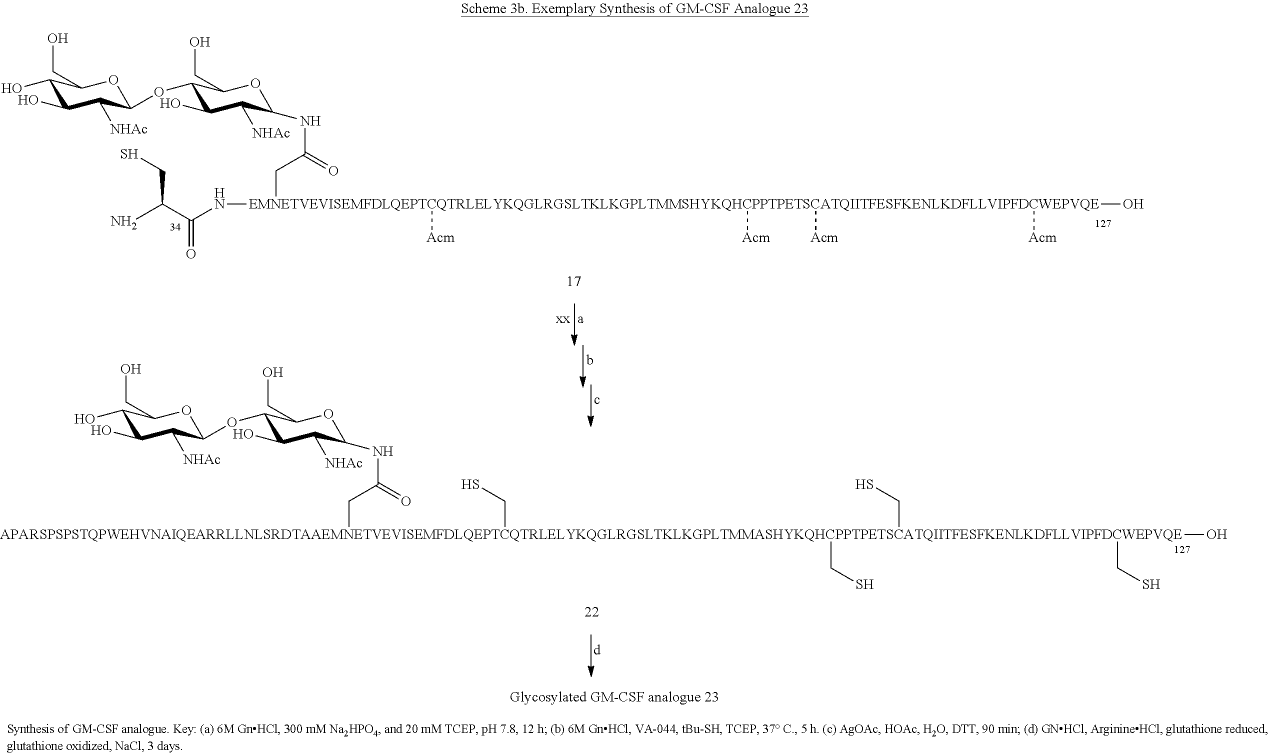

In certain embodiments, the present invention provides a method of preparing GM-CSF, the method comprising the step of:

ligating to one another a set of fragments of a polypeptide whose amino acid sequence includes a sequence that:
a) is identical to that of:

(SEQ ID NO: 1)
Ala-Pro-Ala-Arg-Ser-Pro-Ser-Pro-Ser-Thr-Gln-Pro-
Trp-Glu-His-Val-Asn-Ala-Ile-Gln-Glu-Ala-Arg-Arg-
Leu-Leu-Asn-Leu-Ser-Arg-Asp-Thr-Ala-Ala-Glu-Met-
Asn-Glu-Thr-Val-Glu-Val-Ile-Ser-Glu-Met-Phe-Asp-
Leu-Gln-Glu-Pro-Thr-Cys-Leu-Gln-Thr-Arg-Leu-Glu-
Leu-Tyr-Lys-Gln-Gly-Leu-Arg-Gly-Ser-Leu-Thr-Lys-
Leu-Lys-Gly-Pro-Leu-Thr-Met-Met-Ala-Ser-His-Tyr-
Lys-Gln-His-Cys-Pro-Pro-Thr-Pro-Glu-Thr-Ser-Cys-
Ala-Thr-Gln-Ile-Ile-Thr-Phe-Glu-Ser-Phe-Lys-Glu-
Asn-Leu-Lys-Asp-Phe-Leu-Leu-Val-Ile-Pro-Phe-Asp-
Cys-Trp-Glu-Pro-Val-Gln-Glu

which set of fragments includes fragments whose amino acid sequence corresponds to amino acid residues 1-33, 34-53, 34-80, 54-95, 81-127, or 96-127 of SEQ ID NO: 1, so that a homogenously glycosylated GM-CSF polypeptide is generated. In some embodiments, each molecule of GM-CSF or fragment thereof has the same glycosylation pattern, and for a given glycosylation site each molecule of GM-CSF or fragment thereof has the same glycan.

### EXEMPLIFICATION

### Materials and Methods

All commercially available materials (Aldrich®, Fluka®, Novabiochem®) were used without further purification. 2,2′-Azobis[2-(2-imidazolin-2-yl)propane]dihydrochloride (VA044) was purchased from Wako Pure Chemical Industries. HATU was purchased from Genscript® (Piscataway, N.J.). Bond-Breaker® solution was purchased from ThermoScientific®. Chitobiose octaacetate was purchased from Toronto Research Chemicals Inc. All solvents were reagent grade or HPLC grade (Fisher®) Anhydrous THF, diethyl ether, CH₂Cl₂, toluene, and benzene were obtained from a dry solvent system (passed through column of alumina) and used without further drying. All reactions were performed under an atmosphere of pre-purified dry Ar(g). NMR spectra (¹H and ¹³C) were recorded on a Bruker Advance II 600 MHz or Bruker Advance DRX-500 MHz, referenced to TMS or residual solvent. Low-resolution mass spectral analyses were performed with a JOEL JMS-DX-303-HF mass spectrometer or Waters Micromass ZQ mass spectrometer. Analytical TLC was performed on E. Merck silica gel 60 F254 plates and flash column chromatography was performed on E. Merck silica gel 60 (40-63 mm). Yields refer to chromatographically pure compounds.

HPLC:

All separations involved a mobile phase of 0.05% TFA (v/v) in water (solvent A)/0.04% TFA in acetonitrile (solvent B). Analytical LC-MS analyses were performed using a Waters 2695 Separations Module and a Waters 996 Photodiode Array Detector equipped with Varian Microsorb 100-5, C18 150×2.0 mm, and Varian Microsorb 300-5, C4 250×2.0 mm columns at a flow rate of 0.2 mL/min.

UPLC-MS analyses were performed using a Waters Acquity™ Ultra Preformance LC system equipped with Acquity UPLC® BEH C18, 1.7 μl, 2.1×100 mm, Acquity UPLC® BEH C8, 1.7 μl, 2.1×100 mm, Acquity UPLC® BEH 300 C4, 1.7 μl, 2.1×100 mm columns at a flow rate of 0.3 mL/min.

Preparative separations were performed using a Ranin HPLC solvent delivery system equipped with a Rainin UV-1 detector and Agilent Dynamax reverse phase HPLC column (Microsorb 100-8 C18 (250×21.4 mm), or Microsorb 300-5 C8 (250×21.4 mm), or Microsorb 300-5 C4 (250×21.4 mm)) at a flow rate of 16.0 mL/min.

### General Procedures:

A: Solid Phase Peptide Synthesis Using Fmoc-Strategy.

Automated peptide synthesis was performed on an Applied Biosystems Pioneer continuous flow peptide synthesizer. Peptides were synthesized under standard automated Fmoc protocols. The deblock mixture was a mixture of 100:2:2 of DMF/piperidine/DBU. The following Fmoc amino acids and pseudoproline dipeptides from Novabiochem® were employed: Fmoc-Ala-OH, Fmoc-Arg(Pbf)OH, Fmoc-Asn(Trt)-OH, Fmoc-Asp(OtBu)-OH, Boc-Thz-OH, Fmoc-Glu(OtBu)-OH, Fmoc-Gln(Trt)-OH, Fmoc-Gly-OH, Fmoc-His(Trt)-OH, Fmoc-Ile-OH, Fmoc-Leu-OH, FmocLys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Val-OH, Fmoc-Asp(OtBu)-Ser(ψ^{Me,Me}Pro)-OH, Fmoc-Asp(OtBu)-Thr(ψ^{Me,Me}Pro)-OH, Fmoc-Ile-Ser(ψ^{Me,Me}Pro)-OH, Fmoc-Ile-Thr(ψ^{Me,Me}Pro)-OH, Fmoc-Leu-Ser(ψ^{Me,Me}Pro)-OH, Fmoc-Leu-Thr(ψ^{Me,Me}Pro)-OH, Fmoc-Ser(tBu)-Ser(ψ^{Me,Me}Pro)-OH, Fmoc-Tyr(tBu)-Ser(ψ^{Me,Me}Pro)-OH, Fmoc-Tyr(tBu)-Thr(ψ^{Me,Me}Pro)-OH, Fmoc-Val-Ser(ψ^{Me,Me}Pro)OH.

Upon completion of the automated synthesis on a 0.05/0.10 mmol scale, the peptide resin was washed into a peptide cleavage vessel with DCM. The resin cleavage was performed with TFA/H₂O/triisopropylsilane (95:2.5:2.5 v/v) solution or DCM/AcOH/TFE (8:1:1 v/v) for 45 min (×2). The liquid was blown off with nitrogen. The oily residue was extracted with diethyl ether and centrifuged to give a white pellet. After the ether was decanted, the solid was lyophilized or purified for further use.

B: Preparation of Peptidyl Esters.

The fully protected peptidyl acid (1.0 equiv) cleaved from resin using DCM/TFE/AcOH (8:1:1, v/v), and the amino acid ester hydrochloride (3.0 equiv) were dissolved in CHCl₃ and cooled to −10° C. HOOBt (3.0 equiv) and EDCI (3.0 equiv) were then added. The reaction mixture was stirred at room temperature for 4 h. The solvent was gently blown off by a nitrogen stream and the residue was washed with H₂O/AcOH (95:5, v/v). After centrifugation, the pellet was dissolved in TFA/H₂O/TIS (95:2.5:2.5) and stirred at room temperature for 1 h. The solvent was removed and the residue was triturated with cold ether. The resulting solid was dissolved in MeCN/H₂O/AcOH (47.5:47.5:5, v/v) for further analysis and purification.

C: Kinetic Native Chemical Ligation with Peptidyl Thiophenol Ester.

N-terminal peptide ester (1.0 equiv) and C-terminal peptide (1.0 equiv) were dissolved in ligation buffer (6 M Gnd.HCl, 300 mM Na₂HPO₄, 20 mM TCEP.HCl, pH 6.9˜7.0). The resulting solution was stirred at room temperature, and monitored using LC-MS. The reaction was quenched with MeCN/H₂O/AcOH (47.5:47.5:5) and purified by HPLC.

D: Native Chemical Ligation with Peptidyl Alkylthio Ester.

N-terminal peptide ester (1.0 equiv) and C-terminal peptide (1.0 equiv) were dissolved in ligation buffer (6 M Gnd.HCl, 300 mM Na₂HPO₄, 20 mM TCEP.HCl, 200 mM 4-mercaptophenylacetic acid (MPAA), pH 7.7˜7.8). The resulting solution was stirred at room temperature, and monitored using LC-MS. The reaction was quenched with MeCN/H₂O/AcOH (47.5:47.5:5) and purified by HPLC.

E: Metal-Free Dethiylation.

To a solution of the purified ligation product in 0.2 ml of degassed buffer (6 M Gnd.HCl, 200 mM Na₂HPO₄) was added 0.2 ml of 0.5 M Bond-Breaker® TCEP solution (Pierce), 0.05 ml of 2-methyl-2-propanethiol and 0.1 ml of radical initiator VA044 (0.1 M in H₂O). The reaction mixture was stirred at 37° C. and monitored by LC-MS. Upon completion, the reaction was quenched by the addition of MeCN/H₂O/AcOH (47.5:47.5:5) and further purified by HPLC.

F: ACM Protecting Group Removal.

To a solution of the purified product in 0.2 ml of degassed solvent HOAc: H₂O (3:1) was added AgOAc in one portion. The reaction mixture was stirred at rt and monitored by LC-MS. Upon completion, the reaction was quenched by the addition of 1 M of DTT in H₂O/AcOH (1:1), the resulting cloudy mixture was stirred for 20 min. Mixture was centrifuged and the supernatant was carefully taken out and lyophilized.

### Example 1

### Preparation GM-CSF Aglycone

### Side-Chain Protected Peptide S1

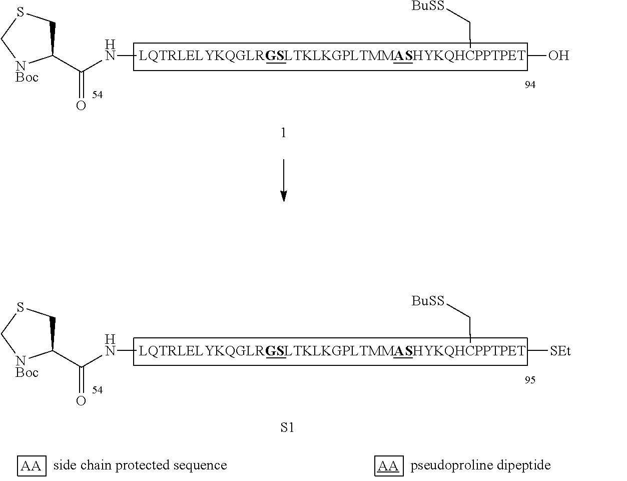

Fully protected peptide 1 was prepared according to General Procedure A for SPPS on a 0.1 mmol scale using Fmoc-Thr(OtBu)-NovaSyn® TGT resin, pseudoproline dipeptides Fmoc-Gly-Ser(ψ^{Me,Me}Pro)-OH, Fmoc-Ala-Ser(ψ^{Me,Me}Pro)-OH, special peptides Fmoc-Cys(SStBu)-OH, Boc-Thz-OH, and other standard Fmoc amino acids with acid-labile side-chain protections from Novabiochem®. After cleavage using the CH₂Cl₂/TFE/AcOH protocol, the crude material was concentrated in vacuo to afford peptide s1 (500.0 mg, 66%) as a white solid.

Following the General Procedure B, the fully protected peptidyl acid 1 (100 mg, 1.0 equiv) and HCl.H-Ser-SEt (7.3 mg, 3.0 equiv) were dissolved in 0.5 mL of CHCl₃ and cooled to −10° C. HOOBt (6.47 mg, 3.0 equiv) and EDCI free base (6.5 μL, 3.0 equiv) were then added. The reaction mixture was stirred at room temperature for 4 h. The solvent was gently blown off by a nitrogen stream and the residue was lyophilized overnight. 115 mg of crude peptide was obtained as a light yellow solid.

### Unprotected Peptide 2

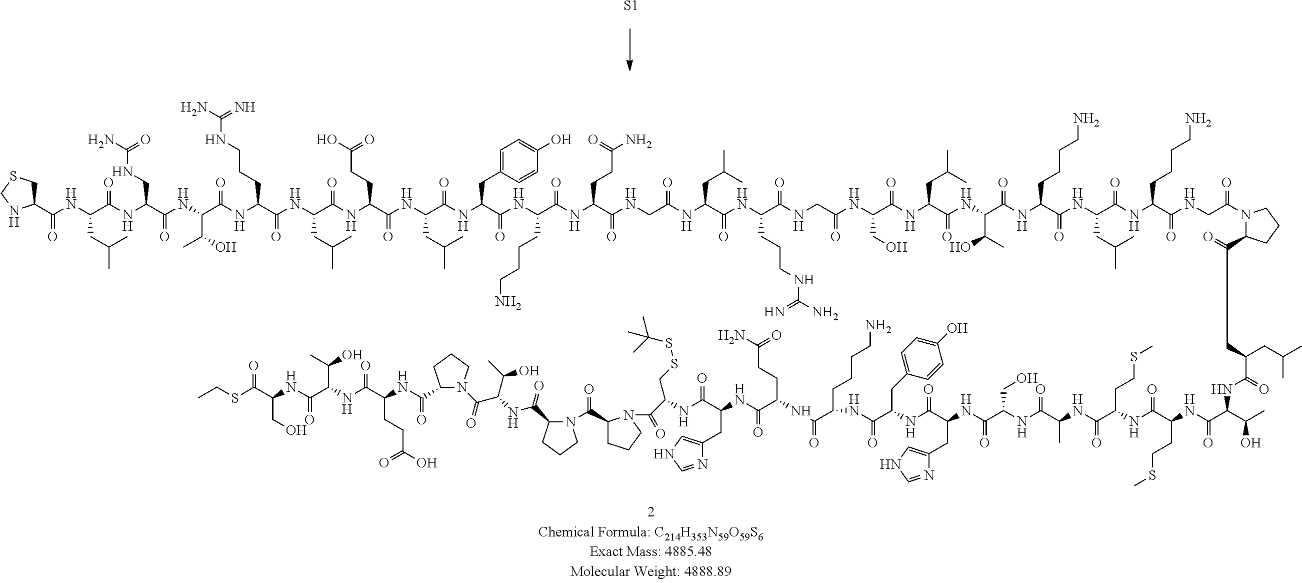

115 mg of S1 was placed in a 50 mL falcon test tube, 10 mL of TFA/TIS/H₂O/DMS (90:2.5:2.5:5 v/v) was added. The resulting solution was stirred at rt for 1 h, the liquid was blown off with nitrogen, and the oily residue was triturated with diethyl ether, and further purified by RP-HPLC (linear gradient 28-38% solvent B over 30 min, Microsorb 300-5 C4 column, 16 mL/min, 230 nm). Product eluted at 18-22 min. The fractions were collected, and concentrated via lyophilization to afford 30.0 mg glycopeptide 2 (46%) as a white solid. LC-MS and ESI-MS analysis of peptide 2: Calcd for C₂₁₄H₃₅₃N₅₉O₅₉S₆: 4888.89 Da (average isotopes), [M+3H]³⁺ m/z=1830.63, [M+4H]⁴⁺ m/z=1223.47, [M+5H]⁵⁺ m/z=978.78; observed: [M+3H]³⁺ m/z=1830.20, [M+4H]⁴⁺ m/z=1222.70, [M+5H]⁵⁺ m/z=978.50.

### Unprotected Peptide 3

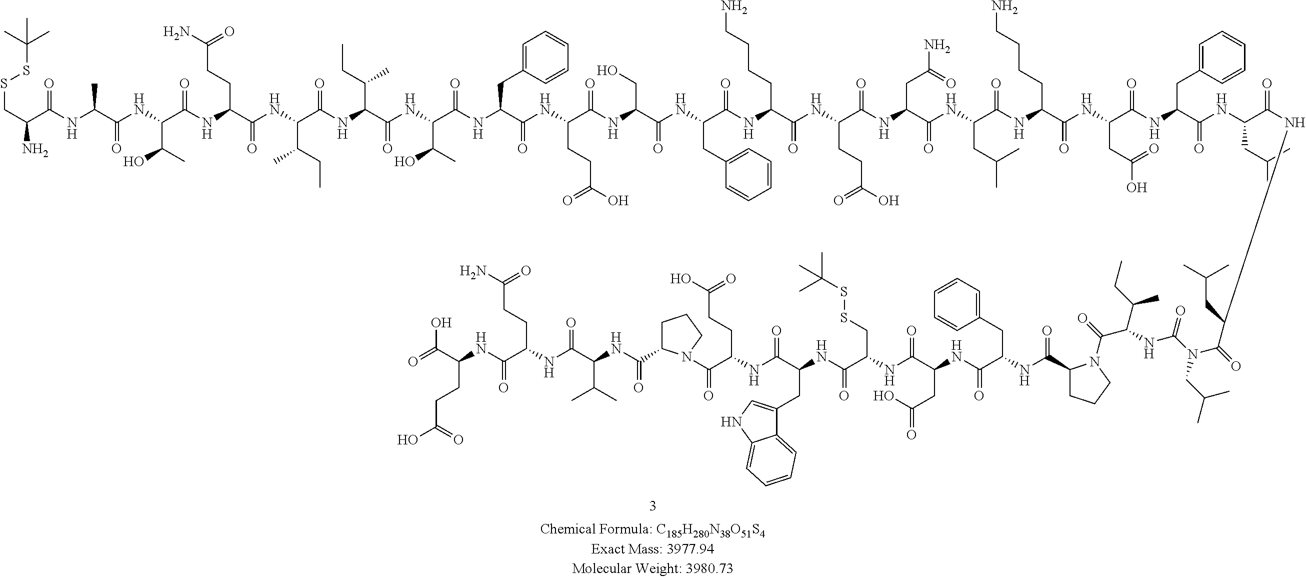

Following the general procedure for SPPS, peptide was synthesized on a 0.1 mmol scale by automated Applied Biosystems Pioneer continuous flow peptide synthesizer, with a mixture of 100:2:2 of DMF/Oxyma pure/DBU as the deblock reagent, and employing Fmoc-Glu-NovaSyn® TGT resin, pseudoproline dipeptides Fmoc-Glu-Ser(ψ^{Me,Me}Pro)-OH, peptides Fmoc-Asp(OMpe)-OH, Fmoc-Cys(SStBu)-OH, Boc-Cys(SStBu)-OH and other standard Fmoc amino acids with acid-labile side-chain protections from Novabiochem®. After cleavage using the CH₂Cl₂/TFE/AcOH protocol, the crude material was concentrated in vacuo to afford fully protected peptide as a white solid.

100 mg of fully protected peptide was placed in a 50 mL falcon test tube and 10 mL of TFA/TIS/H₂O/Phenol (88:5:5:2 v/v) was added. The resulting solution was stirred at rt for 2 h, the liquid was blown off with nitrogen, and the oily residue was triturated with diethyl ether, and further purified by RP-HPLC (linear gradient 40-70% solvent B over 30 min, Microsorb 300-5 C4 column, 16 mL/min, 230 nm). Product eluted at 15-17 min. The fractions were collected, and concentrated via lyophilization to afford peptide 3 (xx %) as a white solid. LC-MS and ESI-MS analysis of peptide 3: Calcd for C₁₈₅H₂₈₀N₃₈O₅₁S₄: 3980.73 Da (average isotopes), [M+2H]²⁺ m/z=1991.36, [M+3H]³⁺ m/z=1327.91, [M+4H]⁴⁺ m/z=996.18; observed: [M+2H]²⁺ m/z=1992.29, [M+3H]³⁺ m/z=1328.42, [M+4H]⁴⁺ m/z=977.07.

### Unprotected Peptide 6

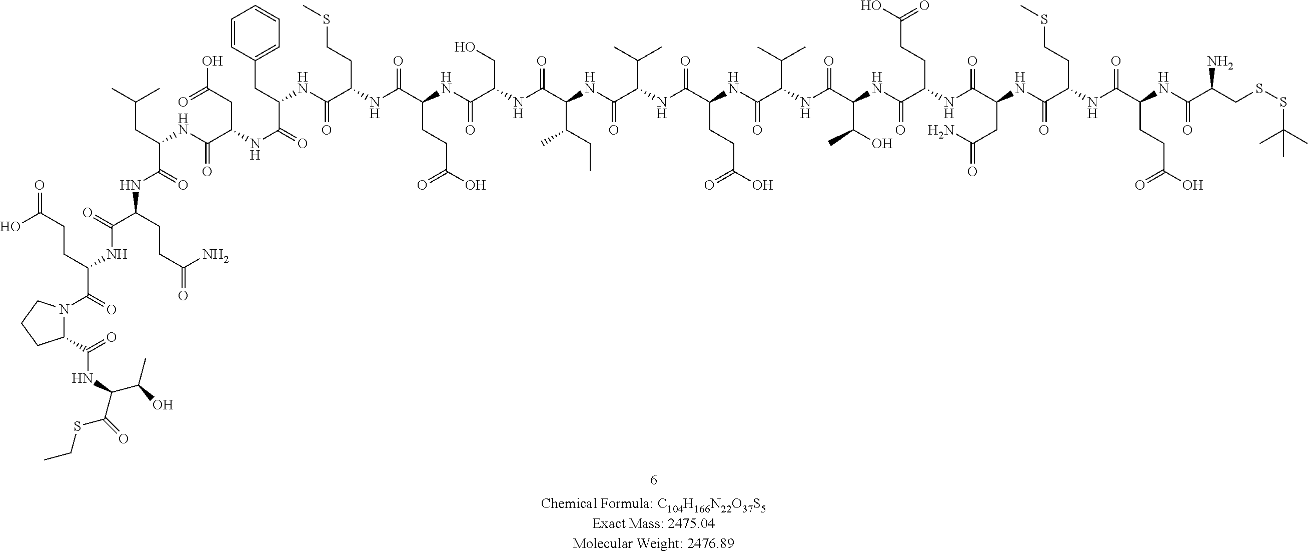 [SMILES: CCSC(=O)[C@@H](NC(=O)[C@@H]1CCCN1C(=O)[C@H](CCC(=O)O)NC(=O)[C@H](CCC(N)=O)NC(=O)[C@H](CC(C)C)NC(=O)[C@H](CC(=O)O)NC(=O)[C@H](Cc1ccccc1)NC(=O)[C@H](CCSC)NC(=O)[C@H](CCC(=O)O)NC(=O)[C@H](CO)NC(=O)[C@@H](NC(=O)[C@@H](NC(=O)[C@H](CCC(=O)O)NC(=O)[C@@H](NC(=O)[C@@H](NC(=O)[C@H](CCC(=O)O)NC(=O)[C@H](CC(N)=O)NC(=O)[C@H](CCSC)NC(=O)[C@H](CCC(=O)O)NC(=O)[C@@H](N)CSSC(C)(C)C)[C@H](C)O)C(C)C)C(C)C)[C@@H](C)CC)[C@@H](C)O]

Fully protected peptide GM-34-52 (i.e., Cys³⁴ to Pro⁵² of the GM-CSF sequence of SEQ ID NO:1, wherein Cys³⁴ is substituted for Ala³⁴) was prepared according to General Procedure A for SPPS on a 0.05 mmol scale using Fmoc-Pro-NovaSyn® TGT resin, pseudoproline dipeptides Fmoc-Ile-Ser(ψ^{Me,Me}Pro)-OH, Fmoc-Thr(OtBu)-Thr(ψ^{Me,Me}Pro)-OH, peptides Boc-Cys(SStBu)-OH and other standard Fmoc amino acids with acid-labile side-chain protections from Novabiochem®. After cleavage using the CH₂Cl₂/TFE/AcOH protocol, the crude material was concentrated in vacuo to afford fully protected peptide (120 mg, 80%) as a white solid.

Following the General Procedure B, the fully protected peptidyl acid GM-34-52 (i.e., Cys³⁴ to Pro⁵² of the GM-CSF sequence of SEQ ID NO:1, wherein Cys³⁴ is substituted for Ala³⁴) (72 mg, 1.0 equiv) and HCl.H-Thr(OtBu)-SEt (17.5 mg, 3.0 equiv) were dissolved in 0.3 mL of CHCl₃ and cooled to −10° C. HOOBt (11.2 mg, 3.0 equiv) and EDCI free base (11.3 μL, 3.0 equiv) were then added. The reaction mixture was stirred at room temperature for 4 h. The solvent was gently blown off by a nitrogen stream and the residue was lyophilized overnight. 85 mg of crude peptide was obtained as a light yellow solid.

40 mg of crude peptide was placed in a 15 mL falcon test tube, 5 mL of TFA/TIS/H₂O (95:2.5:2.5 v/v) was added. The resulting solution was stirred at rt for 1 h, the liquid was blown off with nitrogen, and the oily residue was triturated with diethyl ether, and further purified by RP-HPLC (linear gradient 40-70% solvent B over 30 min, Microsorb 300-5 C4 column, 16 mL/min, 230 nm). Product eluted at 9-11 min. The fractions were collected, and concentrated via lyophilization to afford 10.0 mg glycopeptide 6 (31.5%) as a white solid. LC-MS and ESI-MS analysis of peptide 6: Calcd for C₁₀₄H₁₆₆N₂₂O₃₇S₅: 2476.89 Da (average isotopes), [M+2H]²⁺ m/z=1239.44, [M+3H]³⁺ m/z=826.63; observed: [M+2H]²⁺ m/z=1239.75, [M+3H]³⁺ m/z=827.01.

### Unprotected Peptide 5

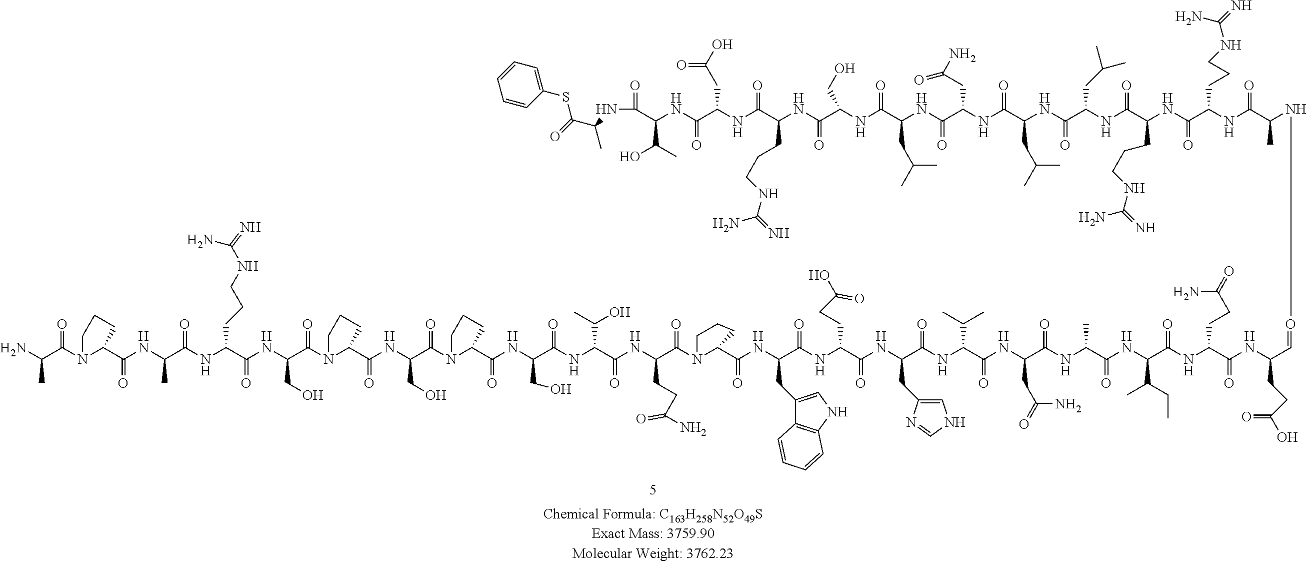

Fully protected peptide Gm-1-32 (i.e., Ala¹ to Thr³² of the GM-CSF sequence of SEQ ID NO:1) was prepared according to General Procedure A for SPPS on a 0.10 mmol scale using Fmoc-Thr-NovaSyn® TGT resin, pseudoproline dipeptide Fmoc-Leu-Ser(ψ^{Me,Me}Pro)-OH, peptides Boc-Ala-OH, Fmoc-Asp(OMpe) and other standard Fmoc amino acids with acid-labile side-chain protections from Novabiochem®. After cleavage using the CH₂Cl₂/TFE/AcOH protocol, the crude material was concentrated in vacuo to afford peptide (500 mg, 78%) as a white solid.

Following the General Procedure B, the fully protected peptidyl acid Gm-1-32 (i.e., Ala¹ to Thr³² of the GM-CSF sequence of SEQ ID NO:1) (109 mg, 1.0 equiv) and HCl.H-AlaSPh (10.7 mg, 3.0 equiv) were dissolved in 0.8 mL of CHCl₃ and cooled to −10° C. HOOBt (8.2 mg, 3.0 equiv) and EDCI free base (6.7 μL, 3.0 equiv) were then added. The reaction mixture was stirred at room temperature for 4 h. The solvent was gently blown off by a nitrogen stream and the residue was lyophilized overnight. 130 mg of crude peptide was obtained as a light yellow solid.

130 mg of crude peptide was placed in a 50 mL falcon test tube, 10 mL of TFA/TIS/H₂O (95:2.5:2.5 v/v) was added. The resulting solution was stirred at rt for 1 h, the liquid was blown off with nitrogen, and the oily residue was triturated with diethyl ether, and further purified by RP-HPLC (linear gradient 30-50% solvent B over 30 min, Microsorb 300-5 C4 column, 16 mL/min, 230 nm). Product eluted at 14-17 min. The fractions were collected, and concentrated via lyophilization to afford 30.0 mg glycopeptide 5 (39.0%) as a white solid. LC-MS and ESI-MS analysis of peptide 5: Calcd for C₁₆₃H₂₅₈N₅₂O₄₉S: 3762.23 Da (average isotopes), [M+3H]³⁺ m/z=1255.07, [M+4H]⁴⁺ m/z=941.55; observed: [M+3H]³⁺ m/z=1255.27, [M+4H]⁴⁺ m/z=941.86.

### GM-CSF Fragment 4

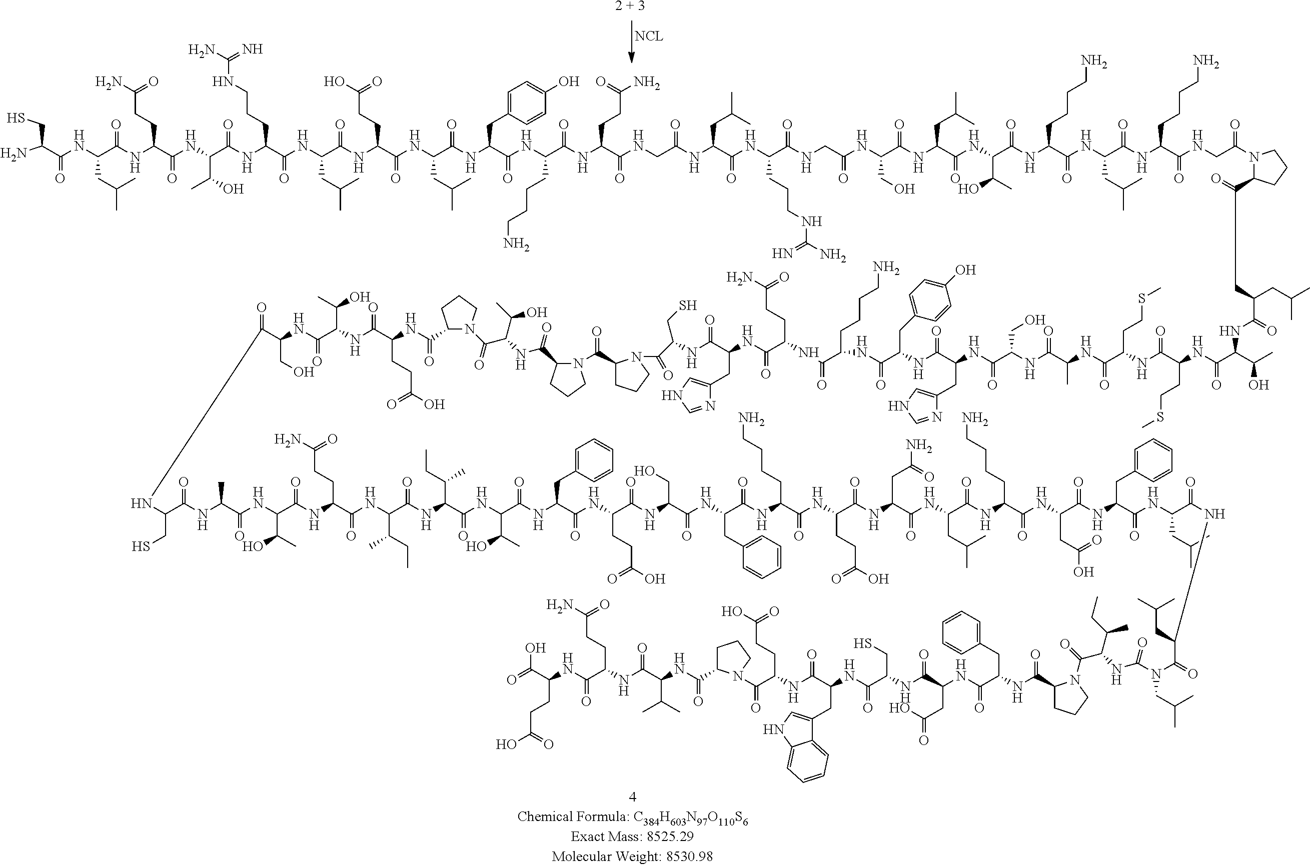

According to General Procedure D, peptides 2 (1.60 mg, 1.0 equiv) and 3 (1.40 mg, 1.10 equiv) were dissolved in 160 μL of NCL buffer under an argon atmosphere. The resulting mixture was stirred at room temperature and the reaction was monitored by LC-MS. After 15 h, to the reaction was added 4.5 mg of MeONH₂HCl in one portion. The resulting mixture was further stirred at rt for 3 h under Ar. The reaction was quenched with 3 mL of CH₃CN/H₂O/AcOH (30:65:5) and 100 μL of Bond-Breaker® TCEP solution, and then purified directly by RP-HPLC (linear gradient 35-55% solvent B over 30 min, Microsorb 300-5 C4 column, 16 mL/min, 230 nm). Product eluted at 15-17 min. The fractions were collected, and concentrated via lyophilization to afford 1.7 mg ligated peptide 4 (61%, two steps) as a white solid. LC-MS analysis of peptide 4: ESI-MS of peptide 4 calcd for C₃₈₄H₆₀₃N₉₇O₁₁₀S₆: 8530.98 Da [M+4H]⁴⁺ m/z=2133.74, [M+5H]⁵⁺ m/z=1707.19, [M+6H]⁶⁺ m/z=1422.83, [M+7H]⁷⁺ m/z=1219.71, [M+8H]⁸⁺ m/z=1067.37, [M+9H]⁹⁺ m/z=948.88. found: [M+4H]⁴⁺ m/z=2133.60, [M+5H]⁵⁺ m/z=1707.00, [M+6H]⁶⁺ m/z=1422.60, [M+7H]⁷⁺ m/z=1219.40, [M+8H]⁸⁺ m/z=1067.10, [M+9H]⁹⁺ m/z=948.60.

### GM-CSF Fragment 8

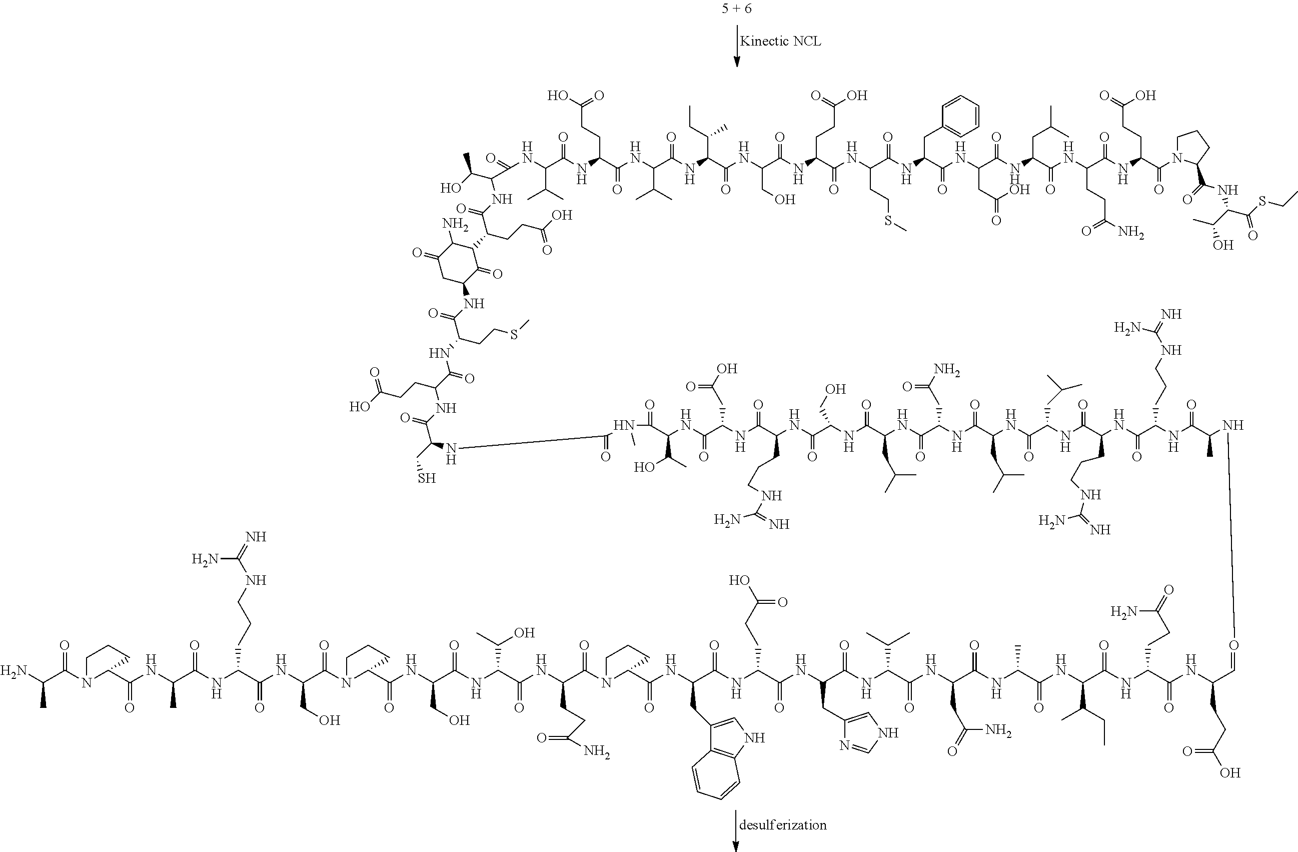

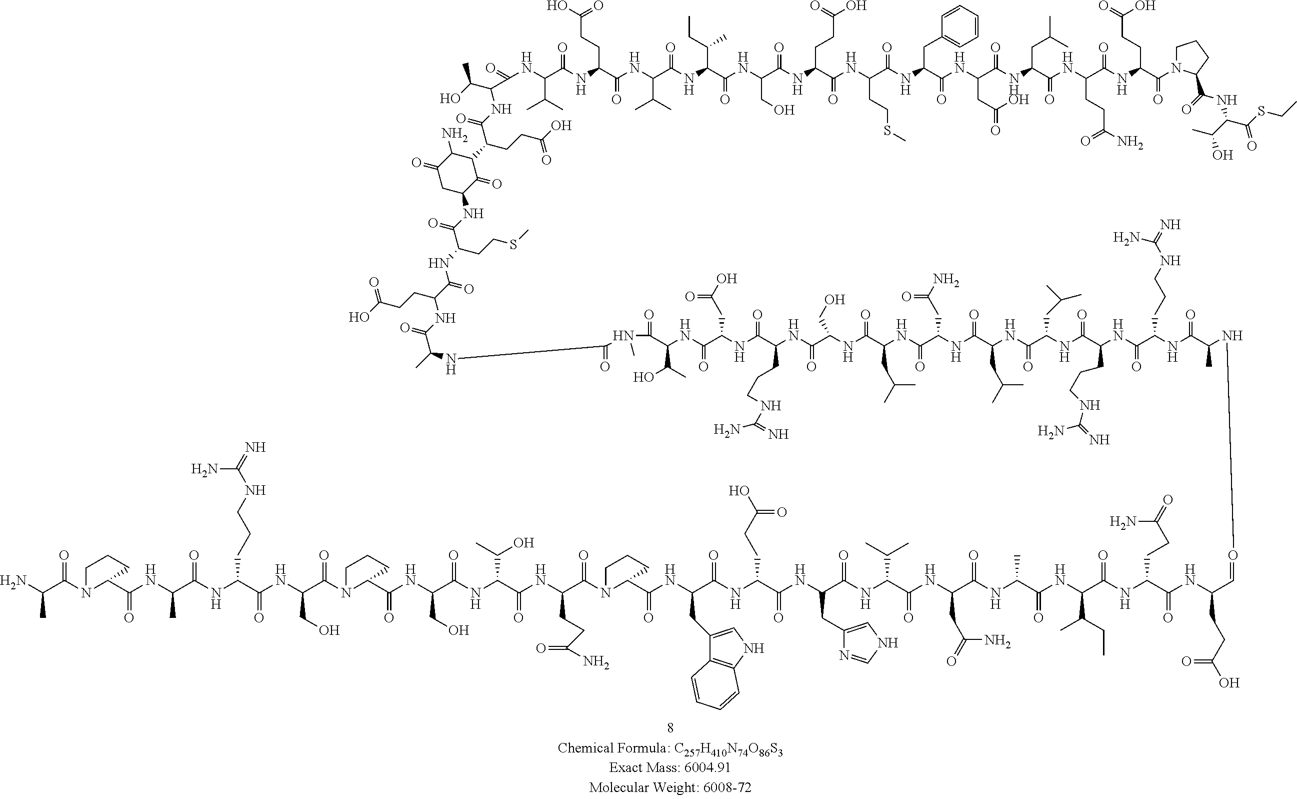

According to General Procedure C, peptides 5 (2.40 mg, 1.0 equiv) and 6 (1.60 mg, 1.0 equiv) were dissolved in 300 μL of Kinetic NCL buffer under an argon atmosphere. The resulting mixture 7 was stirred at room temperature and the reaction was monitored by LC-MS. After 15 h, according to General Procedure E, the crude reaction mixture 7 was added 0.1 ml of degassed buffer (6 M Gnd.HCl, 200 mM Na₂HPO₄) and followed by 0.2 ml of 0.5 M Bond-Breaker® TCEP solution (Pierce), 40 μL of 2-methyl-2-propanethiol and 70 μL of radical initiator VA-044 (0.1 M in H₂O). The reaction was stirred at 37° C. under an argon atmosphere for 3 h. The reaction was quenched with 3 mL of CH₃CN/H₂O/AcOH (30:65:5) and then purified directly by RP-HPLC (linear gradient 30-38% solvent B over 30 min, Microsorb 300-5 C4 column, 16 mL/min, 230 nm). Product eluted at 15-17 min. The fractions were collected, and concentrated via lyophilization to afford 1.7 mg ligated peptide 8 (49%, two steps) as a white solid. LC-MS analysis of peptide 8: ESI-MS of peptide 8 calcd for C₂₅₇H₄₁₀N₇₄O₈₆S₃: 6008.72 Da [M+3H]³⁺ m/z=2003.90, [M+4H]⁴⁺ m/z=1503.18 [M+5H]⁵⁺ m/z=1202.74, [M+6H]⁶⁺ m/z=1002.45. found: [M+3H]³⁺ m/z=2003.80, [M+4H]⁴⁺ m/z=1503.00 [M+5H]⁵⁺ m/z=1202.60, [M+6H]⁶⁺ m/z=1002.30.

### GM-CSF Primary Construct 9

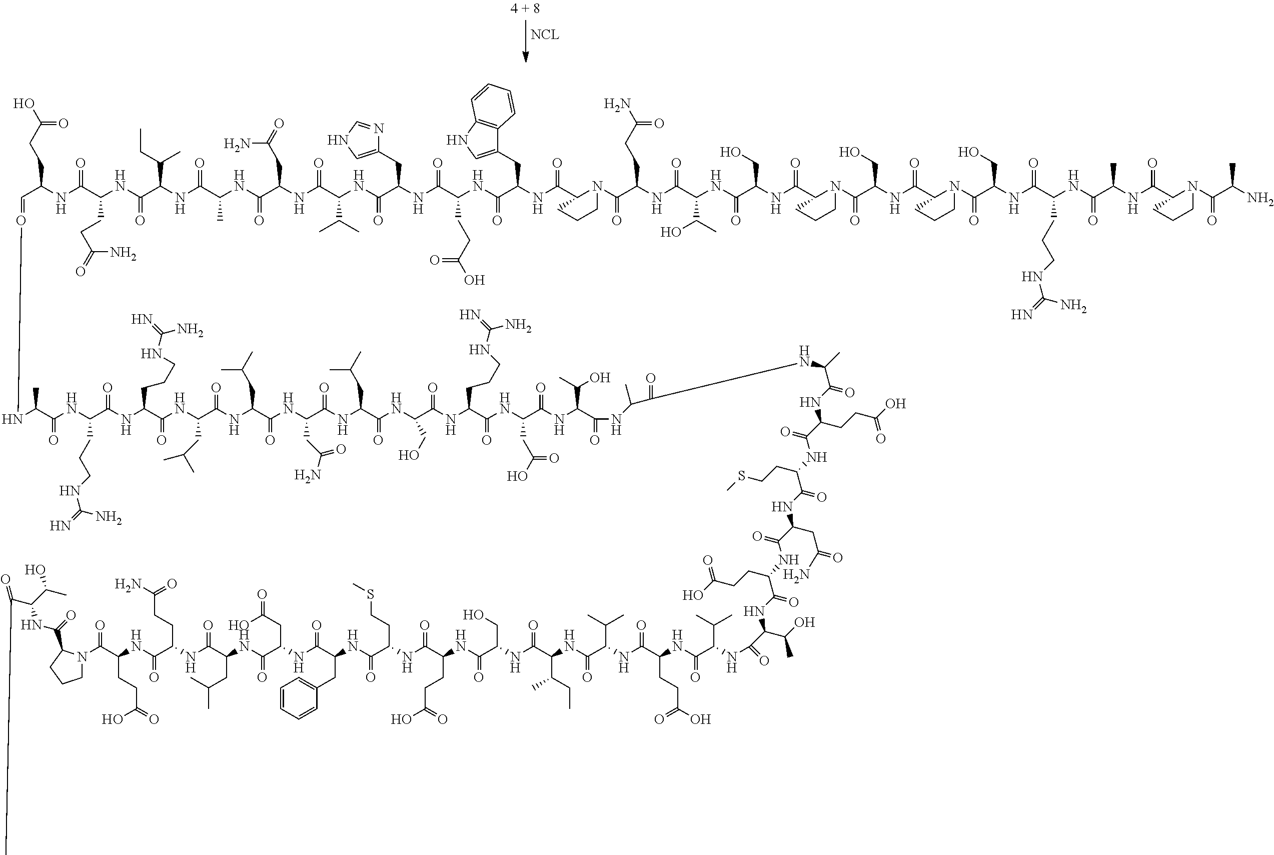

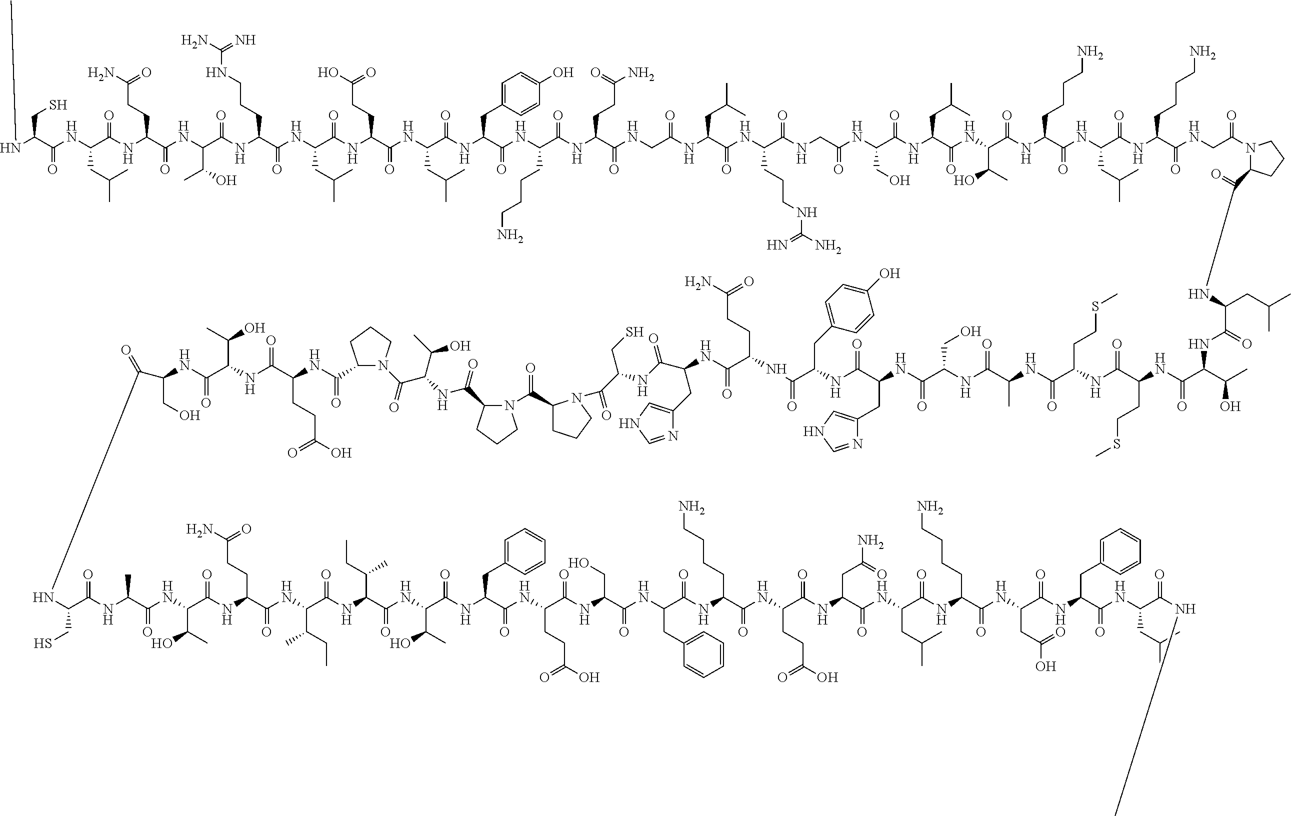

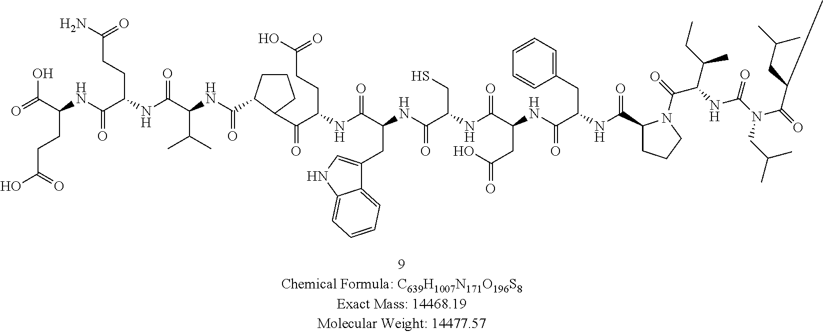

According to General Procedure D, peptides 8 (0.81 mg, 1.0 equiv) and 4 (1.14 mg, 1.20 equiv) were dissolved in 120 μL of NCL buffer under an argon atmosphere. The resulting mixture was stirred at room temperature and the reaction was monitored by LC-MS. After 15 h, the reaction was quenched with 3 mL of CH₃CN/H₂O/AcOH (30:65:5) and 100 μL of Bond-Breaker® TCEP solution, and then purified directly by RP-HPLC (linear gradient 40-60° A solvent B over 30 min, Microsorb 300-5 C4 column, 16 mL/min, 230 nm). Product eluted at 15-17 min. The fractions were collected, and concentrated via lyophilization to afford 1.00 mg GM-CSF primary sequence 9 (50%) as a white solid. LC-MS analysis of GM-CSF primary 9: ESI-MS of peptide 9 calcd for C₆₃₉H₁₀₀₇N₁₇₁O₁₉₆S₈: 14477.57 Da [M+9H]⁹⁺ m/z=1609.62, [M+10H]¹⁰⁺ m/z=1448.75, [M+11H]¹¹⁺ m/z=1317.14, [M+12H]¹²⁺ m/z=1207.46, [M+13H]¹³⁺ m/z=1114.66, [M+14H]¹⁴⁺ m/z=1035.11, [M+15H]¹⁵⁺ m/z=966.17, [M+16H]¹⁶⁺ m/z=905.85. found: [M+9H]⁹⁺ m/z=1609.60, [M+10H]¹⁰⁺ m/z=1448.60, [M+11H]¹¹⁺ m/z=1317.00, [M+12H]¹²⁺ m/z=1207.30, [M+13H]¹³⁺ m/z=1114.60, [M+14H]¹⁴⁺ m/z=1035.00, [M+15H]¹⁵⁺ m/z=966.00, [M+16H]¹⁶⁺ m/z=905.70.

### Example 2

### Synthesis of Glycosylated GM-CSF Fragment

### Unprotected Peptide 15:

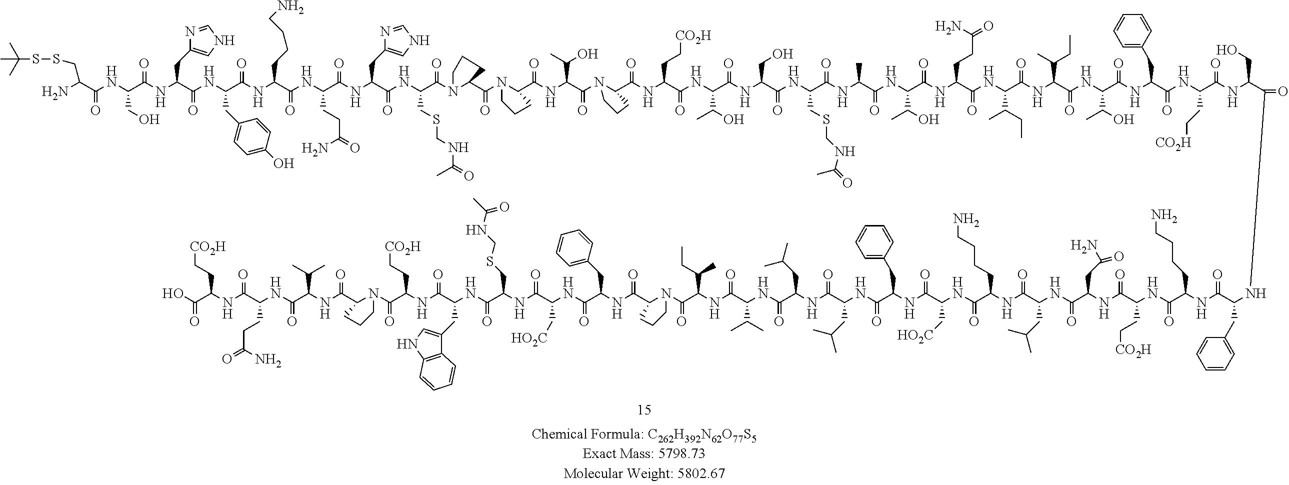

Following the general procedure for SPPS, peptide was synthesized on a 0.10 mmol scale by automated Applied Biosystems Pioneer continuous flow peptide synthesizer, with a mixture of 100:2:2 of DMF/Oxyma pure/DBU as the deblock reagent, employing Fmoc-Glu-NovaSyn® TGT resin, pseudoproline dipeptide Fmoc-Glu-Ser(ψ^{Me,Me}Pro)-OH, Fmoc-Ala-Thr(ψ^{Me,Me}Pro)-OH, peptides Fmoc-Cys(SStBu)-OH, Fmoc-Asp(OMpe) and other standard Fmoc amino acids with acid-labile side-chain protections from Novabiochem®. After cleavage using the CH₂Cl₂/TFE/AcOH protocol, the crude material was concentrated in vacuo to afford crude peptide (400 mg, 69%) as a white solid.

100 mg of crude peptide was placed in a 50 mL falcon test tube, 10 mL of TFA/TIS/H₂O/Phenol (88:5:5:2 v/v) was added. The resulting solution was stirred at rt for 2 h, the liquid was blown off with nitrogen, and the oily residue was triturated with diethyl ether, and further purified by RP-HPLC (linear gradient 37-50% solvent B over 30 min, Microsorb 300-5 C8 column, 16 mL/min, 230 nm). Product eluted at 15-17 min. The fractions were collected, and concentrated via lyophilization to afford glycopeptide 15 as a white solid. LC-MS analysis of GM-CSF primary 15: ESI-MS of peptide 15 calcd for C₂₆₂H₃₉₂N₆₂O₇₇S₅: 5802.67 Da [M+3H]³⁺ m/z=1935.22, [M+4H]⁴⁺ m/z=1451.67, [M+5H]⁵⁺ m/z=1161.53, [M+6H]⁶⁺ m/z=968.11, [M+7H]⁷⁺ m/z=829.95. found: [M+3H]³⁺ m/z=1935.86, [M+4H]⁴⁺ m/z=1452.01, [M+5H]⁵⁺ m/z=1161.86, [M+6H]⁶⁺ m/z=968.47, [M+7H]⁺ m/z=830.23.

### Fully Protected Glycopeptide 13

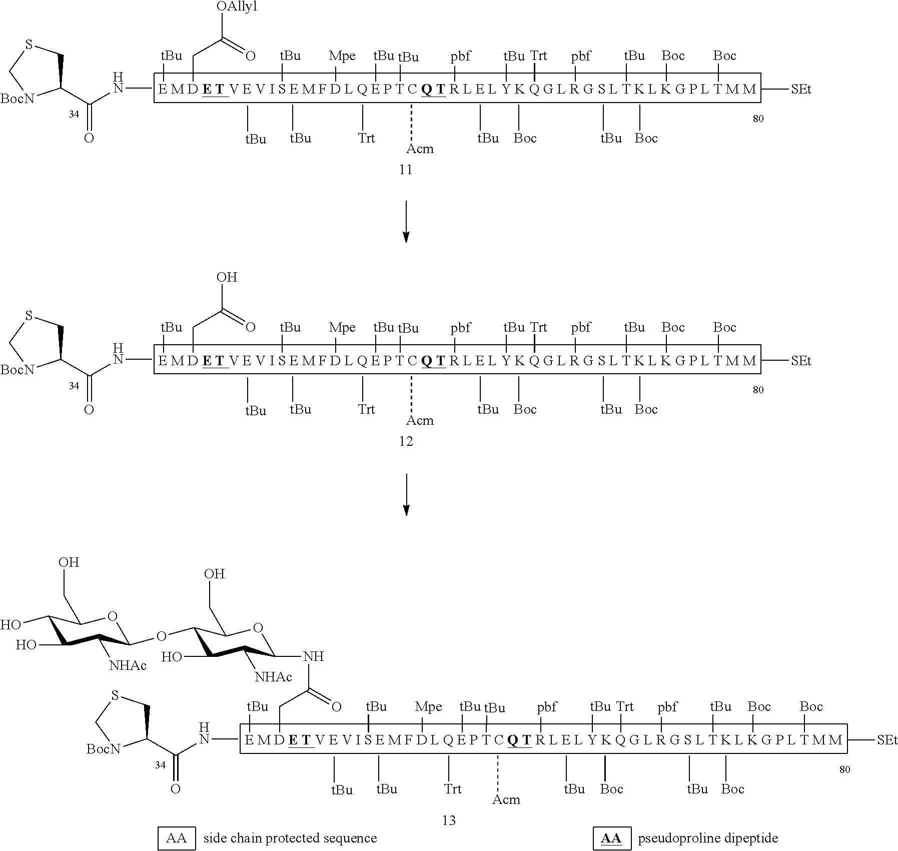

Following the general procedure for SPPS, peptide was synthesized on a 0.10 mmol scale by automated Applied Biosystems Pioneer continuous flow peptide synthesizer, with a mixture of 100:2:2 of DMF/piperidine/DBU as the deblock reagent, employing Fmoc-Met-NovaSyn®TGT resin, pseudoproline dipeptide Fmoc-Gln-Thr(ψ^{Me,Me}Pro)-OH, Fmoc-Glu-Thr(ψ^{Me,Me}Pro)-OH, peptides Fmoc-Asp(OAllyl)-OH, Fmoc-Asp(OMpe), Boc-Thz-OH and other standard Fmoc amino acids with acid-labile side-chain protections from Novabiochem®. After cleavage using the CH₂Cl₂/TFE/AcOH protocol, the crude material was concentrated in vacuo to afford peptide GM-34-79 (i.e., Cys³⁴-Met⁷⁹ of the GM-CSF sequence of SEQ ID NO:1, wherein Cys³⁴ is substituted for Ala³⁴) (400 mg, 50%) as a white solid.

Following the General Procedure B, the fully protected peptidyl acid GM-34-79 (i.e., Cys³⁴-Met⁷⁹ of the GM-CSF sequence of SEQ ID NO:1, wherein Cys³⁴ is substituted for Ala³⁴) (109 mg, 1.0 equiv) and HCl.H-Met-SEt (10.7 mg, 3.0 equiv) were dissolved in 0.8 mL of CHCl₃ and cooled to −10° C. HOOBt (8.2 mg, 3.0 equiv) and EDCI free base (6.7 μL, 3.0 equiv) were then added. The reaction mixture was stirred at room temperature for 4 h. The solvent was gently blown off by a nitrogen stream and the residue was lyophilized overnight. 110 mg of crude peptide 11 was obtained as a light yellow solid. To a solution of crude peptide 11 (110 mg, 1 equiv) and Pd(PPh₃)₄ (3.2 mg, 0.20 equiv) in CH₂Cl₂ (6.0 mL) was added PhSiH₃ (90 μL, 20 equiv). The light yellow, clear solution was stirred at rt for 20 minutes. The reaction was concentrated under a stream of nitrogen and the residue was passed through short silica gel column (5%-10% MeOH/CH₂Cl₂), the fraction was concentrated and lyophilized to give a white solid 12 (50 mg, 46%).

To a mixture of peptide 14 (35.5 mg, 1.0 equiv), chitobiose (5.4 mg, 3 equiv) and HATU (5.0 mg, 3 equiv) was added DMSO (500 μL) and DIPEA (2.32 μL, 3 equiv). The reaction mixture was stirred at room temperature for 2 h. The crude mixture was lyophilized to give 40 mg a yellow solid 13.

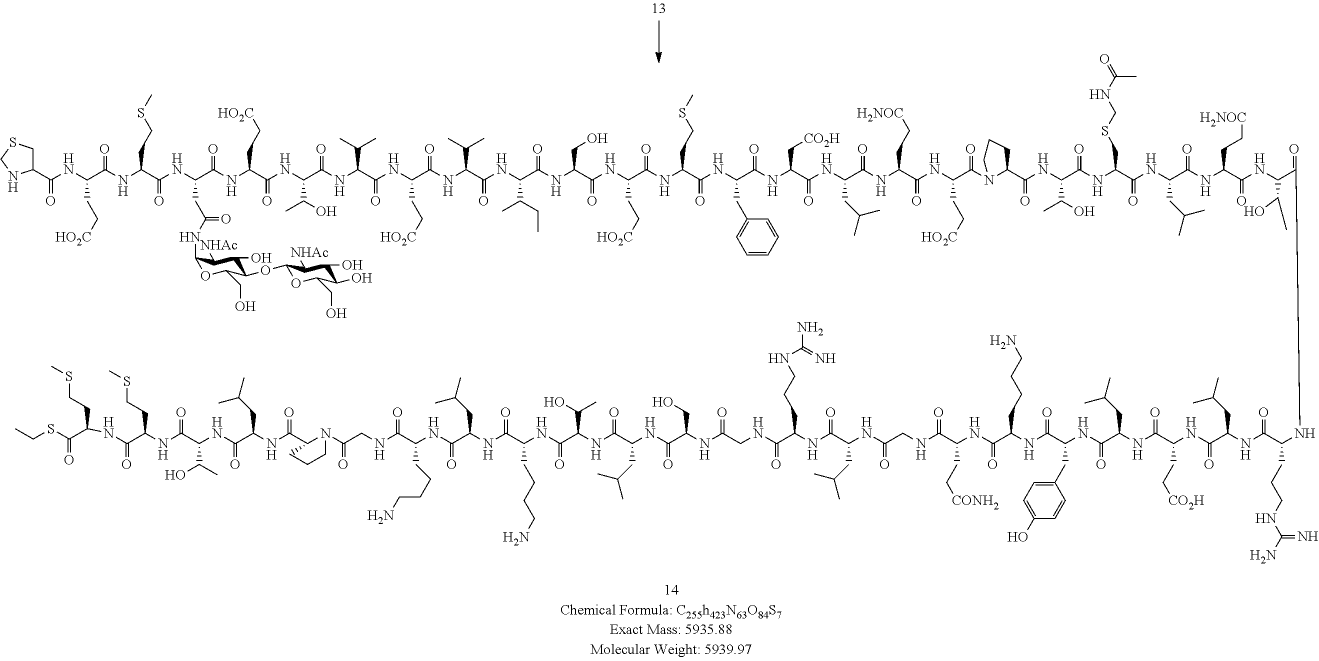

40 mg of 13 was placed in a 50 mL falcon test tube, 10 mL of TFA/TIS/H₂O/DMS (90:2.5:2.5:5 v/v) was added. The resulting solution was stirred at rt for 25 min, the liquid was blown off with nitrogen, and the oily residue was triturated with diethyl ether, and further purified by RP-HPLC (linear gradient 37-50% solvent B over 30 min, Microsorb 300-5 C8 column, 16 mL/min, 230 nm). Product eluted at 15-17 min. The fractions were collected, and concentrated via lyophilization to afford 5.5 mg glycopeptide 14 (21%) as a white solid. LC-MS analysis of GM-CSF primary 14: ESI-MS of peptide 14 calcd for C₂₅₅H₄₂₃N₆₃O₈₄S₇: 5939.97 Da [M+3H]³⁺ m/z=1980.99, [M+4H]⁴⁺ m/z=1485.99, [M+5H]⁵⁺ m/z=1188.99, [M+6H]⁶⁺ m/z=990.99, [M+7H]⁺ m/z=849.56. found: [M+3H]³⁺ m/z=1980.50, [M+4H]⁴⁺ m/z=1485.70, [M+5H]⁵⁺ m/z=1188.60, [M+6H]⁶⁺ m/z=990.60, [M+7H]⁺ m/z=849.50.

### Example 3

### Synthesis of GM-CSF Analogue 21

### Glycopeptide 17

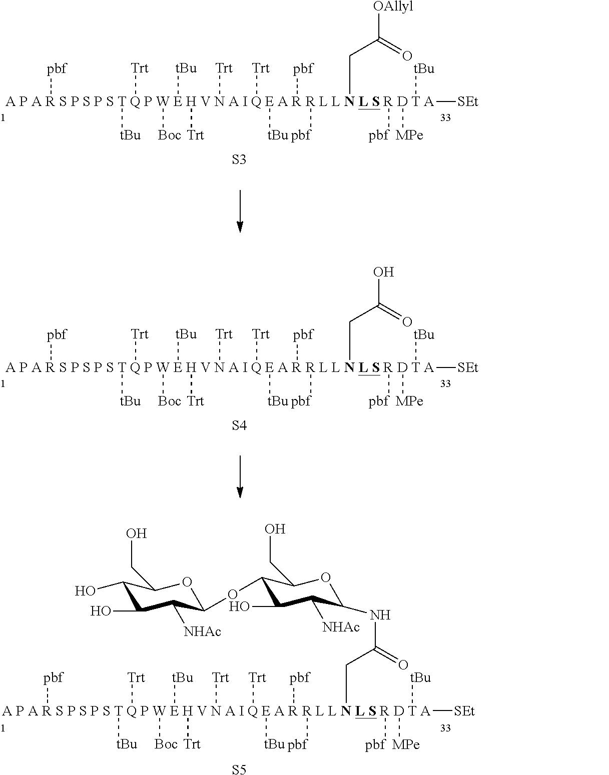

Following the general procedure for SPPS, peptide was synthesized on a 0.10 mmol scale by automated Applied Biosystems Pioneer continuous flow peptide synthesizer, with a mixture of 100:2:2 of DMF/piperidine/DBU as the deblock reagent, employing Fmoc-Thr(OtBu)-NovaSyn® TGT resin, pseudoproline dipeptide Fmoc-Leu-Ser(ψ^{Me,Me}Pro)-OH, peptides Fmoc-Asp(OAllyl)-OH, Fmoc-Asp(OMpe), Boc-Ala-OH and other standard Fmoc amino acids with acid-labile side-chain protections from Novabiochem®. After cleavage using the CH₂Cl₂/TFE/AcOH protocol, the crude material was concentrated in vacuo to afford peptide GM-1-32 (i.e., Ala¹ to Thr³² of the GM-CSF sequence of SEQ ID NO:1) (500 mg, 78%) as a white solid.

Following the General Procedure B, the fully protected peptidyl acid GM-1-32 (i.e., Ala¹ to Thr³² of the GM-CSF sequence of SEQ ID NO:1) (100 mg, 1.0 equiv) and HCl.H-AlaSEt (8.0 mg, 3.0 equiv) were dissolved in 0.3 mL of CHCl₃ and cooled to −10° C. HOOBt (7.6 mg, 3.0 equiv) and EDCI free base (6.1 μL, 3.0 equiv) were then added. The reaction mixture was stirred at room temperature for 3 h. The solvent was gently blown off by a nitrogen stream and the residue was lyophilized overnight. 105 mg of crude peptide S3 was obtained as a light yellow solid. To a solution of crude peptide S3 (105 mg, 1 equiv) and Pd(PPh₃)₄ (3.7 mg, 0.20 equiv) in CH₂Cl₂ (2.0 mL) was added PhSiH₃ (40 μL, 20 equiv). The light yellow, clear solution was stirred at rt for 20 minutes. The reaction was concentrated under a stream of nitrogen and the residue was passed through LH-20 gel column (5% MeOH/CH₂Cl₂), the faction was concentrated and lyophilized to give a pale yellow solid S4 (60 mg, 57%).

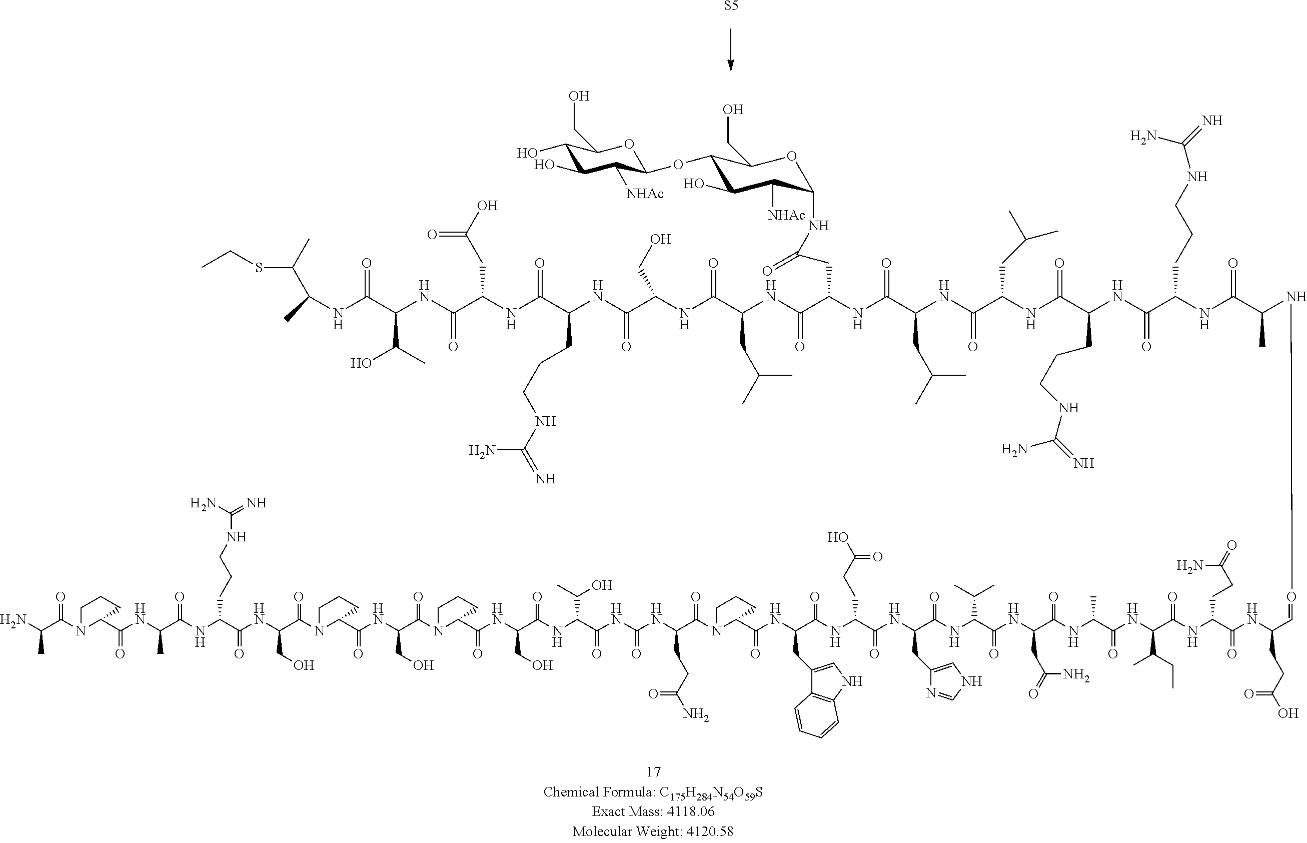

To a mixture of peptide S4 (50 mg, 1.0 equiv), chitobiose (9.4 mg, 3 equiv) and HATU (9.0 mg, 3 equiv) was added DMSO (300 μL) and DIPEA (4.1 μL, 3 equiv). The reaction mixture was stirred at room temperature for 3 h. The crude mixture was lyophilized to give a yellow solid S5.

Crude S5 was placed in a 15 mL falcon test tube, 5 mL of TFA/TIS/H₂O (95:2.5:2.5 v/v) was added. The resulting solution was stirred at rt for 2 hour, the liquid was blown off with nitrogen, and the oily residue was triturated with diethyl ether, and further purified by RP-HPLC (linear gradient 34-42% solvent B over 30 min, Microsorb 300-5 C4 column, 16 mL/min, 230 nm). Product eluted at 13-17 min. The fractions were collected, and concentrated via lyophilization to afford 10.0 mg glycopeptide 17 (50%) as a white solid. LC-MS analysis of GM-CSF primary 17: ESI-MS of peptide 17 calcd for C₁₇₅H₂₈₄N₅₄O₅₉S: 4120.58 Da [M+3H]³⁺ m/z=1374.52, [M+4H]⁴⁺ m/z=1031.14, [M+51-1]⁵⁺ m/z=825.11. found: [M+31-1]³⁺ m/z=1374.30, [M+4H]⁴⁺ m/z=1031.70, [M+51-1]⁵⁺ m/z=825.80.

### Glycopeptide 16

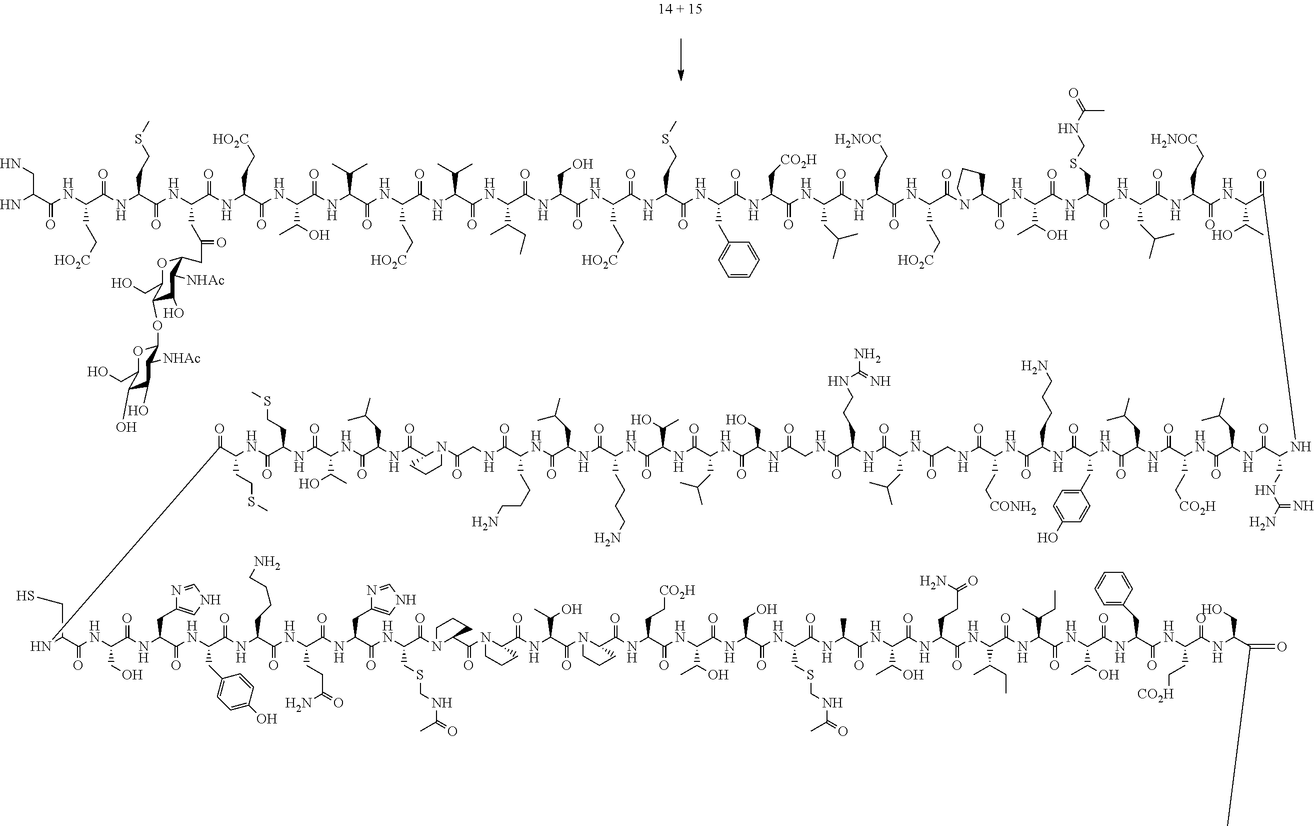

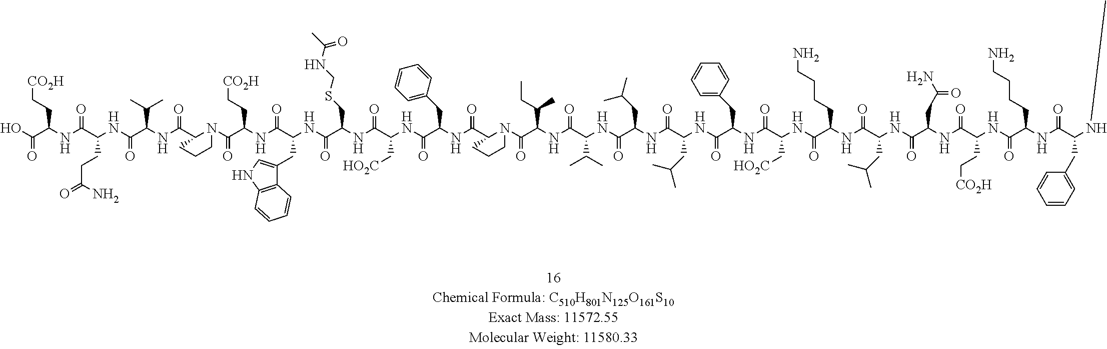 [SMILES: CC[C@@H](C)[C@@H](NC(=O)[C@H](NC(=O)[C@@H](CC(C)C)NC(=O)[C@@H](CC(C)C)NC(=O)[C@@H](Cc1ccccc1)NC(=O)[C@@H](CC(=O)O)NC(=O)[C@@H](CCCCN)NC(=O)[C@@H](CC(C)C)NC(=O)[C@@H](CC(N)=O)NC(=O)[C@@H](CCC(=O)O)NC(=O)[C@@H](CCCCN)NC(=O)[C@@H](Cc1ccccc1)NC)C(C)C)C(=O)N1CCC[C@@H]1C(=O)N[C@H](Cc1ccccc1)C(=O)N[C@H](CC(=O)O)C(=O)N[C@H](CSCNC(C)=O)C(=O)N[C@H](Cc1c[nH]c2ccccc12)C(=O)N[C@H](CCC(=O)O)C(=O)N1CCC[C@@H]1C(=O)N[C@@H](C(=O)N[C@H](CCC(N)=O)C(=O)N[C@H](CCC(=O)O)C(=O)O)C(C)C]

According to General Procedure D, glycopeptides 14 (2.50 mg, 1.00 equiv) and 15 (2.55 mg, 1.05 equiv) were dissolved in 200 μL of NCL buffer under an argon atmosphere. The resulting mixture was stirred at room temperature and the reaction was monitored by LC-MS. After 15 h, to the reaction was added 8 mg of MeONH₂HCl in one portion. The resulting mixture was further stirred at rt for three and a half hours under Ar. The reaction was quenched with 3 mL of CH₃CN/H₂O/AcOH (30:65:5) and 100 μL of Bond-Breaker® TCEP solution, and then purified directly by RP-HPLC (linear gradient 35-60% solvent B over 30 min, Microsorb 300-5 C4 column, 16 mL/min, 230 nm). Product eluted at 16-20 min. The fractions were collected, and concentrated via lyophilization to afford 3.0 mg ligated peptide 16 (61%, two steps) as a white solid. ESI-MS analysis of glycopeptide 16: Calcd for C₅₁₀H₈₀₁N₁₂₅O₁₆₁S₁₀: 11580.33 Da(average isotopes), [M+5H]⁵⁺ m/z=2317.06, [M+6H]⁶⁺ m/z=1931.05, [M+7H]⁷⁺ m/z=1655.33, [M+8H]⁸⁺ m/z=1448.54, [M+9H]⁹⁺ m/z=1287.70, [M+10H]¹⁰⁺ m/z=1159.03, [M+11H]¹¹⁺ m/z=1053.76; observed: [M+5H]⁵⁺ m/z=2317.70, [M+6H]⁶⁺ m/z=1931.00, [M+7H]⁺ m/z=1655.20, [M+8H]⁸⁺ m/z=1448.50, [M+9H]⁹⁺ m/z=1287.30, [M+10H]¹⁰⁺ m/z=1159.20, [M+11H]¹¹⁺ m/z=1053.70.

### GM-CSF Analogue Full Sequence S6

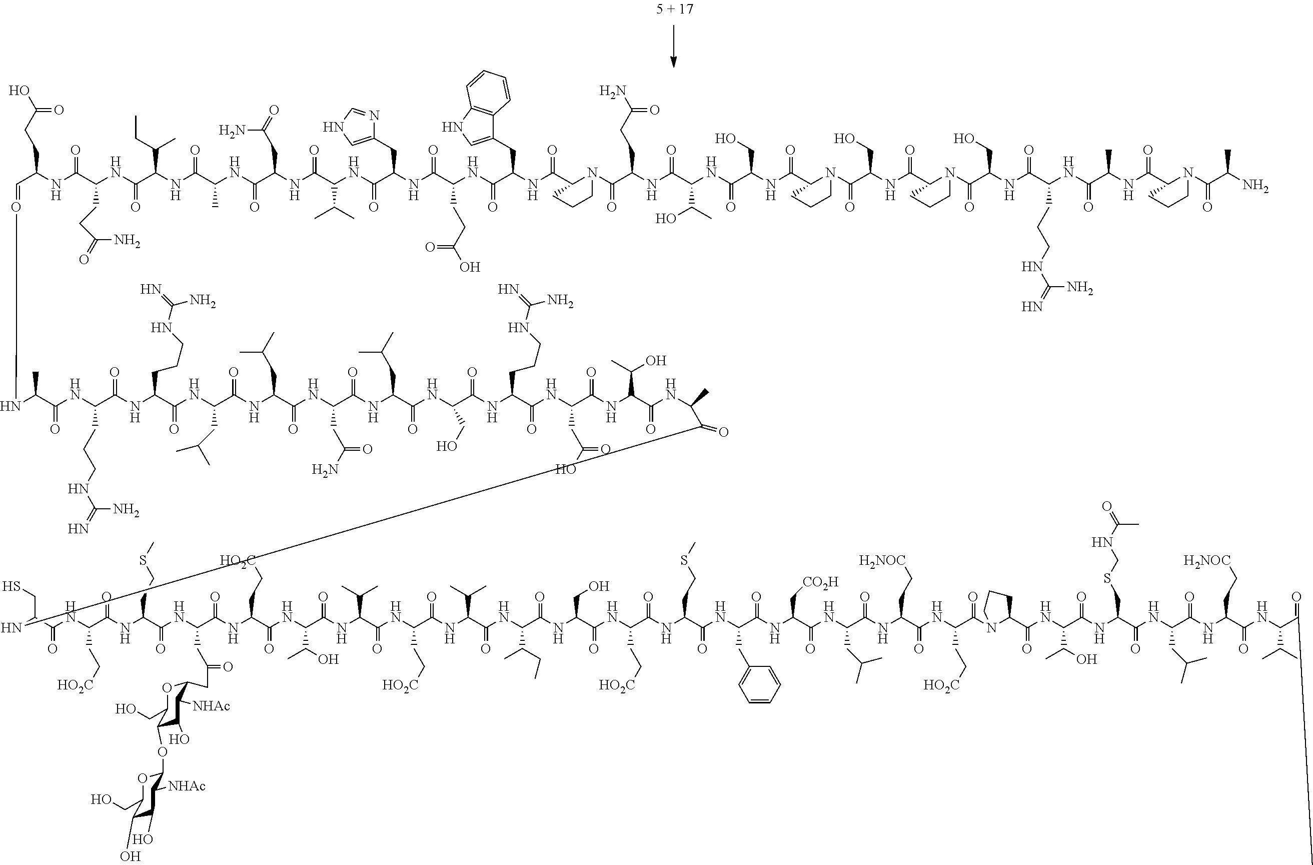

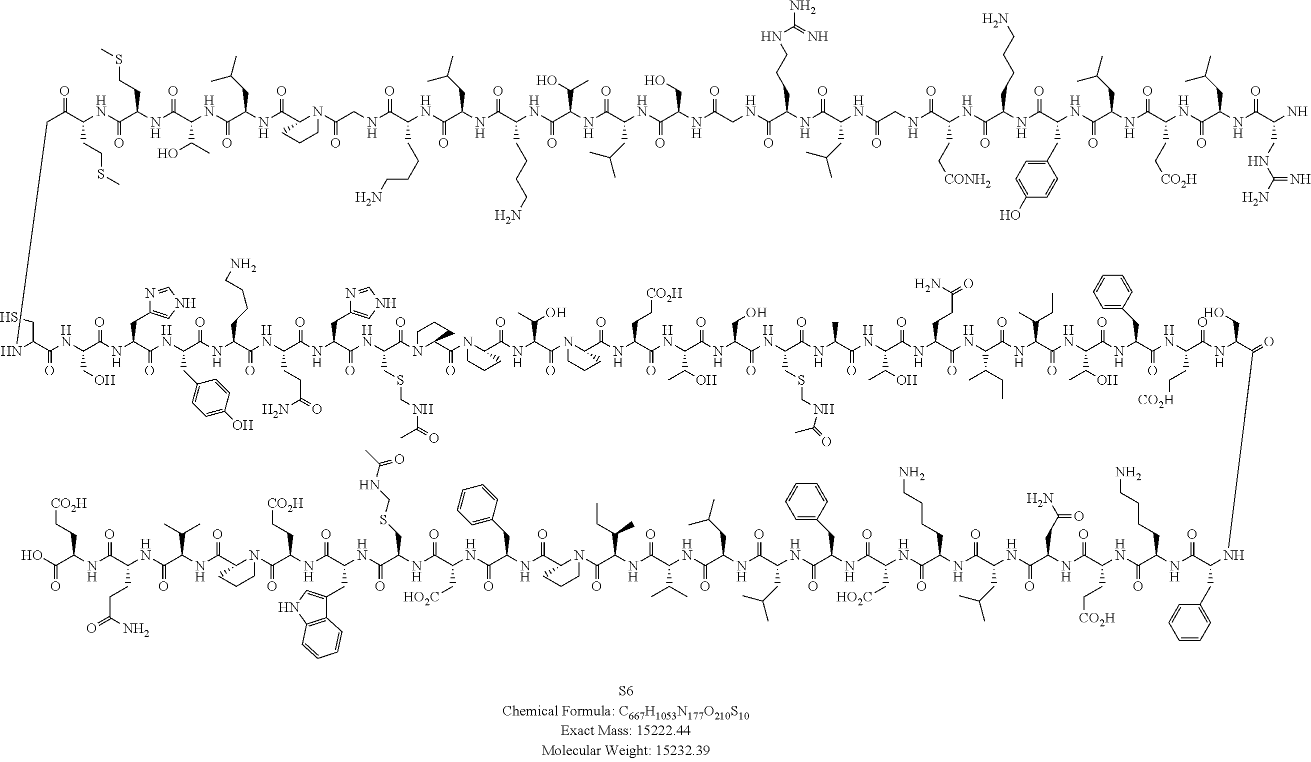

According to General Procedure D, glycopeptides 17 (6.0 mg, 1.00 equiv) and 5 (2.43 mg, 1.25 equiv) were dissolved in 400 μL of NCL buffer under an argon atmosphere. The resulting mixture was stirred at room temperature and the reaction was monitored by LC-MS. After 15 h, the reaction was quenched with 3 mL of CH₃CN/H₂O/AcOH (30:65:5) and 100 μL of Bond-Breaker® TCEP solution, and then purified directly by RP-HPLC (linear gradient 40-60% solvent B over 30 min, Microsorb 300-5 C4 column, 16 mL/min, 230 nm). Product eluted at 15-19 min. The fractions were collected, and concentrated via lyophilization to afford 4.90 mg ligated peptide S6 (63%) as a white solid. ESI-MS analysis of glycopeptide S6: Calcd for C₆₆₇H₁₀₅₃N₁₇₇O₂₁₀S₁₀: 15232.39 Da (average isotopes), [M+8H]⁸⁺ m/z=1905.04, [M+9H]⁹⁺ m/z=1693.49, [M+10H]¹⁹⁺ m/z=1524.24, [M+11H]¹¹⁺ m/z=1385.76, [M+12H]¹²⁺ m/z=1270.36, [M+13H]¹³⁺ m/z=1172.72, [M+14H]¹⁴⁺ m/z=1089.02, [M+15H]¹⁵⁺ m/z=1016.49, [M+16H]¹⁶⁺ m/z=953.02; observed: [M+8H]⁸⁺ m/z=1904.80, [M+9H]⁹⁺ m/z=1693.50, [M+10H]¹⁰⁺ m/z=1524.10, [M+11H]¹¹⁺ m/z=1385.60, [M+12H]¹²⁺ m/z=1270.20, [M+13H]¹³⁺ m/z=1172.60, [M+14H]¹⁴⁺ m/z=1088.80, [M+15H]¹⁵⁺ m/z=1016.30, [M+16H]¹⁶⁺ m/z=952.80.

### GM-CSF Analogue ACM S7

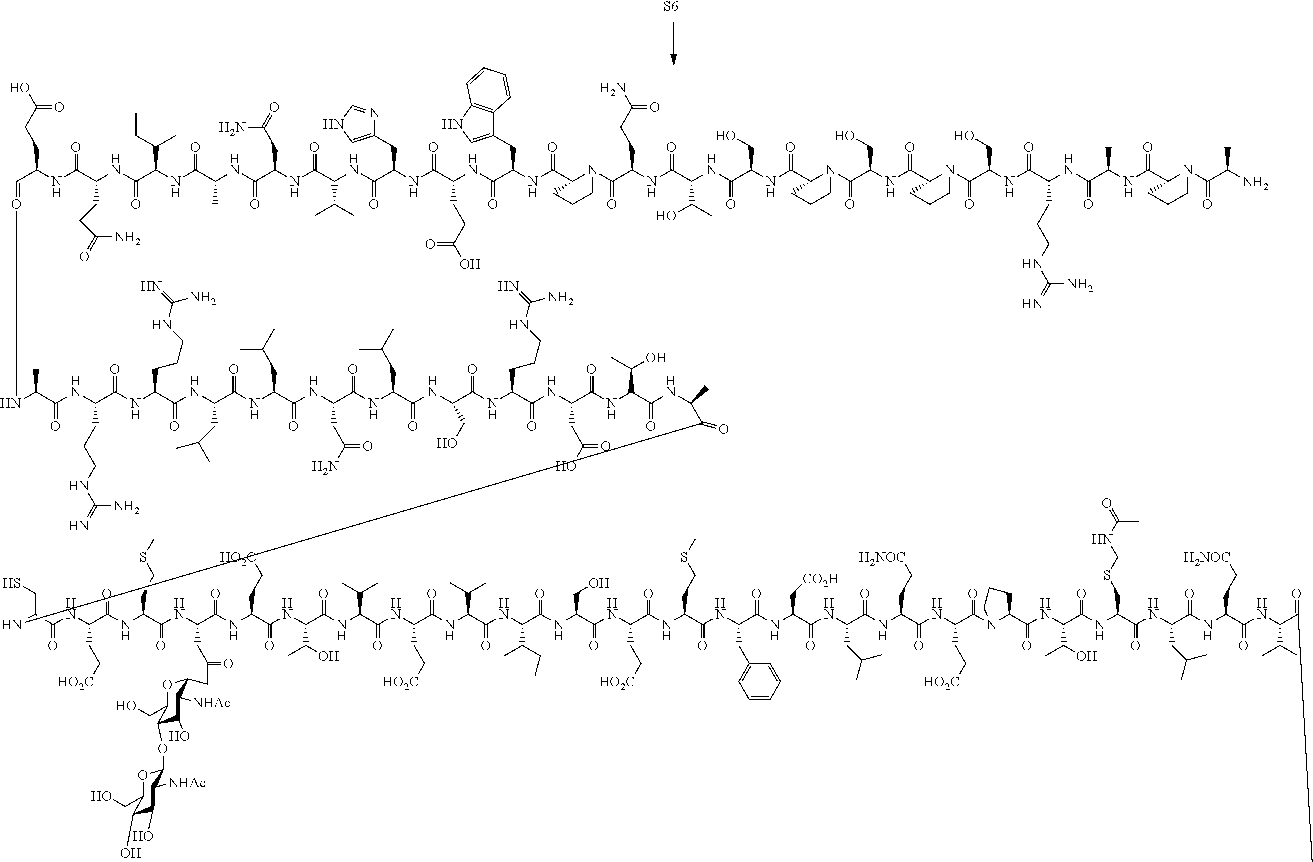

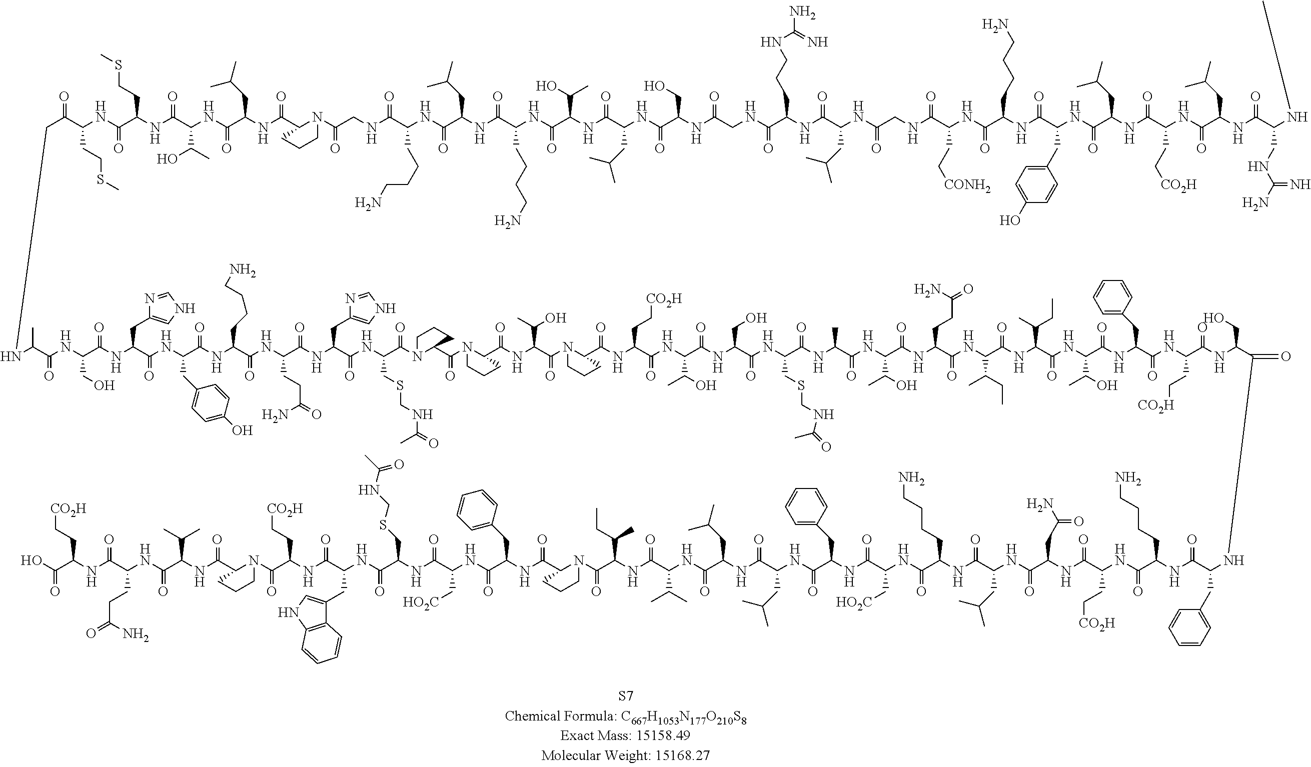

According to General Procedure E, glycopeptides S6 (2.5 mg, 1.0 equiv) in degassed buffer (6 M Gnd.HCl, 200 mM Na₂HPO₄) was added 0.2 ml of 0.5 M Bond-Breaker® TCEP solution, 0.04 ml of 2-methyl-2-propanethiol and 0.075 ml of radical initiator VA-044 (0.1 M in H₂O). The reaction mixture was stirred at 37° C. and monitored by LC-MS. Upon completion at 5 h, the reaction was quenched by the addition of MeCN/H₂O/AcOH (47.5:47.5:5) and purified by RP-HPLC (linear gradient 40-60% solvent B over 30 min, Microsorb 300-5 C4 column, 16 mL/min, 230 nm). Product eluted at 15-19 min. The fractions were collected, and concentrated via lyophilization to afford 2.00 mg glycopeptide S7 (80%) as a white solid. ESI-MS analysis of glycopeptide S7: Calcd for C₆₆₇H₁₀₅₃N₁₇₇O₂₁₀S₈: 15168.27 Da (average isotopes), [M+8H]⁸⁺ m/z=1897.03, [M+9H]⁹⁺ m/z=1686.36, [M+10H]¹⁰⁺ m/z=1517.72, [M+11H]¹¹⁺ m/z=1379.84, [M+12H]¹²⁺+m/z=1264.94, [M+13H]¹³⁺ m/z=1167.71, [M+14H]¹⁴⁺ m/z=1084.37, [M+15H]¹⁵ m/z=1012.15, [M+16H]¹⁶⁺ m/z=948.95; observed: [M+8H]⁸⁺ m/z=1896.57, [M+9H]⁹⁺ m/z=1686.16, [M+10H]¹⁰⁺ m/z=1517.59, [M+11H]¹¹⁺ m/z=1379.51, [M+12H]¹²⁺ m/z=1264.79, [M+13H]¹³⁺ m/z=1167.43, [M+14H]¹⁴⁺ m/z=1084.07, [M+15H]¹⁵⁺ m/z=1011.83, [M+16H]¹⁶⁺ m/z=948.59.

### GM-CSF Analogue Primary Construct 22

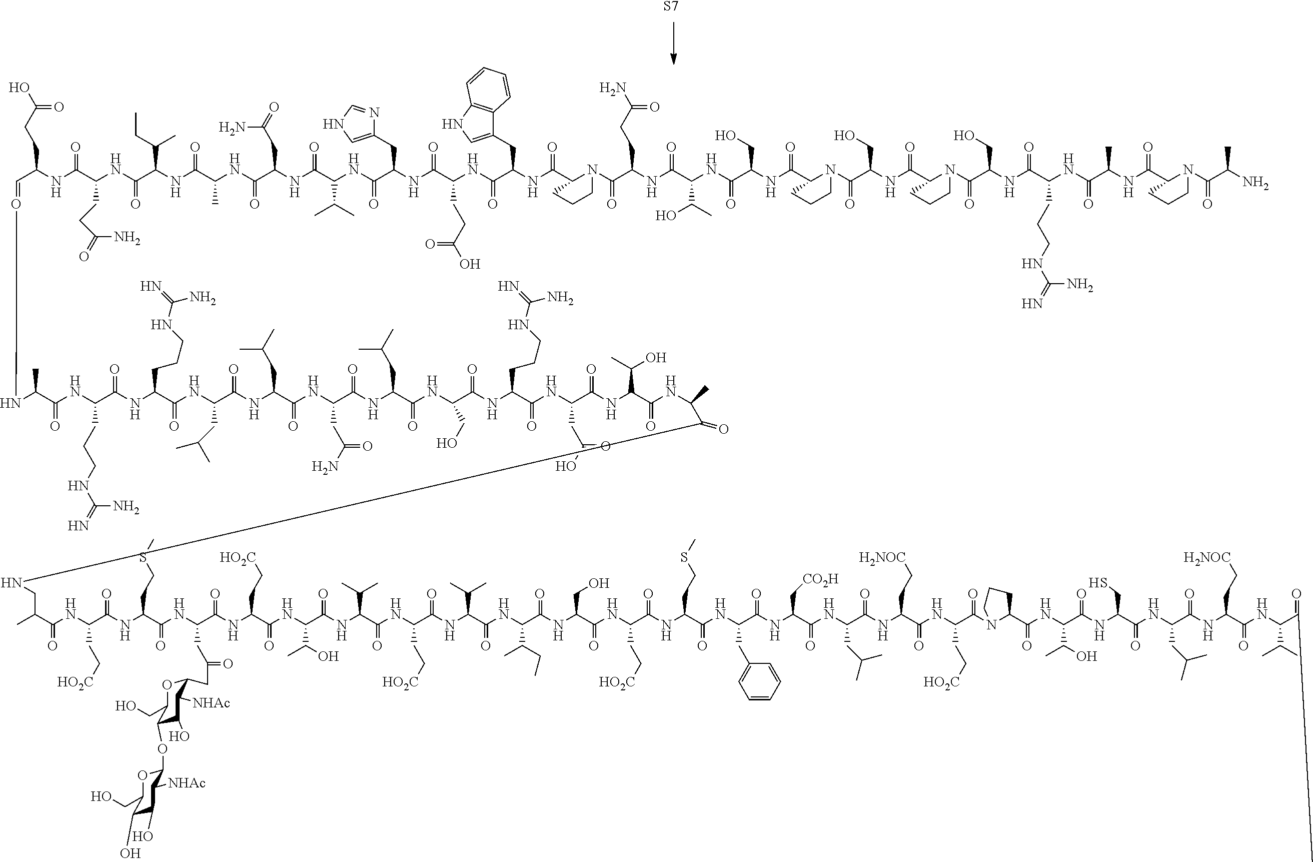

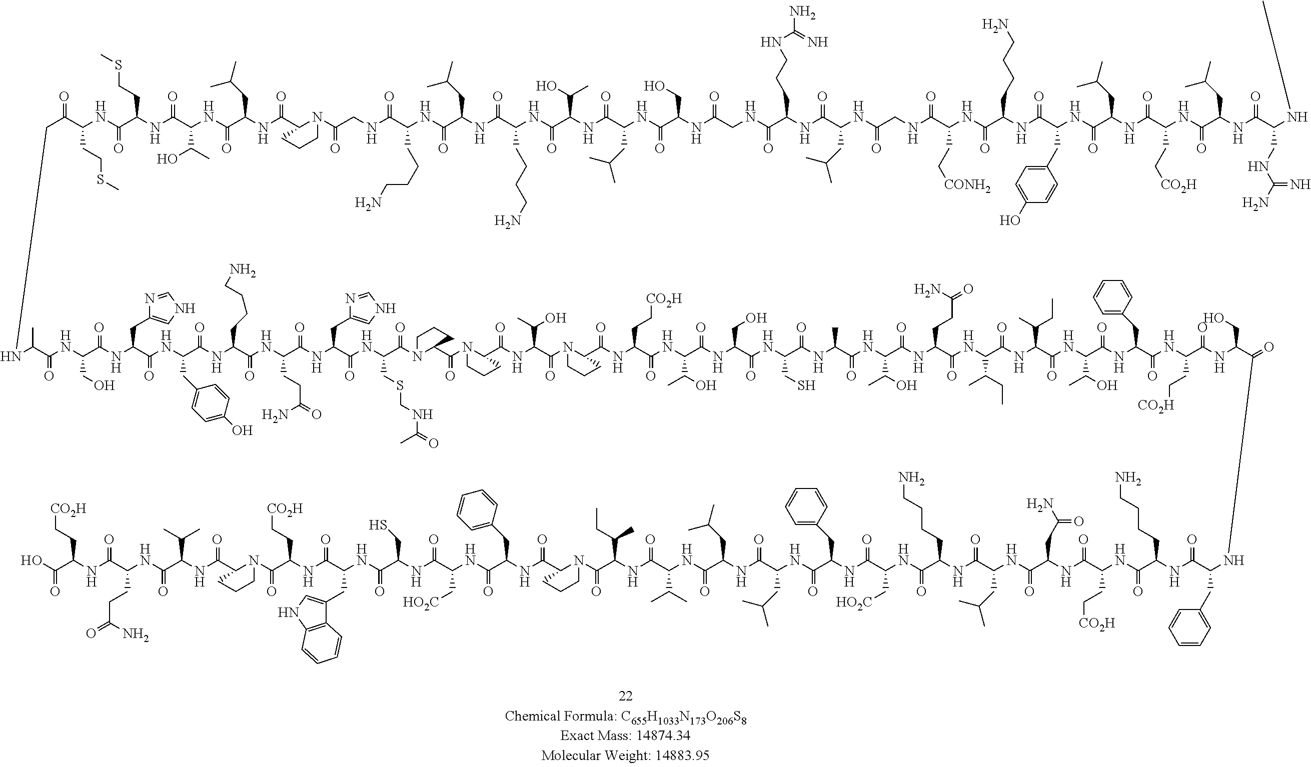

According to General Procedure F, to glycopeptide S7 (1.50 mg, 1.0 equiv) in 0.2 ml of degassed solvent HOAc: H₂O (3:1) was added AgOAc (3.0 mg, 200 equiv) in one portion. The reaction mixture was stirred at rt and monitored by LC-MS. Upon completion, the reaction was quenched by the addition of 0.2 mL of DTT in H₂O/AcOH (1:1, 1 mM), the result cloudy mixture was stirred for 20 min. Mixture was centrifuged and the supernatant was carefully taken out and lyophilized to get white solid.

The product was purified directly by RP-HPLC (linear gradient 40-60% solvent B over 30 min, Microsorb 300-5 C4 column, 16 mL/min, 230 nm). Product eluted at 14-17 min. The fractions were collected, and concentrated via lyophilization to afford 0.80 mg ligated peptide 22 (54%) as a white solid. ESI-MS analysis of glycopeptide 22: Calcd for C₆₅₅H₁₀₃₃N₁₇₃O₂₀₆S₈: 14883.95 Da(average isotopes), [M+10H]¹⁰⁺ m/z=1489.39, [M+11H]¹¹⁺ m/z=1354.08, [M+12H]¹²⁺ m/z=1241.32, [M+13H]¹³⁺ m/z=1145.91, [M+14H]¹⁴⁺ m/z=1064.13, [M+15H]¹⁵⁺ m/z=993.26, [M+16H]¹⁶⁺ m/z=931.24, [M+17H]¹⁷⁺ m/z=876.52; observed: [M+10H]¹⁰⁺ m/z=1489.10, [M+11H]¹¹⁺ m/z=1353.90, [M+12H]¹²⁺ m/z=1240.90, [M+13H]¹³⁺ m/z=1145.70, [M+14H]¹⁴⁺ m/z=1065.10, [M+15H]¹⁵⁺ m/z=993.20, [M+16H]¹⁶⁺ m/z=930.90, [M+17H]¹⁷⁺ m/z=876.20.

### GM-CSF Glycopeptide Full Sequence 18

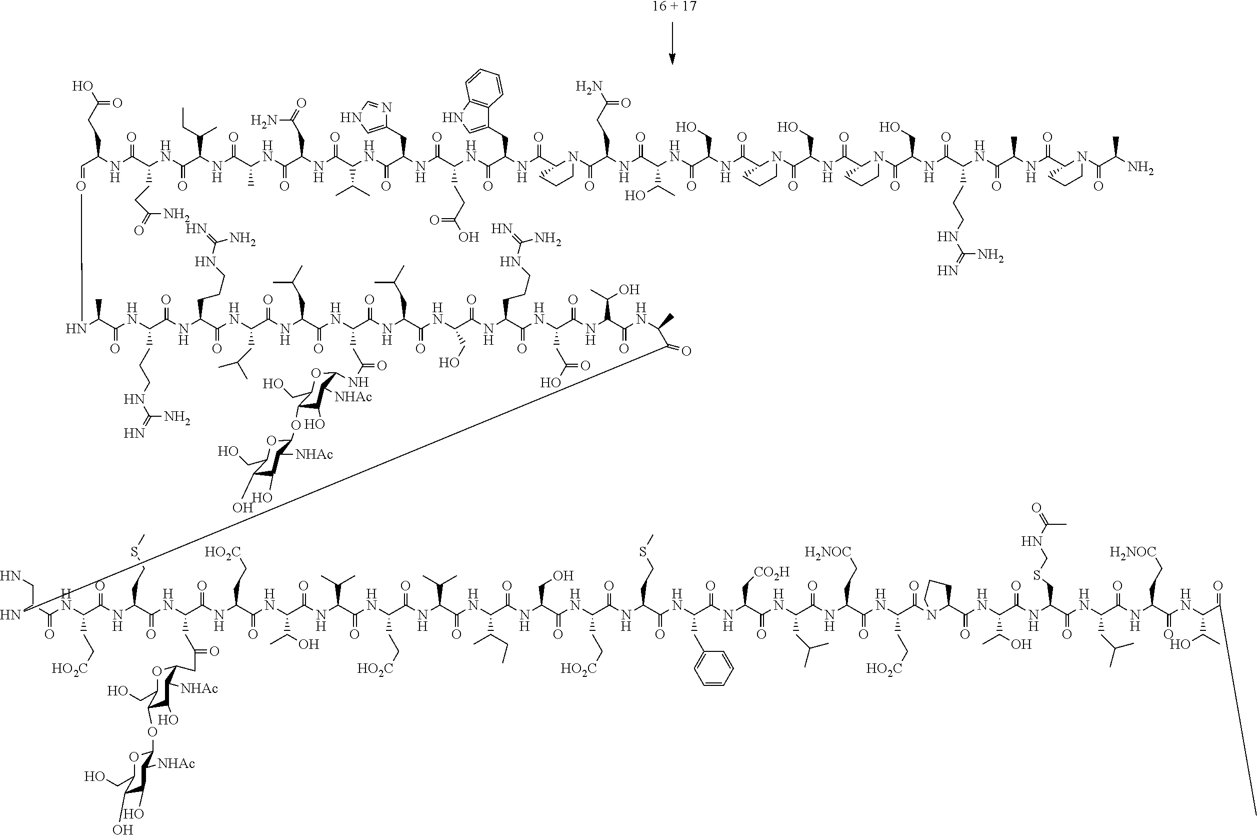

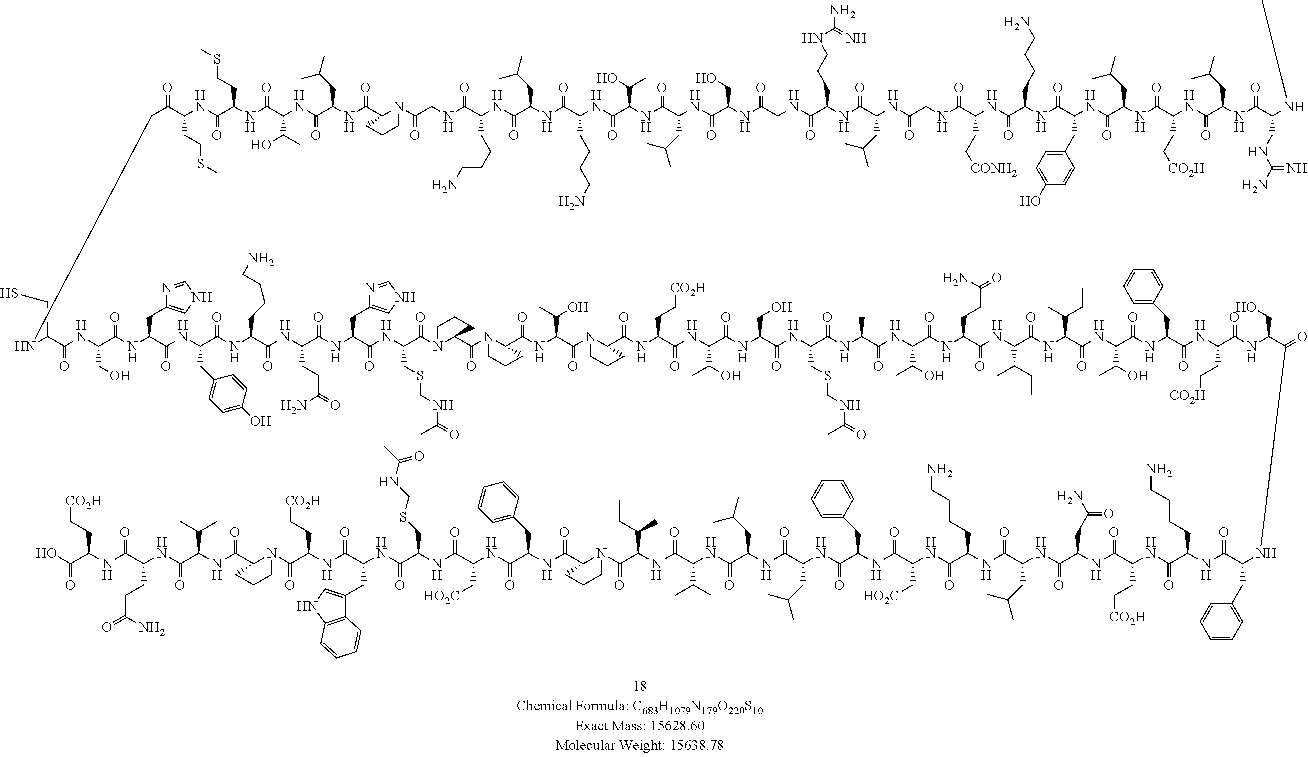

According to General Procedure D, glycopeptides 16 (4.00 mg, 1.00 equiv) and 17 (2.13 mg, 1.50 equiv) were dissolved in 200 μL of NCL buffer under an argon atmosphere. The resulting mixture was stirred at room temperature and the reaction was monitored by LC-MS. After 15 h, the reaction was added 0.2 ml of 0.5 M Bond-Breaker® TCEP solution and diluted with 3 mL of 5% AcOH, and then purified directly by RP-HPLC (linear gradient 35-60% solvent B over 30 min, Microsorb 300-5 C4 column, 16 mL/min, 230 nm). Product eluted at 17-22 min. The fractions were collected, and concentrated via lyophilization to afford 2.26 mg ligated peptide 18 (42%) as a white solid. ESI-MS analysis of glycopeptide 18: Calcd for C₆₈₃H₁₀₇₉N₁₇₉O₂₂₀S₁₀: 15638.78 Da (average isotopes), [M+8H]⁸⁺ m/z=1955.84, [M+9H]⁹⁺ m/z=1738.64, [M+10H]¹⁰⁺ m/z=1564.88, [M+11H]¹¹⁺ m/z=1422.71, [M+12H]¹²⁺+m/z=1304.23, [M+13H]¹³⁺ m/z=1203.98, [M+14H]¹⁴⁺ m/z=1118.05, [M+15H]¹⁵⁺ m/z=1043.58, [M+16H]¹⁶⁺ m/z=978.42, [M+17H]¹⁷⁺ m/z=920.93; observed: [M+8H]⁸⁺ m/z=1955.70, [M+9H]⁹⁺ m/z=1738.60, [M+10H]¹⁰⁺ m/z=1564.80, [M+11H]¹¹⁺ m/z=1422.60, [M+12H]¹²⁺ m/z=1304.20, [M+13H]¹³⁺ m/z=1203.80, [M+14H]¹⁴⁺ m/z=1117.90, [M+15H]¹⁵⁺ m/z=1043.50, [M+16H]¹⁶⁺ m/z=978.30, [M+17H]¹⁷⁺ m/z=920.70.

### GM-CSF Glycopeptide ACM Primary Construct 19

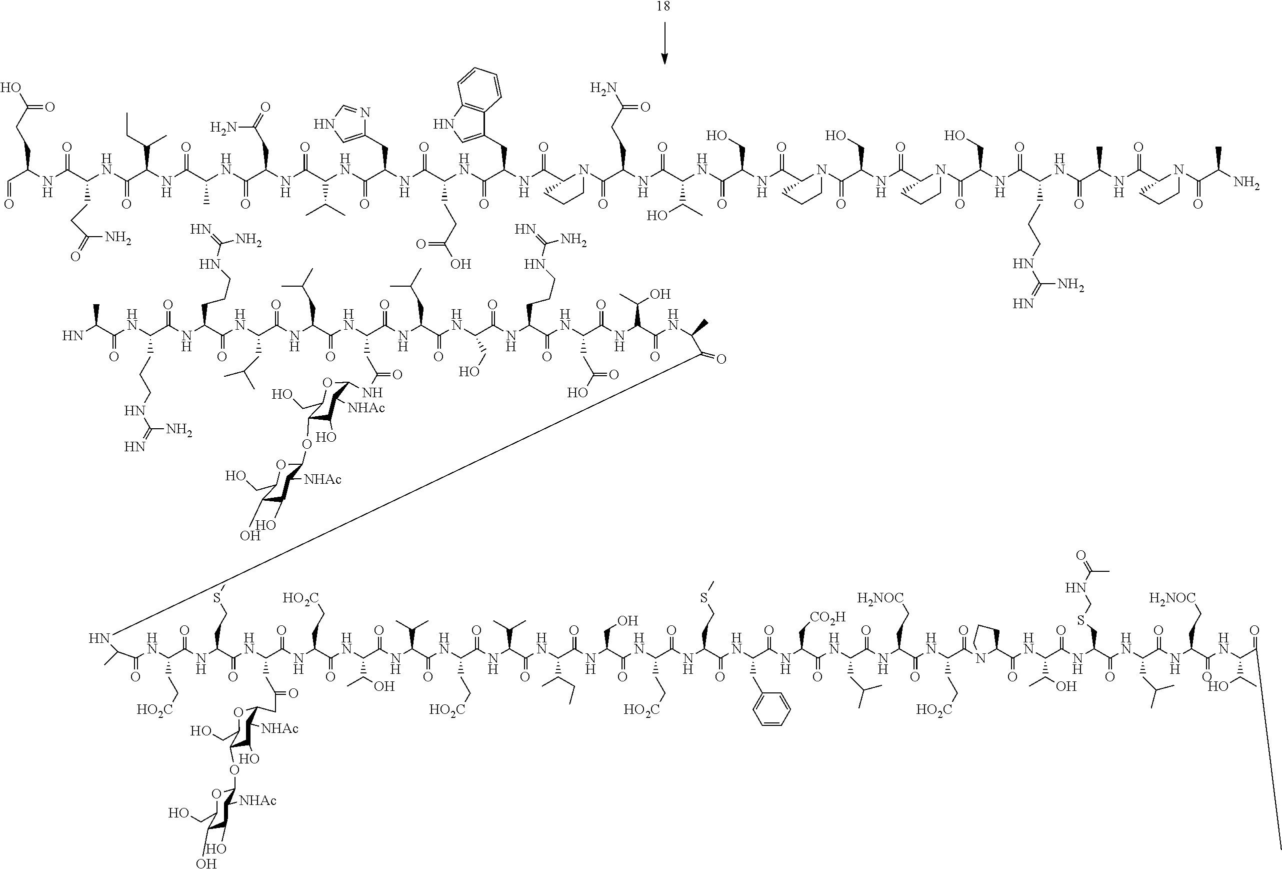

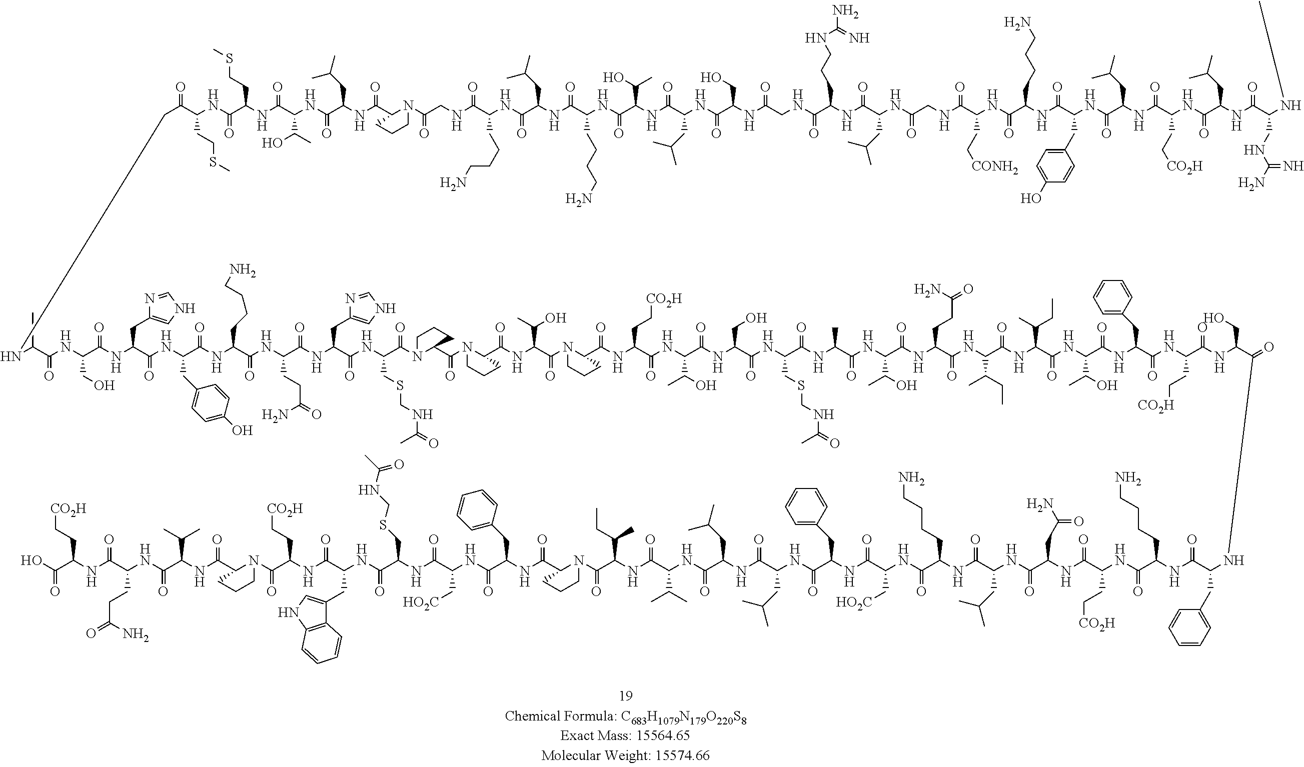

According to General Procedure E, to glycopeptide 18 (1.80 mg,) in 0.3 ml of degassed buffer (6 M Gnd.HCl, 200 mM Na₂HPO₄) was added 0.2 ml of 0.5 M Bond-Breaker® TCEP solution (Pierce), 400 μL of 2-methyl-2-propanethiol and 750 μL of radical initiator VA-044 (0.1 M in H₂O). The reaction was stirred at 37° C. under an argon atmosphere for 5 h. The resulting mixture was diluted with CH₃CN/H₂O/AcOH (47.5:47.5:5), and then purified directly by RP-HPLC (linear gradient 40-60% solvent B over 30 min, Microsorb 300-5 C4 column, 16 mL/min, 230 nm). Product eluted at 13-17 min. The fractions were collected, and concentrated via lyophilization to afford 1.22 mg peptide 19 (68%) as a white solid. ESI-MS analysis of glycopeptide 19: Calcd for C₆₈₃H₁₀₇₉N₁₇₉O₂₂₀S₈: 15574.66 Da(average isotopes), [M+8H]⁸⁺ m/z=1947.83, [M+9H]⁹⁺ m/z=1731.52, [M+10H]¹⁰⁺ m/z=1558.46, [M+11H]¹¹⁺ m/z=1416.88, [M+12H]¹²⁺ m/z=1298.88, [M+13H]¹³⁺ m/z=1199.05, [M+14H]¹⁴⁺ m/z=1113.47, [M+15H]¹⁵⁺ m/z=1039.31, [M+16H]¹⁶⁺ m/z=974.41, [M+17H]¹⁷⁺ m/z=917.15; observed: [M+8H]⁸⁺ m/z=1947.60, [M+9H]⁹⁺ m/z=1731.40, [M+10H]¹⁰⁺ m/z=1558.30, [M+11H]¹¹⁺ m/z=1416.70, [M+12H]¹²⁺ m/z=1298.80, [M+13H]¹³⁺ m/z=1198.90, [M+14H]¹⁴⁺ m/z=1113.30, [M+15H]¹⁵⁺ m/z=1039.20, [M+16H]¹⁶⁺ m/z=974.30, [M+17H]¹⁷⁺ m/z=917.10.

### GM-CSF Glycopeptide Primary Construct 20

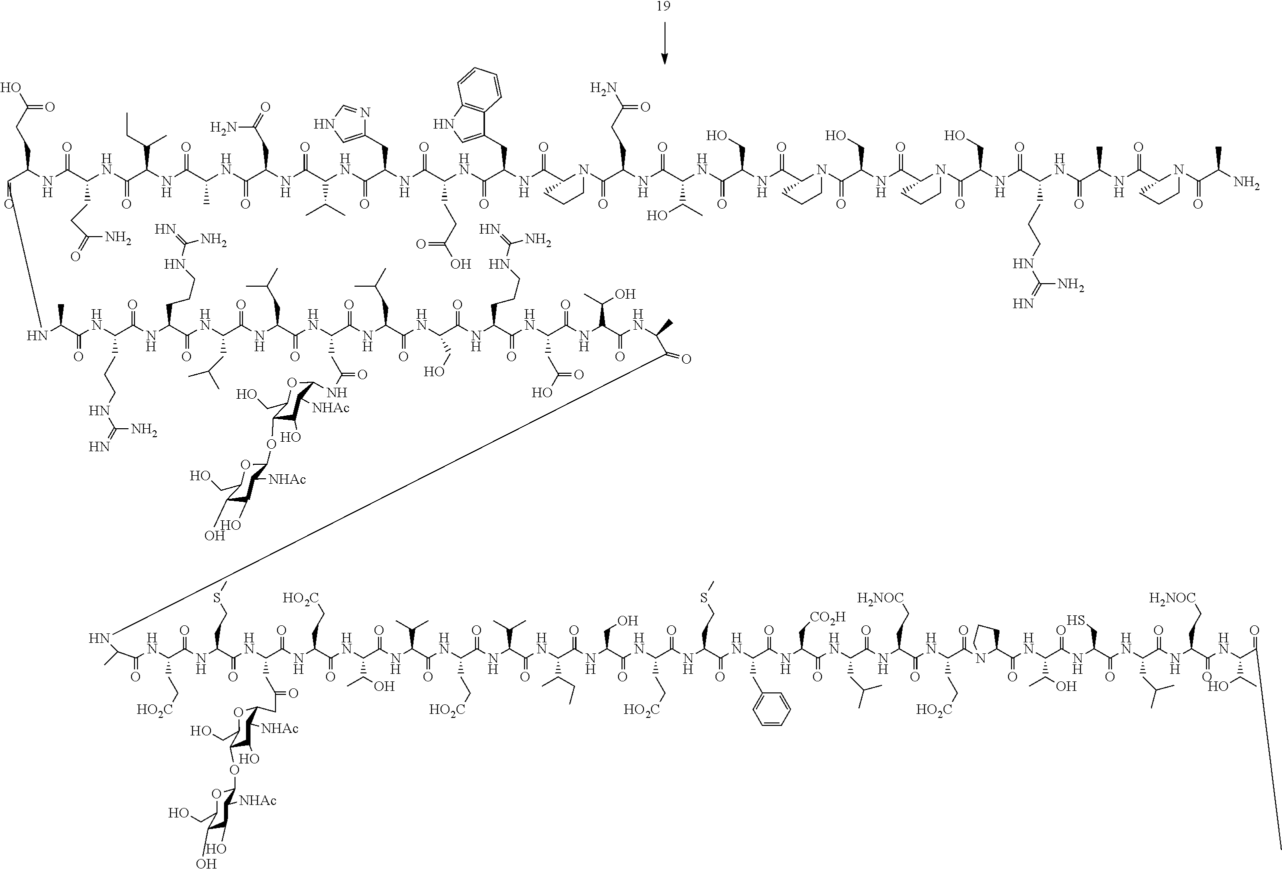

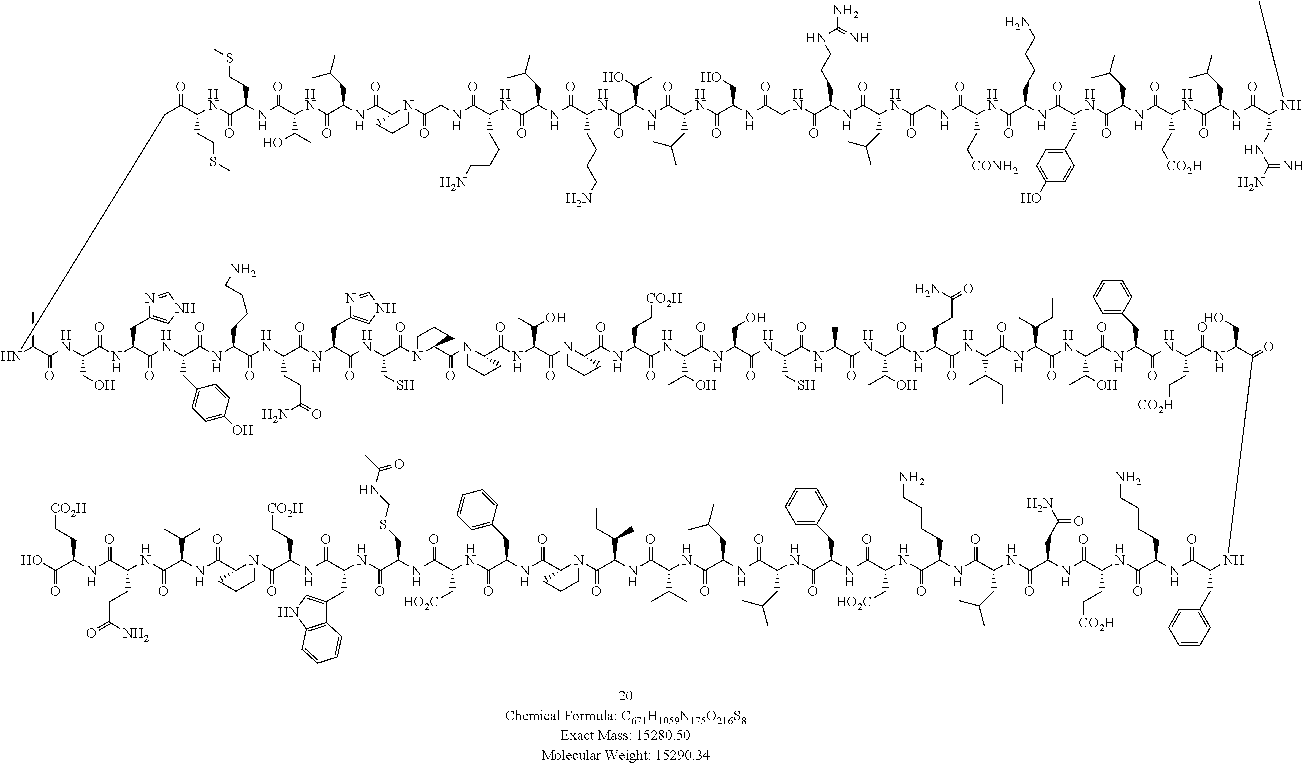

According to General Procedure F, to a solution of glycopeptide 19 (1.00 mg, 1 equiv) in 200 μL of degassed AcOH/H₂O (3:1), was added AgOAc (2.1 mg, 200 equiv) in one portion. The resulting mixture was stirred at rt under an argon atmosphere for 90 mins. The reaction was quenched by the addition of 0.2 mL of DTT in H₂O/AcOH (1:1, 1 M), the result cloudy mixture was stirred for 20 min), the resulting mixture was further stirred for 30 min, followed by centrifugation. The supernatant was carefully taken out and solid were washed with 0.2 mL of DTT in H₂O/AcOH (1:1, 1 mM) two more times. All supernatant was collected and lyophilized to give white solid. The product purified directly by RP-HPLC (linear gradient 40-55% solvent B over 30 min, Microsorb 300-5 C4 column, 16 mL/min, 230 nm). Product eluted at 14-18 min. The fractions were collected, and concentrated via lyophilization to afford 0.59 mg ligated peptide 20 (60%) as a white solid. ESI-MS analysis of glycopeptide 20: Calcd for C₆₇₁H₁₀₅₉N₁₇₅O₂₁₆S₈: 15290.34 Da (average isotopes), [M+9H]⁹⁺ m/z=1699.92, [M+10H]¹⁰⁺ m/z=1530.00, [M+11H]¹¹⁺ m/z=1391.03, [M+12H]¹²⁺ m/z=1275.19, [M+13H]¹³⁺ m/z=1177.18, [M+14H]¹⁴⁺ m/z=1093.16, [M+15H]¹⁵⁺ m/z=1020.35, [M+16H]¹⁶⁺ m/z=956.64, [M+17H]¹⁷⁺ m/z=900.43; observed: [M+9H]⁹⁺ m/z=1699.90, [M+10H]¹⁰⁺ m/z=1530.00, [M+11H]¹¹⁺ m/z=1391.10, [M+12H]¹²⁺ m/z=1275.20, [M+13H]¹³⁺ m/z=1178.20, [M+14H]¹⁴⁺ m/z=1093.30, [M+15H]¹⁵⁺ m/z=1021.00, [M+16H]¹⁶⁺ m/z=956.70, [M+17H]¹⁷⁺ m/z=901.20.

### Example 4

### Folding of GM-CSF

GM-CSF primary construct (0.3 mg) was dissolved in 50 mM Tris, pH 7.5, 2 M GuHCl (0.2 mL), and the resulting solution was injected in a dialysis cassette (0.1-0.5 mL, 7,000 MWCO, Pierce). The cassette was placed in 400 mL dialysis buffer #1 (50 mM Tris, pH 8, 1 M GuHCl, 0.4 M Arginine (Sigma, A5006), 3 mM Reduced Glutathione, 0.9 mM Oxidized Glutathione) and stirred for 24 h at 4° C. The following day the dialysis buffer was diluted 50% with water and dialysis continued for another 24 h. On day 3, the cassette was dialyzed for 24 h at 4° C. against 200 mL of dialysis buffer #3 (50 mM Tris, pH 8, 250 mM NaCl, 0.1 M Arginine, 3 mM Reduced Glutathione, 0.9 mM Oxidized Glutathione). The dialyzed protein was direct purified by RP-HPLC (linear gradient 40-55% solvent B over 30 min, Microsorb 300-5 C4 column, 16 mL/min, 230 nm). Product eluted at 12-16 min. The fractions were collected, and concentrated via lyophilization to afford 80 μg of folded GM-CSF protein 10 (27%) as a white solid. ESI-MS analysis of glycopeptide 10: Calcd for C₆₇₁H₁₀₅₉N₁₇₅O₂₁₆S₈: 15290.34 Da (average isotopes), [M+9H]⁹⁺ m/z=1699.92, [M+10H]¹⁰⁺ m/z=1530.00, [M+11H]¹¹⁺ m/z=1391.03, [M+12H]¹²⁺ m/z=1275.19, [M+13H]¹³⁺ m/z=1177.18, [M+14H]¹⁴⁺ m/z=1093.16, [M+15H]¹⁵⁺ m/z=1020.35, [M+16H]¹⁶⁺ m/z=956.64, [M+17H]¹⁷⁺ m/z=900.43; observed: [M+9H]⁹⁺ m/z=1699.90, [M+10H]¹⁰⁺ m/z=1530.00, [M+11H]¹¹⁺ m/z=1391.10, [M+12H]¹² m/z=1275.20, [M+13H]¹³⁺ m/z=1178.20, [M+14H]¹⁴⁺ m/z=1093.30, [M+15H]¹⁵⁺ m/z=1021.00, [M+16H]¹⁶⁺ m/z=956.70, [M+17H]¹⁷⁺ m/z=901.20.

Other GM-CSF constructs, such as GM-CSF 21, were folded using the same procedure.

### CD Spectra:

CD spectra were obtained on an Aviv® 410 circular dichroism spectropolarimeter. Protein concentration (˜1.0 μM) were determined based on the extinction coefficient, calculated according to the number of Trp residue (Edelhoch H. Spectroscopic determination of tryptophan and tyrosine in proteins. Biochemistry 1967, 6, 1948). Sample was dissolved in 10 mM phosphate buffer solution (pH 7.2) and the spectra were collected using a 1 mm pathlength cuvette. See FIG. S23.

### Example 5

### Effect of Recombinant GM-CSF and Synthetic GM-CSF on Proliferation of TF-1 Cells

2,000 TF-1 cells/50 μl of a IMDM medium containing 20% SR with or without various dose of recombinant GM-CSF (Leukine, Sanofi-Aventis) or synthetic GM-CSF in 384-wells plate in triplicates. After 4 days incubation, the cultures were pulsed with Alamar Blue (Life Technologies. Grand Island, N.Y.) overnight and measured fluorescence intensity by Synergy H1 plate reader (BioTek Inc, Winooski, Vt.). The results are expressed as Mean of Relative Fluorescence Intensity±S.D., n=3. Relative Fluorescence Intensity=Fluorescence Intensity of TF-1 cultures with various dose of GM-CSF/Fluorescence Intensity of TF-1 cultures with 125 pg/ml of Leukine GM-CSF. See FIG. S20.

### Example 6

### Effect of Recombinant GM-CSF and Synthetic GM-CSF on Colony Formation in Cord Blood CD34+Cells

1,000 CB CD34+ cells were cultured in 1 ml IMDM containing 1.2% methylcellulose, 30% Knockout Serum Replacement (Life Technologies, Grand Island, N.Y.), 0.1 mM 2-mercaptoethanol, 2 mM glutamine, 50 units/ml penicillin, 50 μg/ml streptomycin, 20 ng/ml KL and with or without various doses of recombinant GM-CSF (Peptro GM) and synthetic GM-CSF analogue in 5% CO₂ humidified incubator at 37° C. in triplicates. After 14 days, CFC were scored under microscope. No colony was formed in KL alone group. See FIG. S21.

### Example 7

Colony-forming Cells (CFC) bioassay is performed by culturing 1,000 purified human umbilical cord blood CD34⁺ cells/ml of IMDM containing 1.2% methylcellulose, 80 uM 2-mercaptoethanol, 2 mM L-glutamine, 50 units/ml penicillin, 50 ug/ml streptomycin, 0.125 mM hemin (Sigma), and 20% serum replacement (Life Technology, Grand Island, N.Y.) in the presence or absence of various dose of human recombinant GM-CSF (Sanofi-Aventis U.S. LLC, Bridgewater, N.J.) or synthetic GM-CSFs in triplicates. After 14 days, the colonies containing more than 50 cells/colony CFC are scored as CFC under a microscope and data are expressed as Mean±S.D., n=3.

In FIG. S22, the images of CFC were acquired in a Nikon Eclipse Ti microscope equipped with a Nikon Digital Sight camera. The picture on the left is the image of cell growth when treating cell with commercially available GM-CSF, the picture on the right is the cell growth image when treating cell with synthetic GM-CSF 21.

1. A composition of homogeneously glycosylated GM-CSF or a homogeneously glycosylated fragment thereof, wherein each molecule of GM-CSF or fragment thereof has the same glycosylation pattern, and for a given glycosylation site each molecule of GM-CSF or fragment thereof has the same glycan.

2. The composition of claim 1, comprising a polypeptide whose amino acid sequence includes a sequence that:
a) is identical to that of:
(SEQ ID NO: 1)
Ala-Pro-Ala-Arg-Ser-Pro-Ser-Pro-Ser-Thr-Gln-Pro-
Trp-Glu-His-Val-Asn-Ala-Ile-Gln-Glu-Ala-Arg-Arg-
Leu-Leu-Asn-Leu-Ser-Arg-Asp-Thr-Ala-Ala-Glu-Met-
Asn-Glu-Thr-Val-Glu-Val-Ile-Ser-Glu-Met-Phe-Asp-
Leu-Gln-Glu-Pro-Thr-Cys-Leu-Gln-Thr-Arg-Leu-Glu-
Leu-Tyr-Lys-Gln-Gly-Leu-Arg-Gly-Ser-Leu-Thr-Lys-
Leu-Lys-Gly-Pro-Leu-Thr-Met-Met-Ala-Ser-His-Tyr-
Lys-Gln-His-Cys-Pro-Pro-Thr-Pro-Glu-Thr-Ser-Cys-
Ala-Thr-Gln-Ile-Ile-Thr-Phe-Glu-Ser-Phe-Lys-Glu-
Asn-Leu-Lys-Asp-Phe-Leu-Leu-Val-Ile-Pro-Phe-Asp-
Cys-Trp-Glu-Pro-Val-Gln-Glu,
or
b) contains 1, 2, 3, 4, 5, 6, 7, 8, 9, or 10 amino acid deletions, substitutions, additions or combinations thereof relative to such SEQ ID NO: 1, or
c) is a fragment of a) or b), wherein the fragment has an amino acid sequence corresponding to amino acid residues 1-33, 34-53, 34-80, 54-95, 81-127, or 96-127 of SEQ ID NO: 1, or contains 1, 2, 3, 4, 5, 6, 7, 8, 9, or 10 amino acid deletions, substitutions, additions or combinations thereof relative to such fragment;
the polypeptide having at least one amino acid residue site glycosylated;
wherein each glycosylated polypeptide in the composition has the same glycosylation pattern in that:
it is glycosylated on at least one amino acid residue site;
it is glycosylated at the same at least one site;
it is glycosylated at a site selected from the group consisting of Asn²⁷, Asn³⁷, Ser⁵, Ser⁷, Ser⁹, and Thr¹⁰, in SEQ ID NO:1, and combinations thereof; and
for a given glycosylation site, it has the same glycan.

3. The composition of claim 1 or 2, wherein the polypeptide's amino acid sequence is identical to that of SEQ ID NO: 1.

4. The composition of claim 1 or 2, wherein the polypeptide's amino acid sequence is SEQ ID NO: 1 having 1, 2, 3, 4, 5, 6, 7, 8, 9, or 10 amino acid deletions, substitutions, additions or combinations thereof.

5. The composition of claim 2, wherein the polypeptide's amino acid sequence includes a sequence that contains one or more modifications relative to that of SEQ ID NO: 1, wherein at least one such modification prevents or decreases the polypeptide's susceptibility to truncation relative to that of a polypeptide whose sequence is identical to SEQ ID NO: 1.

6. The composition of claim 5, wherein the modification is the addition of or substitution with one, two, three, four, five, six, seven, or more unnatural amino acids.

7. The composition of claim 5 or 6, wherein the modification is glycosylation of one or more amino acid residues.

8. The composition of claim 5, 6, or 7, wherein the modification prevents or decreases the polypeptide's susceptibility to truncation at the N-terminus relative to that of a polypeptide whose sequence is identical to SEQ ID NO: 1.

9. The composition of claim 8, wherein the modification prevents or decreases the polypeptide's susceptibility to truncation by dipeptidyl peptidase 4 relative to that of a polypeptide whose sequence is identical to SEQ ID NO: 1.

10. The composition of claim 1 or 2, wherein the fragment has an amino acid sequence corresponding to amino acid residues 1-33, 34-53, 34-80, 54-95, 81-127, or 96-127 of SEQ ID NO: 1, or contains 1, 2, 3, 4, 5, 6, 7, 8, 9, or 10 amino acid deletions, substitutions, additions or combinations thereof relative to such fragment.

11. The composition of claim 1 or 2, wherein the structure of the fragment is selected from:

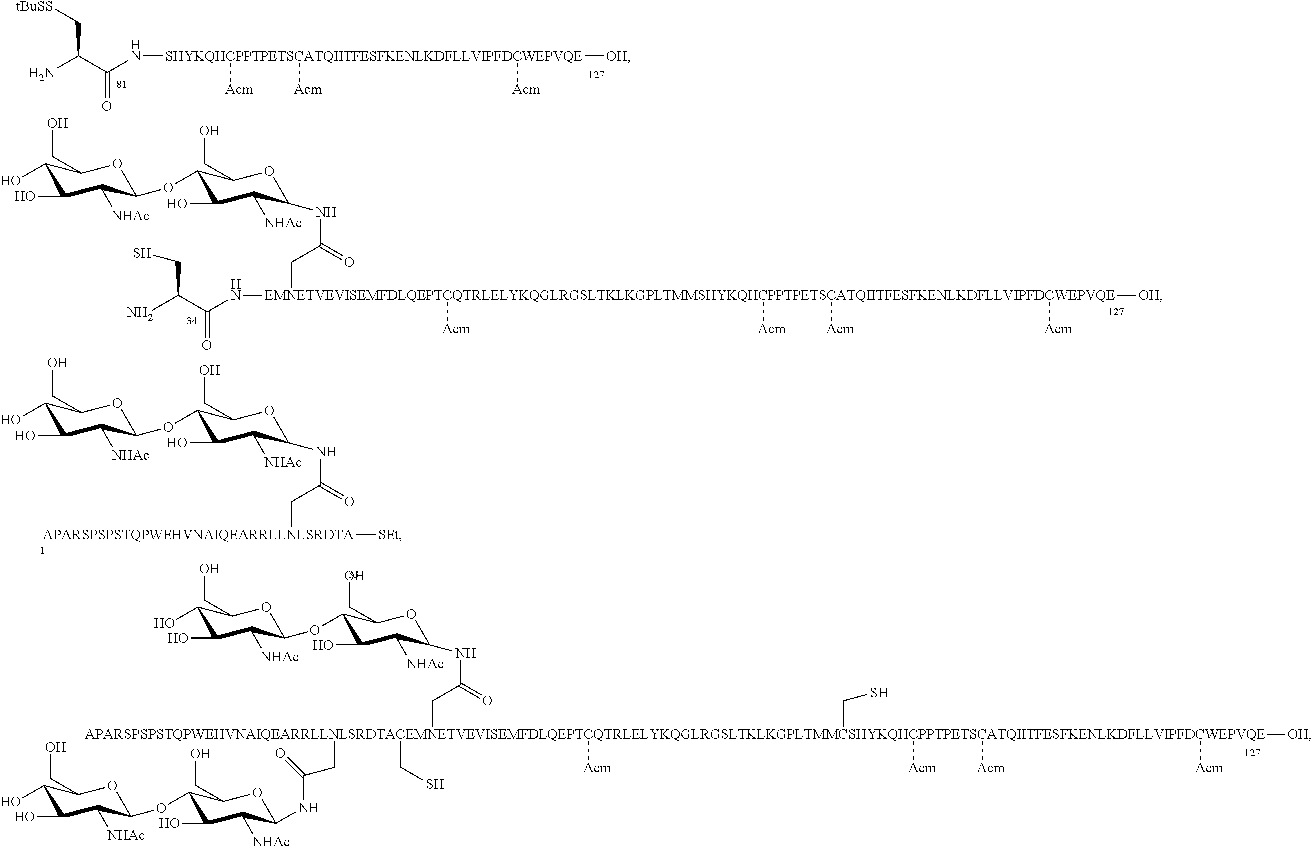

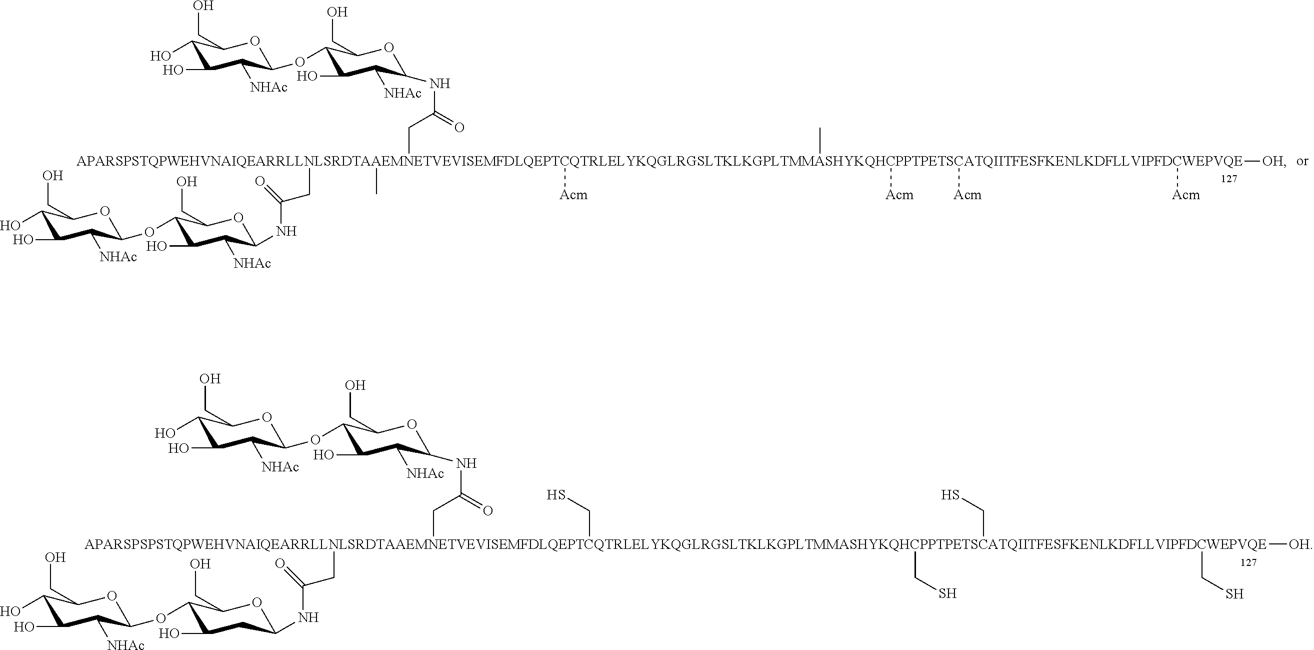

12. A polypeptide whose amino acid sequence includes a sequence that contains one or more modifications relative to that of SEQ ID NO: 1, wherein at least one such modification prevents or decreases the polypeptide's susceptibility to truncation relative to that of a polypeptide whose sequence is identical to SEQ ID NO: 1.

13. The polypeptide of claim 12, wherein the modification is the addition of or substitution with one, two, three, four, five, six, seven, or more unnatural amino acids.

14. The polypeptide of claim 12 or 13, wherein the modification is glycosylation of one or more amino acid residues.

15. The polypeptide of claim 12, 13, or 14, wherein the modification prevents or decreases the polypeptide's susceptibility to truncation at the N-terminus relative to that of a polypeptide whose sequence is identical to SEQ ID NO: 1.

16. The polypeptide of claim 15, wherein the modification prevents or decreases the polypeptide's susceptibility to truncation by dipeptidyl peptidase 4 relative to that of a polypeptide whose sequence is identical to SEQ ID NO: 1.

17. The polypeptide of any one of claims 12-15, further comprising 1, 2, 3, 4, 5, 6, 7, 8, 9, or 10 amino acid deletions, substitutions, additions or combinations thereof relative to such SEQ ID NO: 1.

18. The composition or polypeptide of any preceding claim, wherein the polypeptide stimulates white blood cell production.

19. A prodrug of the homogeneously glycosylated GM-CSF or the homogeneously glycosylated fragment thereof of any one of claims 1-18, wherein the GM-CSF polypeptide's C- or N-terminus is modified such that, upon suitable in vivo bioactivation, the prodrug is converted to an active form of GM-CSF.

20. The prodrug of claim 19, wherein the GM-CSF polypeptide's C- or N-terminus is extended by a peptide or modified peptide sequence that is cleaved upon suitable in vivo bioactivation to yield an active form of GM-CSF.

21. A method of stimulating white blood cell production comprising administering to a patient in need thereof a composition of claims 1-11, a polypeptide of claims 12-17, a composition of claim 18, or a prodrug of claims 19-20.

22. The method of claim 21, wherein the patient is infected with HIV.

23. The method of claim 21, wherein the patient is being treated or has been treated with chemotherapy.

24. The method of claim 21, wherein the patient has undergone autologous bone marrow transplant.

25. The method of claim 21, wherein the patient is immune compromised.

26. A method of enhancing the immune response to a cancer vaccine comprising administering to a patient in need thereof a composition of claims 1-11, a polypeptide of claims 12-17, a composition of claim 18, or a prodrug of claims 19-20.

27. The method of claim 26, wherein the patient has been diagnosed with cancer.

28. The method of claim 26 or 27, comprising co-administration with a cancer vaccine.

29. The method of claims 26-28, wherein the cancer vaccine comprises one or more carbohydrates.

30. A method of preparing GM-CSF, the method comprising the step of:
ligating to one another a set of fragments of a polypeptide whose amino acid sequence includes a sequence that:
a) is identical to that of:
(SEQ ID NO: 1)
Ala-Pro-Ala-Arg-Ser-Pro-Ser-Pro-Ser-Thr-Gln-Pro-
Trp-Glu-His-Val-Asn-Ala-Ile-Gln-Glu-Ala-Arg-Arg-
Leu-Leu-Asn-Leu-Ser-Arg-Asp-Thr-Ala-Ala-Glu-Met-
Asn-Glu-Thr-Val-Glu-Val-Ile-Ser-Glu-Met-Phe-Asp-
Leu-Gln-Glu-Pro-Thr-Cys-Leu-Gln-Thr-Arg-Leu-Glu-
Leu-Tyr-Lys-Gln-Gly-Leu-Arg-Gly-Ser-Leu-Thr-Lys-
Leu-Lys-Gly-Pro-Leu-Thr-Met-Met-Ala-Ser-His-Tyr-
Lys-Gln-His-Cys-Pro-Pro-Thr-Pro-Glu-Thr-Ser-Cys-
Ala-Thr-Gln-Ile-Ile-Thr-Phe-Glu-Ser-Phe-Lys-Glu-
Asn-Leu-Lys-Asp-Phe-Leu-Leu-Val-Ile-Pro-Phe-Asp-
Cys-Trp-Glu-Pro-Val-Gln-Glu
which set of fragments includes fragments whose amino acid sequence corresponds to amino acid residues 1-33, 34-53, 34-80, 54-95, 81-127, or 96-127 of SEQ ID NO: 1, so that a homogenously glycosylated GM-CSF polypeptide is generated.

31. The method of claim 30, wherein each molecule of GM-CSF or fragment thereof has the same glycosylation pattern, and for a given glycosylation site each molecule of GM-CSF or fragment thereof has the same glycan.
